(19) United States
(12) Patent Application Publication
(10) Pub. No.: US 2026/0108512 A1
(43) Pub. Date: Apr. 23, 2026

(54) COMPOUNDS, COMPOSITIONS AND METHODS OF TREATING DISORDERS

(71) Applicant: HOTSPOT THERAPEUTICS, INC., Boston, MA (US)

(72) Inventors: Yingzhi BI, Plainsboro, NJ (US); Kenneth G. CARSON, Princeton, NJ (US); Geraldine Cirillo HARRIMAN, Charlestown, RI (US)

(21) Appl. No.: 19/172,091

(22) Filed: Apr. 7, 2025

Related U.S. Application Data
(63) Continuation of application No. PCT/US2023/076236, filed on Oct. 6, 2023.
(60) Provisional application No. 63/414,257, filed on Oct. 7, 2022.

(51) Int. Cl. A61K 31/501; A61K 31/437; A61P 35/00; C07D 471/04

(52) U.S. Cl. CPC A61K 31/501; A61K 31/437; A61P 35/00; C07D 471/04

(57) ABSTRACT

The present disclose includes, among other things, compounds that treat or lessen the severity of cancer, pharmaceutical compositions and methods of making and using the same.

### CROSS REFERENCE TO RELATED APPLICATION

This application claims priority to U.S. Provisional Application No. 63/414,257 filed Oct. 7, 2022, the contents of which are incorporated herein by reference.

### BACKGROUND

Cbl-b is a E3 ubiquitin-protein ligase that functions as a negative regulator of T-cell activation. Modulation of Cbl-b has been shown to be a therapeutic target for a diseases and disorders. There remains a need for compounds that inhibit Cbl-b.

### SUMMARY

In some embodiments, the present disclosure includes a compound of formula (I):

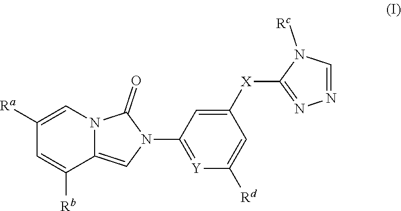

or a pharmaceutically acceptable salt thereof.

Additionally, the present disclosure includes, among other things, pharmaceutical compositions, methods of using and methods of making a compound of formula (I).

### DETAILED DESCRIPTION

In some embodiments, the present disclosure includes a compound of formula (I):

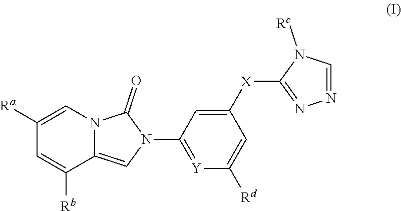

or pharmaceutically acceptable salts thereof,
   wherein
X is an optionally substituted C₁-C₃ alkylene chain, wherein one or more methylene units is optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic;
Y is selected from N or C;
R^{a} is selected from the group consisting of —CH₂-A, —CH₂N(H)-A, and —CH₂NH₂;
R^{b} is halogen or optionally substituted C₁-C₃ alkyl;
each R^{c} is optionally substituted C₁-C₃ alkyl;
R^{d} is selected from —NR¹R² or hydrogen;
A is optionally substituted C₃-C₇ carbocylyl or optionally substituted 3-6 membered heterocyclyl containing 1-4 heteroatoms each selected from the group consisting of N, O, and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S,
wherein two instances of R^{a1} are optionally taken together to form an optionally substituted C₃-C₇ carbocylyl or optionally substituted 3-6 membered heterocyclyl containing 1-4 heteroatoms each selected from the group consisting of N, O, and S;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S; and
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes a compound of formula (I-a):

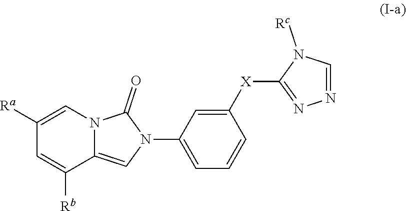

or pharmaceutically acceptable salts thereof,
   wherein
X is an optionally substituted C₁-C₃ alkylene chain, wherein one or more methylene units is optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic;
R^{a} is selected from the group consisting of —CH₂-A, —CH₂N(H)-A, and —CH₂NH₂;
R^{b} is halogen or optionally substituted C₁-C₃ alkyl;
each R^{c} is optionally substituted C₁-C₃ alkyl;
A is optionally substituted C₃-C₇ carbocylyl or optionally substituted 3-6 membered heterocyclyl containing 1-4 heteroatoms each selected from the group consisting of N, O, and S, wherein A is optionally substituted with 1-5 instances of R^{a};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S,
wherein two instances of R^{a1} are optionally taken together to form an optionally substituted C₃-C₇ carbocylyl or optionally substituted 3-6 membered heterocyclyl containing 1-4 heteroatoms each selected from the group consisting of N, O, and S;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S; or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S; and
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes a compound of formula (I-b):

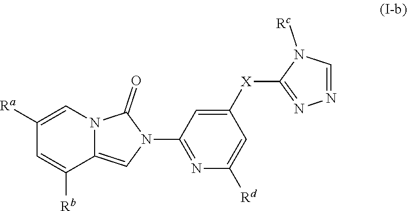

or pharmaceutically acceptable salts thereof,
   wherein
X is an optionally substituted C₁-C₃ alkylene chain, wherein one or more methylene units is optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic;
R^{a} is selected from the group consisting of —CH₂-A, —CH₂N(H)-A, and —CH₂NH₂;
R^{b} is halogen or optionally substituted C₁-C₃ alkyl;
each R^{c} is optionally substituted C₁-C₃ alkyl;
R^{d} is selected from —NR¹R² or hydrogen;
A is optionally substituted C₃-C₇ carbocylyl or optionally substituted 3-6 membered heterocyclyl containing 1-4 heteroatoms each selected from the group consisting of N, O, and S, wherein A is optionally substituted with 1-5 instances of R^{a};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S,
wherein two instances of R^{a1} are optionally taken together to form an optionally substituted C₃-C₇ carbocylyl or optionally substituted 3-6 membered heterocyclyl containing 1-4 heteroatoms each selected from the group consisting of N, O, and S;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S; and
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

### X

In some embodiments, X is an optionally substituted C₁-C₃ alkylene chain, wherein one or more methylene units is optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic. In some embodiments, X is optionally substituted C₁-C₂ alkylene. In some embodiments, X is optionally substituted C₂ alkylene optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic. In some embodiments. X is optionally substituted C₂ alkylene, wherein one methylene unit is replaced with

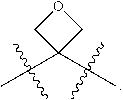 [SMILES: *C1(*)COC1]

In some embodiments, X is optionally substituted C₂ alkylene, wherein one methylene unit is replaced with

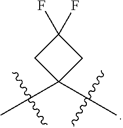 [SMILES: *C1(*)CC(F)(F)C1]

In some embodiments, X is optionally substituted C₂ alkylene, wherein one methylene unit is replaced with

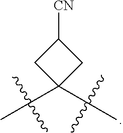 [SMILES: *C1(*)CC(C#N)C1]

In some embodiments, X is optionally substituted C₂ alkylene.

In some embodiments, wherein X is selected from the group consisting of

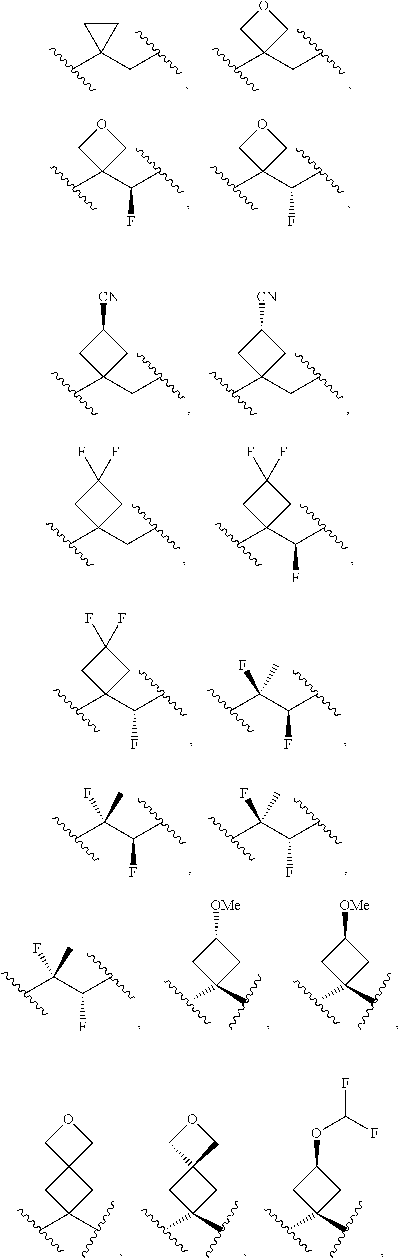

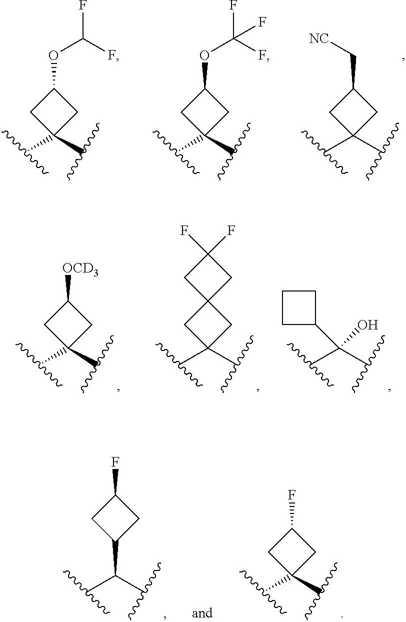

### Y

In some embodiments. Y is selected from N or C. In some embodiments, Y is N. In some embodiments, Y is C.

### R^{a}

In some embodiments, R^{a} is selected from the group consisting of —CH₂-A, —CH₂N(H)-A, and —CH₂NH₂. In some embodiments, R³ is —CH₂-A. In some embodiments, R^{a} is CH₂N(H)-A. In some embodiments, R^{a} is —CH₂NH₂.

In some embodiments. R^{a} is selected from the group consisting of —CH₂NH₂,

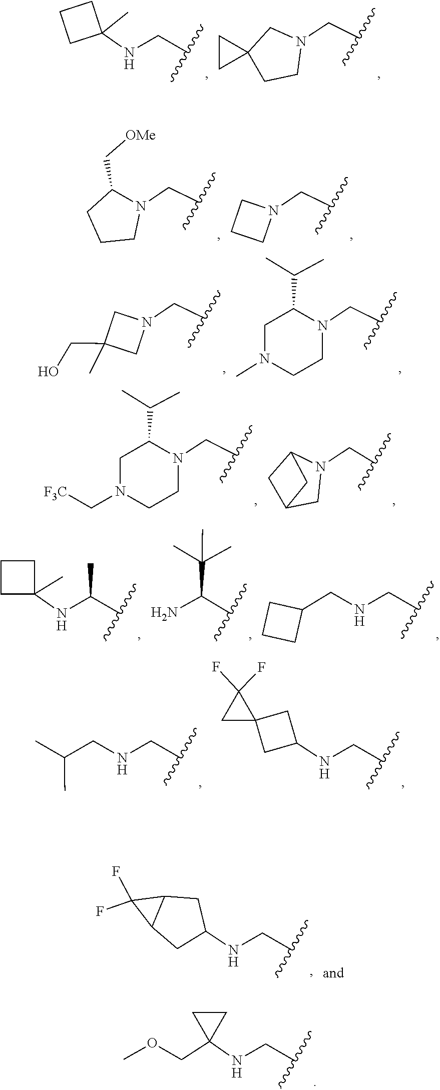

### A

In some embodiments, A is optionally substituted C₃-C₇ carbocylyl or optionally substituted 3-6 membered heterocyclyl containing 1-4 heteroatoms each selected from the group consisting of N. O, and S, wherein A is optionally substituted with 1-5 instances of R^{a1}. In some embodiments, A is optionally substituted 3-6 membered heterocyclyl containing 1-4 heteroatoms each selected from the group consisting of N, O, and S. In some embodiments, A is optionally substituted 3-6 membered carbocylyl.

### R^{b}

In some embodiments, R^{b} is halogen or optionally substituted C₁-C₃ alkyl. In some embodiments, R^{b} is halogen. In some embodiments, R^{b} is chloro. In some embodiments, R¹ is optionally substituted C₁ alkyl. In some embodiments, R^{b} selected from the group consisting of chloro, —CF₃, and —CHF₂.

### R^{c}

In some embodiments, R^{c} is optionally substituted C₁-C₃ alkyl. In some embodiments, R^{c} is —CH₃ or —CHF₂. In some embodiments, R^{c} is —CH₃. In some embodiments. R^{c} is —CHF₂.

### R^{d}

In some embodiments, R^{d} is selected from —NR¹R² or hydrogen. In some embodiments, R^{d} is —NR¹R². In some embodiments, R^{c} is hydrogen. In some embodiments, R^{d} is

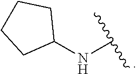 [SMILES: *NC1CCCC1]

In some embodiments, each R^{d} is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 14 heteroatoms each selected from the group consisting of N, O and S, —C(O)R³, —CO₂R³, —C(O)NHR³, and —SO₂R³. In some embodiments, each R^{d} is optionally substituted C₁-C₆ aliphatic. In some embodiments, each R^{d} is methyl.

### R²

In some embodiments, each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.

In some embodiments, each R² is optionally substituted C₁-C₆ aliphatic. In some embodiments, each R² is methyl.

### R³

In some embodiments, each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes compounds described in Table 1.

TABLE 1

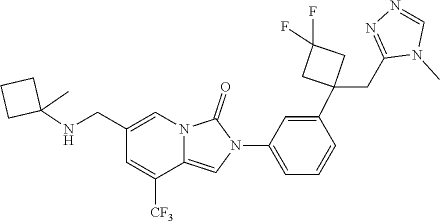 [SMILES: Cn1cnnc1CC1(c2cccc(-n3cc4c(C(F)(F)F)cc(CNC5(C)CCC5)cn4c3=O)c2)CC(F)(F)C1]

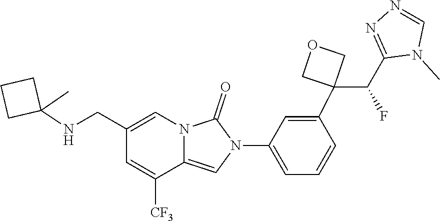 [SMILES: Cn1cnnc1[C@H](F)C1(c2cccc(-n3cc4c(C(F)(F)F)cc(CNC5(C)CCC5)cn4c3=O)c2)COC1]

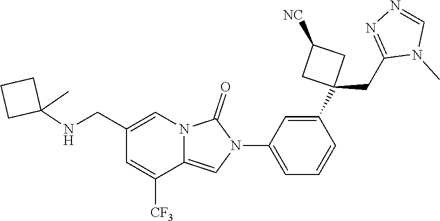

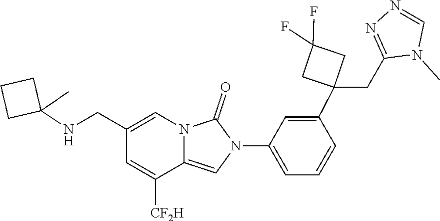 [SMILES: Cn1cnnc1CC1(c2cccc(-n3cc4c(C(F)F)cc(CNC5(C)CCC5)cn4c3=O)c2)CC(F)(F)C1]

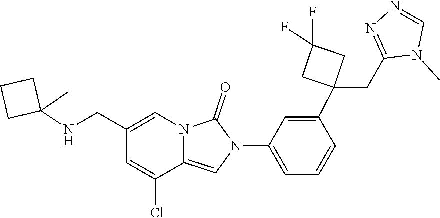 [SMILES: Cn1cnnc1CC1(c2cccc(-n3cc4c(Cl)cc(CNC5(C)CCC5)cn4c3=O)c2)CC(F)(F)C1]

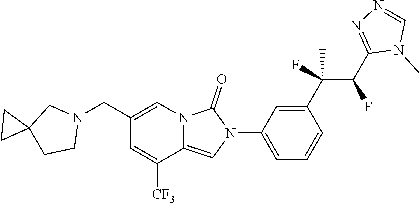 [SMILES: Cn1cnnc1[C@@H](F)[C@](C)(F)c1cccc(-n2cc3c(C(F)(F)F)cc(CN4CCC5(CC5)C4)cn3c2=O)c1]

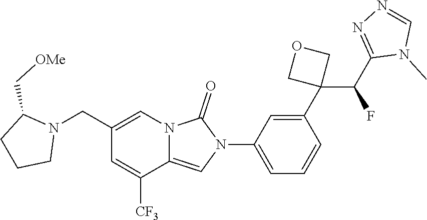 [SMILES: COC[C@H]1CCCN1Cc1cc(C(F)(F)F)c2cn(-c3cccc(C4([C@H](F)c5nncn5C)COC4)c3)c(=O)n2c1]

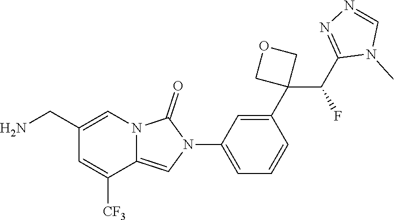 [SMILES: Cn1cnnc1[C@H](F)C1(c2cccc(-n3cc4c(C(F)(F)F)cc(CN)cn4c3=O)c2)COC1]

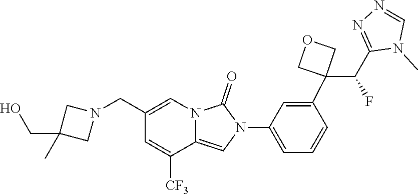 [SMILES: Cn1cnnc1[C@H](F)C1(c2cccc(-n3cc4c(C(F)(F)F)cc(CN5CC(C)(CO)C5)cn4c3=O)c2)COC1]

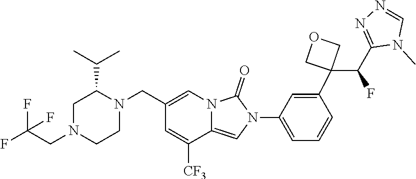 [SMILES: CC(C)[C@H]1CN(CC(F)(F)F)CCN1Cc1cc(C(F)(F)F)c2cn(-c3cccc(C4([C@H](F)c5nncn5C)COC4)c3)c(=O)n2c1]

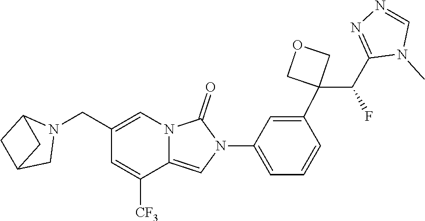 [SMILES: Cn1cnnc1[C@H](F)C1(c2cccc(-n3cc4c(C(F)(F)F)cc(CN5CC6CC5C6)cn4c3=O)c2)COC1]

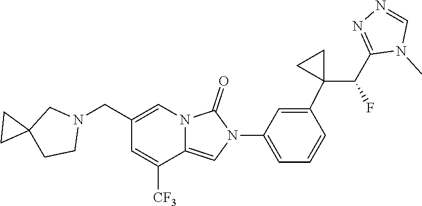 [SMILES: Cn1cnnc1[C@H](F)C1(c2cccc(-n3cc4c(C(F)(F)F)cc(CN5CCC6(CC6)C5)cn4c3=O)c2)CC1]

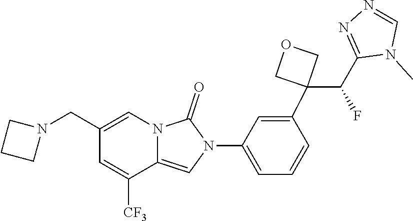 [SMILES: Cn1cnnc1[C@H](F)C1(c2cccc(-n3cc4c(C(F)(F)F)cc(CN5CCC5)cn4c3=O)c2)COC1]

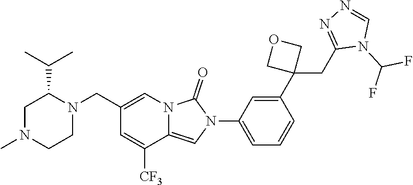 [SMILES: CC(C)[C@H]1CN(C)CCN1Cc1cc(C(F)(F)F)c2cn(-c3cccc(C4(Cc5nncn5C(F)F)COC4)c3)c(=O)n2c1]

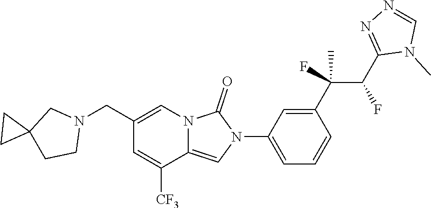 [SMILES: Cn1cnnc1[C@H](F)[C@](C)(F)c1cccc(-n2cc3c(C(F)(F)F)cc(CN4CCC5(CC5)C4)cn3c2=O)c1]

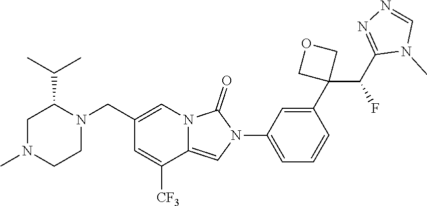

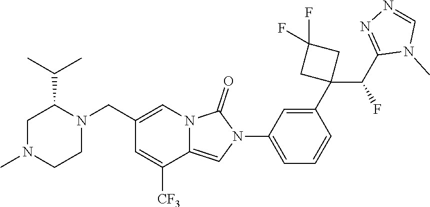

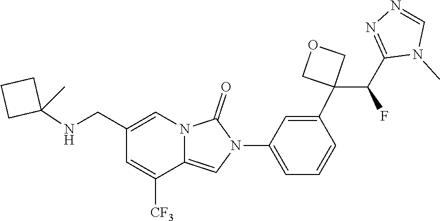 [SMILES: Cn1cnnc1[C@@H](F)C1(c2cccc(-n3cc4c(C(F)(F)F)cc(CNC5(C)CCC5)cn4c3=O)c2)COC1]

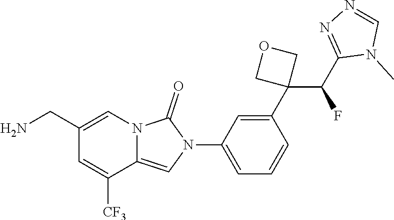 [SMILES: Cn1cnnc1[C@@H](F)C1(c2cccc(-n3cc4c(C(F)(F)F)cc(CN)cn4c3=O)c2)COC1]

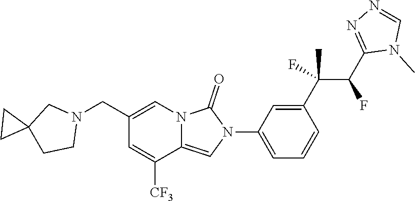 [SMILES: Cn1cnnc1[C@@H](F)[C@@](C)(F)c1cccc(-n2cc3c(C(F)(F)F)cc(CN4CCC5(CC5)C4)cn3c2=O)c1]

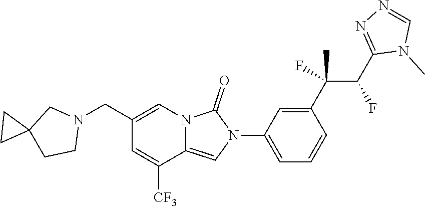 [SMILES: Cn1cnnc1[C@H](F)[C@@](C)(F)c1cccc(-n2cc3c(C(F)(F)F)cc(CN4CCC5(CC5)C4)cn3c2=O)c1]

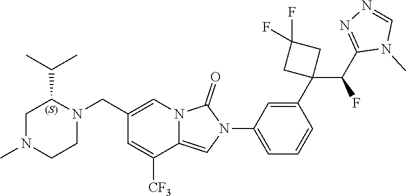

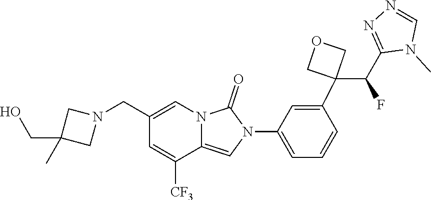 [SMILES: Cn1cnnc1[C@@H](F)C1(c2cccc(-n3cc4c(C(F)(F)F)cc(CN5CC(C)(CO)C5)cn4c3=O)c2)COC1]

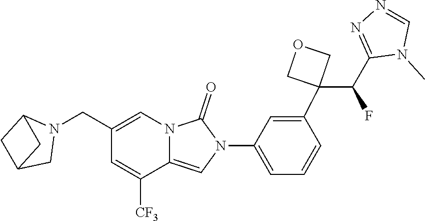 [SMILES: Cn1cnnc1[C@@H](F)C1(c2cccc(-n3cc4c(C(F)(F)F)cc(CN5CC6CC5C6)cn4c3=O)c2)COC1]

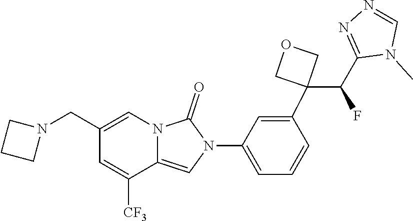 [SMILES: Cn1cnnc1[C@@H](F)C1(c2cccc(-n3cc4c(C(F)(F)F)cc(CN5CCC5)cn4c3=O)c2)COC1]

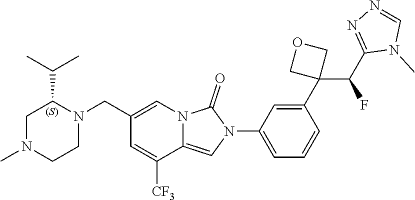

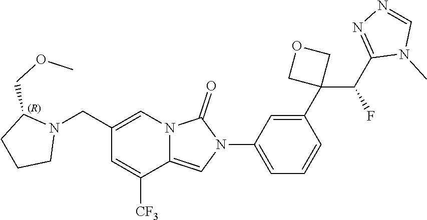

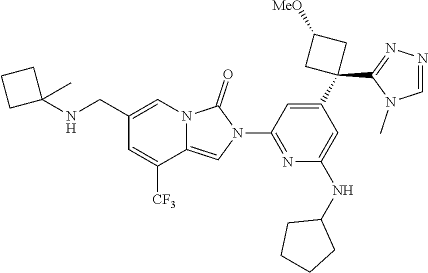 [SMILES: CO[C@H]1C[C@](c2cc(NC3CCCC3)nc(-n3cc4c(C(F)(F)F)cc(CNC5(C)CCC5)cn4c3=O)c2)(c2nncn2C)C1]

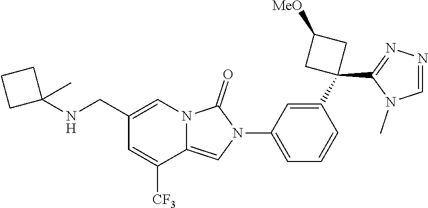 [SMILES: CO[C@H]1C[C@@](c2cccc(-n3cc4c(C(F)(F)F)cc(CNC5(C)CCC5)cn4c3=O)c2)(c2nncn2C)C1]

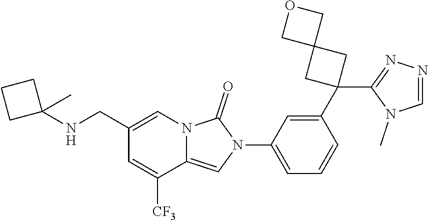 [SMILES: Cn1cnnc1C1(c2cccc(-n3cc4c(C(F)(F)F)cc(CNC5(C)CCC5)cn4c3=O)c2)CC2(COC2)C1]

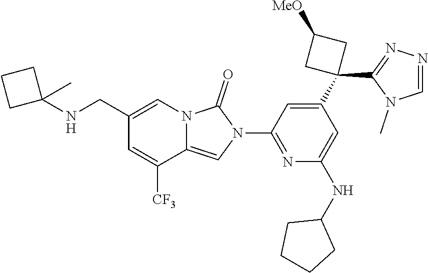 [SMILES: CO[C@H]1C[C@@](c2cc(NC3CCCC3)nc(-n3cc4c(C(F)(F)F)cc(CNC5(C)CCC5)cn4c3=O)c2)(c2nncn2C)C1]

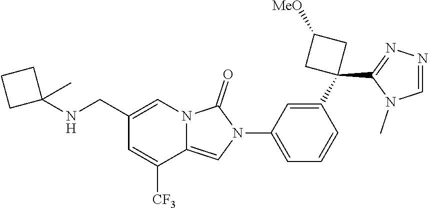 [SMILES: CO[C@H]1C[C@](c2cccc(-n3cc4c(C(F)(F)F)cc(CNC5(C)CCC5)cn4c3=O)c2)(c2nncn2C)C1]

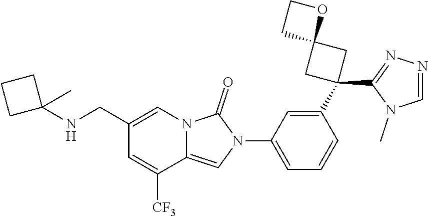 [SMILES: Cn1cnnc1[C@]1(c2cccc(-n3cc4c(C(F)(F)F)cc(CNC5(C)CCC5)cn4c3=O)c2)C[C@@]2(CCO2)C1]

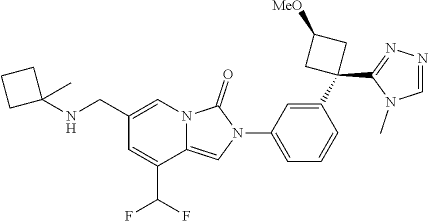 [SMILES: CO[C@H]1C[C@@](c2cccc(-n3cc4c(C(F)F)cc(CNC5(C)CCC5)cn4c3=O)c2)(c2nncn2C)C1]

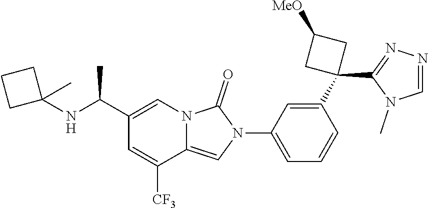 [SMILES: CO[C@H]1C[C@@](c2cccc(-n3cc4c(C(F)(F)F)cc([C@H](C)NC5(C)CCC5)cn4c3=O)c2)(c2nncn2C)C1]

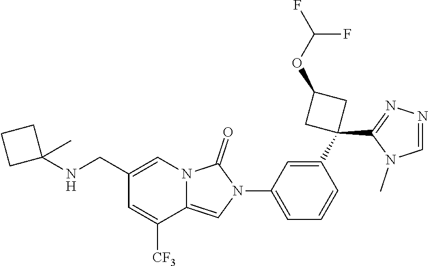 [SMILES: Cn1cnnc1[C@]1(c2cccc(-n3cc4c(C(F)(F)F)cc(CNC5(C)CCC5)cn4c3=O)c2)C[C@H](OC(F)F)C1]

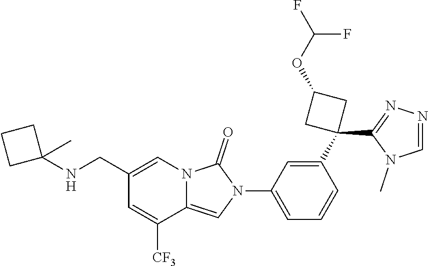 [SMILES: Cn1cnnc1[C@]1(c2cccc(-n3cc4c(C(F)(F)F)cc(CNC5(C)CCC5)cn4c3=O)c2)C[C@@H](OC(F)F)C1]

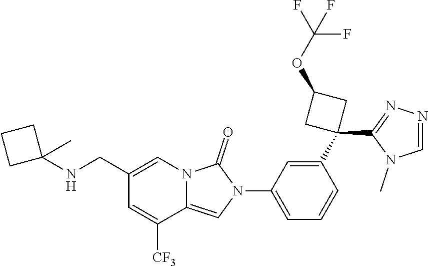 [SMILES: Cn1cnnc1[C@]1(c2cccc(-n3cc4c(C(F)(F)F)cc(CNC5(C)CCC5)cn4c3=O)c2)C[C@H](OC(F)(F)F)C1]

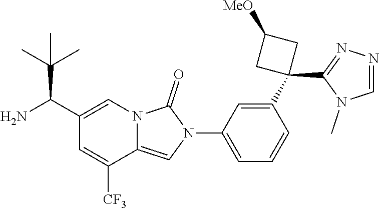 [SMILES: CO[C@H]1C[C@@](c2cccc(-n3cc4c(C(F)(F)F)cc([C@@H](N)C(C)(C)C)cn4c3=O)c2)(c2nncn2C)C1]

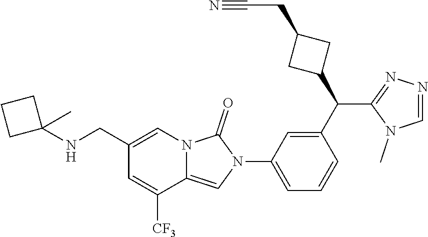

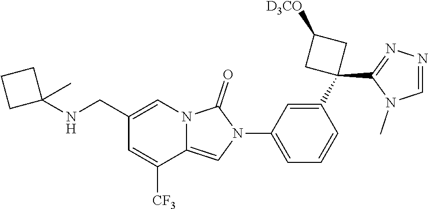

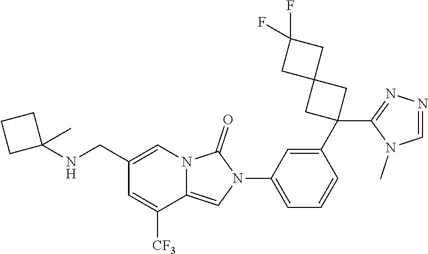 [SMILES: Cn1cnnc1C1(c2cccc(-n3cc4c(C(F)(F)F)cc(CNC5(C)CCC5)cn4c3=O)c2)CC2(CC(F)(F)C2)C1]

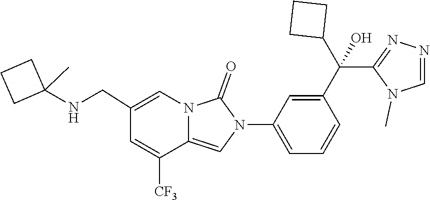 [SMILES: Cn1cnnc1[C@@](O)(c1cccc(-n2cc3c(C(F)(F)F)cc(CNC4(C)CCC4)cn3c2=O)c1)C1CCC1]

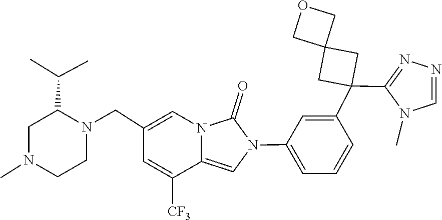

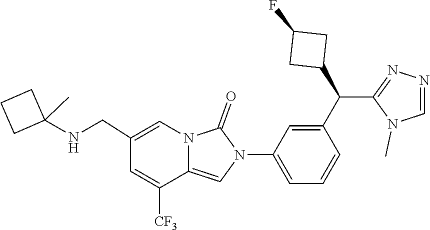

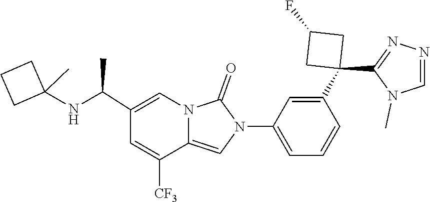 [SMILES: C[C@H](NC1(C)CCC1)c1cc(C(F)(F)F)c2cn(-c3cccc([C@]4(c5nncn5C)C[C@H](F)C4)c3)c(=O)n2c1]

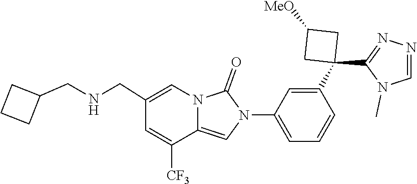 [SMILES: CO[C@H]1C[C@](c2cccc(-n3cc4c(C(F)(F)F)cc(CNCC5CCC5)cn4c3=O)c2)(c2nncn2C)C1]

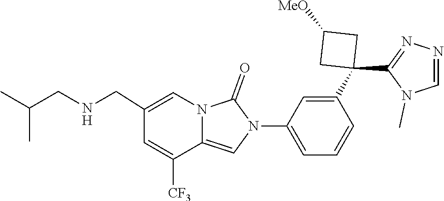 [SMILES: CO[C@H]1C[C@](c2cccc(-n3cc4c(C(F)(F)F)cc(CNCC(C)C)cn4c3=O)c2)(c2nncn2C)C1]

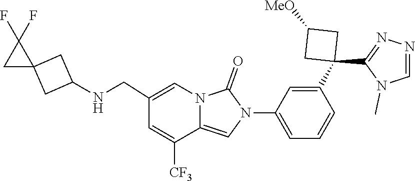 [SMILES: CO[C@H]1C[C@](c2cccc(-n3cc4c(C(F)(F)F)cc(CNC5CC6(C5)CC6(F)F)cn4c3=O)c2)(c2nncn2C)C1]

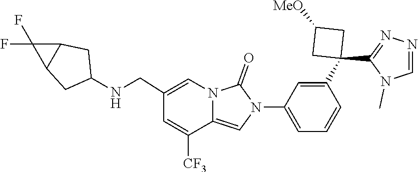 [SMILES: CO[C@H]1C[C@](c2cccc(-n3cc4c(C(F)(F)F)cc(CNC5CC6C(C5)C6(F)F)cn4c3=O)c2)(c2nncn2C)C1]

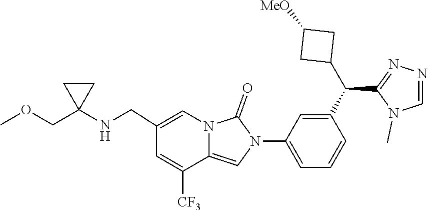

or a pharmaceutically acceptable salt thereof

A person of skill in the art will understand the present disclosure includes compounds with the stereochemistry which are the opposite of how they have been drawn. Additionally, the present disclosure contemplates tautomers of the compounds as drawn herein.

The present disclosure includes the racemate of any compound disclosed herein.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 14 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14×electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position.

Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{○}; —(CH₂)₀₋₄₀R^{○}; —O(CH₂)₀₋₄R^{○}, —O—(CH₂)₀₋₄C(O)OR^{○}; —(CH₂)₀₋₄CH(OR^{○})₂; —(CH₂)₀₋₄SR^{○}; —(CH₂)₀₋₄Ph, which may be substituted with R^{○}; —(CH₂)₀₋₄O(CH₂)₀₋₁Ph which may be substituted with R^{○}; —CH═CHPh, which may be substituted with R^{○}; —(CH₂)₀₋₄O(CH₂)i-pyridyl which may be substituted with R^{○}; —NO₂; —CN; —N₃; —(CH₂)₀₋₄N(R^{○})₂; —(CH₂)₀₋₄N(R^{○})C(O)R^{○}; —N(R^{○})C(S)R^{○}; —(CH₂)₀₋₄N(R^{○})C(O)NR^{○}₂; —N(R^{○})C(S)NR^{○}₂; —(CH₂)₀₋₄N(R^{○})C(O)OR^{○}; —N(R^{○})N(R^{○})C(O)R^{○}; —N(R^{○})N(R^{○})C(O)NR^{○}₂; —N(R^{○})N(R^{○})C(O)OR^{○}; —(CH₂)₀₋₄C(O)R^{○}; —C(S)R^{○}; —(CH₂)₀₋₄C(O)OR^{○}; —(CH₂)₀₋₄C(O)SR^{○}; —(CH₂)₀₋₄C(O)OSiR^{○}₃; —(CH₂)₀₋₄OC(O)R^{○}; —OC(O)(CH₂)₀₋₄SR^{○}, SC(S)SR^{○}; —(CH₂)₀₋₄SC(O)R^{○}; —(CH₂)₀₋₄C(O)NR^{○}₂; —C(S)NR^{○}₂; —C(S)SR^{○}; —SC(S)SR^{○}, —(CH₂)₀₋₄OC(O)NR^{○}₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂NR^{○}₂; —(CH₂)₀₋₄S(O)R^{○}; —N(R^{○})S(O)₂NR^{○}₂; —N(R^{○})S(O)₂R^{○}; —N(OR^{○})R^{○}; —C(NH)NR^{○}₂; —P(O)₂R^{○}; —P(O)R^{○}₂; —OP(O)R^{○}₂; —OP(O)(OR^{○})₂; SiR^{○}₃; —(C₁₋₄ straight or branched alkylene)O—N(R^{○})₂; or —(C₁₋₄ straight or branched alkylene)C(O)O—N(R^{○})₂, wherein each R^{○} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{○} (or the ring formed by taking two independent occurrences of R^{○} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR¹, —(CH₂)₀₋₂SH, —(CH₂)₀₋₂NH₂, —(CH₂)₀₋₂NHR^{•}, —(CH₂)₀₋₂NR^{○}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{○} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₄ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}2, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (Tor tritium); carbon may be, for example, ³C or ¹⁴C; oxygen may be, for example, ¹⁸O; nitrogen may be, for example, ¹⁵N, and the like. In other embodiments, a particular isotope (e.g., ³H, ¹³C, ⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water.

Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

The presently disclosed compounds can be formulated into pharmaceutical compositions along with a pharmaceutically acceptable carrier or excipient. According to this aspect, there is provided a pharmaceutical composition comprising a compound of Formula (I) in association with a pharmaceutically acceptable excipient, diluent or carrier.

The formulations of Compounds of Formula (T) include those suitable for the administration routes detailed herein. They may conveniently be presented in unit dosage form and can be formulated in accordance with standard pharmaceutical practice as a pharmaceutical composition. Techniques and formulations generally and suitable for use herein are found in Remington's Pharmaceutical Sciences (16^{th} edition, Osol, A. Ed. (1980); Mack Publishing Co., Easton, PA). Such methods include the step of bringing into association the active ingredient with the excipient or carrier which constitutes one or more accessory ingredients. In general the formulations are prepared by uniformly and intimately bringing into association the active ingredient with liquid excipients or carriers or finely divided solid excipients or carriers or both, and then, if necessary, shaping the product.

A typical formulation is prepared by mixing a compound of Formula (I), and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like.

The particular carrier, diluent or excipient used will depend upon the means and purpose for which the compound of Formula (I), is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of Formula (I), or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formula (I), or stabilized form of the Compound of Formula (I), (e.g., complex with a cyclodextrin derivative or other known complexation agent) is dissolved in a suitable solvent in the presence of one or more of the excipients described above. The compound of Formula (I) is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations may be prepared for various routes and types of administration. For example, a compound of Formula (I) having the desired degree of purity may optionally be mixed with pharmaceutically acceptable diluents, carriers, excipients or stabilizers (Remington's Pharmaceutical Sciences (1980) 16^{th} edition, Osol, A. Ed.), in the form of a lyophilized formulation, milled powder, or an aqueous solution.

Formulation may be conducted by mixing at ambient temperature at the appropriate pH, and at the desired degree of purity, with physiologically acceptable excipients or carriers. i.e., excipients or carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compounds of Formula (I) can be sterile. In particular, formulations to be used for in vivo administration should be sterile. Such sterilization is readily accomplished by filtration through sterile filtration membranes.

The compound of Formula (I) ordinarily can be stored as a solid composition, a lyophilized formulation or as an aqueous solution.

The pharmaceutical compositions comprising a compound of Formula (I) can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. In some embodiments, the amount is below the amount that is toxic to the host or renders the host more susceptible to bleeding.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and in-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEO). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations of Formula (I) compounds may be prepared. Suitable examples of sustained-release preparations include semipermeable matrices of solid hydrophobic polymers containing a compound of Formula (I), which matrices are in the form of shaped articles, e.g., films, or microcapsules. Examples of sustained-release matrices include polyesters, hydrogels (for example, poly(2-hydroxyethyl-methacrylate), or poly(vi nyl alcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

Formulations of a compound of Formula (I) suitable for oral administration may be prepared as discrete units such as pills, capsules, cachets or tablets each containing a predetermined amount of a compound of Formula (I).

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formula (I) intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400), and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise solely an emulsifier, it may also comprise a mixture of at least one emulsifier and a fat or oil, or both a fat and an oil. A hydrophilic emulsifier included together with a lipophilic emulsifier may act as a stabilizer. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation include Tween® 60, Span® 80, cetostearyl alcohol, benzyl alcohol, myristyl alcohol, glyceryl mono-stearate and sodium lauryl sulfate.

Aqueous suspensions of Formula (I) compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formula (I), may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the excipient or carrier material to produce a single dosage form will vary depending upon the host treated and the particular mode of administration. For example, a time-release formulation intended for oral administration to humans may contain approximately 1 to 1000 mg of active material compounded with an appropriate and convenient amount of excipient or carrier material which may vary from about 5 to about 95% of the total compositions (weightweight). The pharmaceutical composition can be prepared to provide easily measurable amounts for administration. For example, an aqueous solution intended for intravenous infusion may contain from about 3 to 500 pg of the active ingredient per milliliter of solution in order that infusion of a suitable volume at a rate of about 30 mU/hr can occur.

Formulations suitable for parenteral administration include aqueous and nonaqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops in which the active ingredient is dissolved or suspended in a suitable excipient or carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of disorders as described below.

Formulations suitable for vaginal administration may be presented as pessaries, tampons, creams, gels, pastes, foams or spray formulations containing in addition to the active ingredient such excipients or carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid excipient or carrier, for example water, for injection immediately prior to use. Extemporaneous injection solutions and suspensions are prepared from sterile powders, granules and tablets of the kind previously described.

Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In particular embodiments the pharmaceutical composition comprising the presently disclosed compounds further comprise a chemotherapeutic agent. In some of these embodiments, the chemotherapeutic agent is an immunotherapeutic agent.

### Kits

Further provided are kits for carrying out the methods detailed herein, which kits comprise one or more compounds described herein or a pharmaceutical composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a disorder such as cancer. In some embodiments, the kit contains instructions for use in the treatment of a cancer.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disorder (e.g., cancer) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and may be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a subject.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is hyperplasia or cancer. In some embodiments, a disease or condition associated with cell proliferation is cancer.

In some embodiments, administration of a compound of the present disclosure results in arrest of mitosis.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is a hematologic cancer. In some embodiments, a hematologic cancer is selected from a group consisting of lymphoma, leukemia, and myeloma. In some embodiments, a hematologic cancer is lymphoma. In some embodiments, a hematologic cancer is leukemia. In some embodiments, a hematologic cancer is myeloma.

In some embodiments, cancer is a non-hematologic cancer. In some embodiments, a non-hematologic cancer is a sarcoma or a carcinoma. In some embodiments, a non-hematologic cancer is a sarcoma. In some embodiments, a non-hematologic cancer is carcinoma.

In some embodiments, a subject has one or more of increased T-cell activation, increased T-cell proliferation, decreased T-cell exhaustion, decreased T-cell anergy and decreased T-cell tolerance after administration of compound of the present disclosure. In some embodiments, administration of a compound of the present disclosure to a subject in need there of results in one or more of increased T-cell activation, increased T-cell proliferation, decreased T-cell exhaustion, decreased T-cell anergy and decreased T-cell tolerance.

In some embodiments, a subject has increased NK-cell activation. In some embodiments, increased NK-cell activation comprises increased production of cytokines.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

The presently disclosed compounds find use in inhibiting the activity of Cbl-B. Many of the compounds additionally do so with an inhibitory effect that is greater than that for C-cbl.

In one embodiment, the subject matter disclosed herein is directed to a method of inhibiting Cbl-B, the method comprising contacting one or more cells containing active Cbl-B proteins with an effective amount of a compound of Formula (T), or a pharmaceutical composition described herein. By “contacting” is meant bringing the compound within close enough proximity to an isolated Cbl-B enzyme or a cell expressing Cbl-B (e.g., T cell, B cell, dendritic cell) such that the compound is able to bind to and inhibit the activity of Cbl-B. The compound can be contacted with Cbl-B in vitro or in vivo via administration of the compound to a subject.

In an embodiment, the subject matter disclosed herein is directed to a method for enhancing an immune response in a subject in need thereof, wherein the method comprises administering to said subject an effective amount of a compound of Formula (I)), or a pharmaceutical composition described herein. In certain aspects of this embodiment, the T cells in the subject have at least one of enhanced priming, enhanced activation, enhanced migration, enhanced proliferation, enhanced survival, and enhanced cytolytic activity relative to prior to the administration of the compound or pharmaceutical composition. In certain aspects of this embodiment, the T cell activation is characterized by an elevated frequency of y-IFN+CDS T cells, an elevated frequency of y-IFN+CD4 T cells, or enhanced levels of IL-2 or granzyme B production by T cells, relative to prior to administration of the compound or pharmaceutical composition. In certain aspects of this embodiment, the number of T cells is elevated relative to prior to administration of the compound or pharmaceutical composition.

In certain aspects of this embodiment, the T cell is an antigen-specific CDS T cell. In certain aspects of this embodiment, the T cell is an antigenspecific CD4 T cell. In certain aspects of this embodiment, the antigen presenting cells in the subject have enhanced maturation and activation relative prior to the administration of the compound or pharmaceutical composition. In certain aspects of this embodiment, the antigen presenting cells are dendritic cells. In certain aspects of this embodiment, the maturation of the antigen presenting cells is characterized by increased frequency of CD83+dendritic cells. In certain aspects of this embodiment, the activation of the antigen presenting cells is characterized by elevated expression of CD80 and CD86 on dendritic cells. In some aspects, compounds of Formula (I), or variations thereof, or a pharmaceutical composition thereof provides general priming of the immune response (i.e., vaccines) to tumors or viruses for boosting/generating anti-viral/tumor immunity.

In another embodiment, the subject matter disclosed herein is directed to a method for treating a cancer, the method comprising administering to a subject in need thereof an effective amount of a compound of Formula (I), or a pharmaceutical composition thereof as further described herein. It is understood that the compound functions by inhibiting Cbl-B in a manner that leads to activated T cells that are able to kill cancer cells, regardless of their origin in the body. In certain aspects of this embodiment, the cancer comprises at least one cancer selected from the group consisting of colorectal cancer, melanoma, non-small cell lung cancer, ovarian cancer, breast cancer, pancreatic cancer, a hematological malignancy, and a renal cell carcinoma. In certain aspects of this embodiment, the cancer has elevated levels of T-cell infiltration. In certain aspects of this embodiment, the cancer cells in the subject selectively have elevated expression of MHC class I antigen expression relative to prior to the administration of the compound or composition.

In the methods described herein, the method can further comprise administering a therapeutic, or chemotherapeutic agent to said subject. For example, such an agent may be an inhibitor of PD-L1/PD-1. In certain aspects of this embodiment, the therapeutic or chemotherapeutic agent is administered to the subject simultaneously with the compound or the composition. In certain aspects of this embodiment, the therapeutic or chemotherapeutic agent is administered to the subject prior to administration of the compound or the composition. In certain aspects of this embodiment, the therapeutic or chemotherapeutic agent is administered to the subject after administration of the compound or said composition.

As used herein, “enhancing an immune response” refers to an improvement in any immunogenic response to an antigen. Non-limiting examples of improvements in an immunogenic response to an antigen include enhanced maturation or migration of dendritic cells, enhanced activation of T cells (e.g., CD4 T cells. CDS T cells), enhanced T cell (e.g., CD4 T cell, CDS T cell) proliferation, enhanced B cell proliferation, increased survival of T cells and/or B cells, improved antigen presentation by antigen presenting cells (e.g., dendritic cells), improved antigen clearance, increase in production of cytokines by T cells (e.g., interleukin-2), increased resistance to prostaglandin E2-induced immune suppression, and enhanced priming and/or cytolytic activity of CDS T cells.

In some embodiments, the CDS T cells in the subject have enhanced priming, activation, proliferation and/or cytolytic activity relative to prior to the administration of a compound of Formula (I), or a pharmaceutically acceptable salt, prodrug, metabolite, or derivative thereof. In some embodiments, the CDS T cell priming is characterized by elevated CD44 expression and/or enhanced cytolytic activity in CDS T cells. In some embodiments, the CDS T cell activation is characterized by an elevated frequency of y-1 FN+CDS T cells. In some embodiments, the CDS T cell is an antigenspecific T-cell.

In some embodiments, the CD4 T cells in the subject have enhanced priming, activation, proliferation and/or cytolytic activity relative to prior to the administration of the compound of Formula (I), or a pharmaceutically acceptable salt, prodrug, metabolite, or derivative thereof. In some embodiments, the CD4 T cell priming is characterized by elevated CD44 expression and/or enhanced cytolytic activity in CD4 T cells. In some embodiments, the CD4 T cell activation is characterized by an elevated frequency of y-IFN+CD4 T cells. In some embodiments, the CD4 T cell is an antigenspecific T-cell.

Accordingly, the presently disclosed compounds of Formula (I), or pharmaceutically acceptable salts, prodrugs, metabolites, or derivatives thereof are useful in treating T cell dysfunctional disorders. A “T cell dysfunctional disorder” is a disorder or condition of T cells characterized by decreased responsiveness to antigenic stimulation.

Thus, the presently disclosed compounds can be used in treating conditions where enhanced immunogenicity is desired, such as increasing tumor immunogenicity for the treatment of cancer.

“Immunogenicity” refers to the ability of a particular substance to provoke an immune response. Tumors are immunogenic and enhancing tumor immunogenicity aids in the clearance of the tumor cells by the immune response. Viruses may also be immunogenic and enhancing/activating immunogenicity may aid in clearance of viral particles by the immune response.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The compounds herein may be used in conjunction with one or more chemotherapeutic agents, in the course of treating a patient. A “chemotherapeutic agent” is a chemical compound or biologic useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARI NOLO); beta-lapachone; lapachol; colchicines; betulinic acid; a camptothecin (including the synthetic analogue topotecan (HYCAMTIN®), CPT-1l (irinotecan, CAMPTOSA®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyl li nic acid; teniposide; cryptophycins (particularly cryptophycin 1 and cryptophycin 8); dolastatin; duocarmycin (including the synthetic analogues. KW-2189 and CBI-TMI); eleutherobin; pa ncrati statin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see. e.g., Nicolaou et al., Angew. Chem Inti. Ed. Engl., 33: 183-186 (1994)); dynemicin, including dynemicin A; an esperamicin; as well as neocarzi nostatin chromophore and related chromoprotein enediyne antibiotic chromophores), streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene. OR); razoxane; rhizoxin; sizotiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorornetlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents that can be deployed in treatment protocols that involve the Cbl-B inhibitor compounds herein, include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene. LYII 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tri pterelin; anti-androgens such as flutamide, nilutamide and bicalutamide; and aromatase inhibitors that inhibit the enzyme aromatase, which regulates estrogen production in the adrenal glands, such as, for example, 4 (5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DTDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, 1-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an antiestrogen such as fulvestrant; EGFR inhibitor such as erlotinib or cetuximab; an anti-VEGF inhibitor such as bevacizumab; arinotecan; rmRH (e.g., ABARELIX®); 17AAG (geldanamycin derivative that is a heat shock protein (Hsp) 90 poison), and pharmaceutically acceptable salts, acids or derivatives of any of the above.

Also included in the definition of “chemotherapeutic agent” are; anti-hormonal agents that act to regulate or inhibit hormone action on tumors such as anti-estrogens and selective estrogen receptor modulators (SERMs), including, for example, tamoxifen (including NOL V ADEX®; tamoxifen citrate), raloxifene, droloxifene, 4-hydroxytamoxifen, trioxifene, keoxifene, LYII 7018, onapristone, and FARESTON® (toremifine citrate); aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); antiandrogens such as flutamide, nilutamide, bicalutamide, leuprolide, and goserelin; as well as troxacitabine (a 1,3-dioxolane nucleoside cytosine analog); protein kinase inhibitors; lipid kinase inhibitors; antisense oligonucleotides, particularly those which inhibit expression of genes in signaling pathways implicated in aberrant cell proliferation, such as, for example, PKC-alpha, Ralf and H-Ras; ribozymes such as VEGF expression inhibitors (e.g., ANGIOZYME®) and HER2 expression inhibitors; vaccines such as gene therapy vaccines, for example, ALLOVECTIN®, LEUVECTIN®, and VAXID®; PROLEUKIN® rlL-2; a topoisomerase 1 inhibitor such as LURTOTECAN®: ABARELIX® rmRH; antiangiogenic agents such as bevacizumab (AV ASTIN®. Genentech); and pharmaceutically acceptable salts, acids and derivatives of any of the above.

In some embodiments, the chemotherapeutic agent is an immunotherapeutic agent. As used herein, an “immunotherapeutic agent” is a compound that enhances the immune system to help fight cancer, specifically or non-specifically. Immunotherapeutics include monoclonal antibodies and non-specific immunotherapies that boost the immune system, such as cytokines, interleukins (e.g., IL-2, IL-7. IL-12, IL-15, IL-21), interferons (e.g., IFN-α, IFN-˜, IFN-γ), GMCSF, thalidomide, iTHALOMID®, Celgene), lenalidomide (REVLIMID®, Celgene), pomalidomide (POMALYST®, Celgene), imiquimod (ZYCLARA®, Valeant).

Non-limiting examples of monoclonal antibodies that are useful as a chemotherapeutic agent include trastuzumab (HERCEPTIN®, Genentech), bevacizumab (AV ASTIN®, Genentech), cetuximab (ERBITUX®, Bristol-Myers Squibb), panitumumab (VECTIBIX®, Amgen), ipilimumab (YERVOY®, Bristol-Myers Squibb), rituximab (RITUXAN®, Genentech), alemtuzumab (CAMPATH®, Genzyme), ofatumumab (ARZERRA. Genmab), gemtuzumab ozogamicin (MYLOTARG®, Wyeth), brentuximab vedotin (ADCETRIS®, Seattle Genetics), 90Y-labelled ibritumomab tiuxetan (ZEVALIN®, Biogen Idee), 1311-labelled tositumomab (BEXXAR®, GlaxoSmithKline), ado-trastuzumab emtansine (KADCYLAC®). Genentech) blinatunomab (BLINCYTO®, Amgen), pertuzumab (PERJETA®, Genentech), obinutuzumab (GAZYVA®, Genentech), nivolumab (OPDIVO®) Bristol-Myers Squibb), pembrolizumah (KEYTRIUDA®, Merck), pidilizumab (CureTech), Tiragolumab (Roche/Genentech, described in WHO Drug Information (International Nonproprietary Names for Pharmaceutical Substances), Proposed INN; List 117, Vol. 31, No. 2, Jun. 9, 2017 (page 343)), MPDL3280A (Atezolizumab, Roche/Genentech), MDX-1105 (described in WO2007/005874), and MEDI4736 (IMFINZI®, Durvalumab, Medarex). Another useful immunotherapeutic agent is AMP-224 (described in WO2010/027827 and WO2011/066342).

In some embodiments, the compound is administered to the subject at a dose of between about 0.001 pg/kg and about 10.000 mg/kg, including but not limited to about 0.001 pg/kg, about 0.01 pg/kg, about 0.05 pg/kg, about 0.1 pg/kg, about 0.5 pg/kg, about 1 pg/kg, about 10 pg/kg, about 25 pg/kg, about 50 pg/kg, about 100 pg/kg, about 250 pg/kg, about 500 pg/kg, about 1 mg/kg, about 5 mg/kg, about 10 mg/kg, about 25 mg/kg, about 50 mg/kg, about 100 mg/kg, and about 20) mg/kg.

### Exemplification

### Example 1. Synthesis of Compound 1

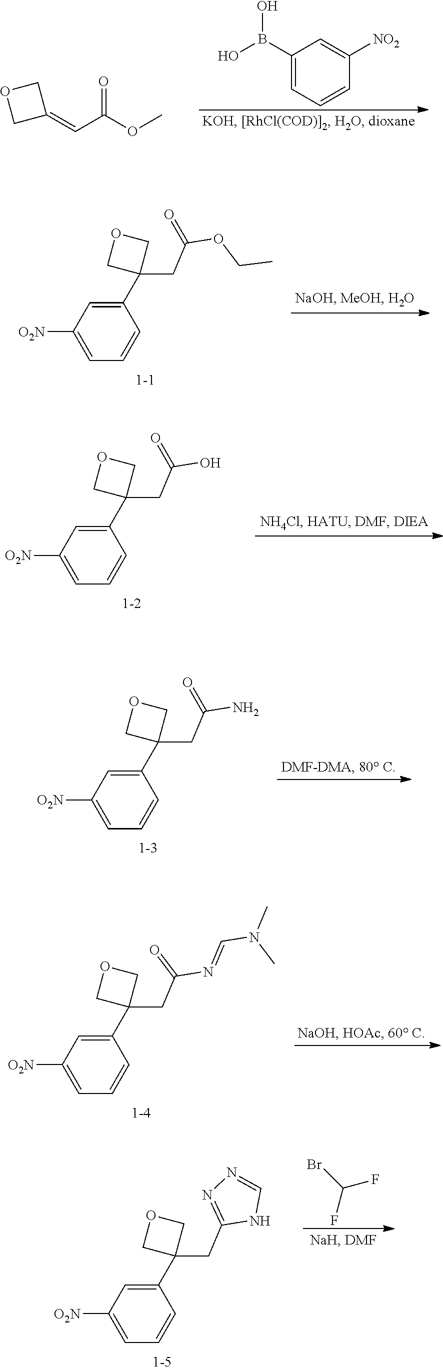

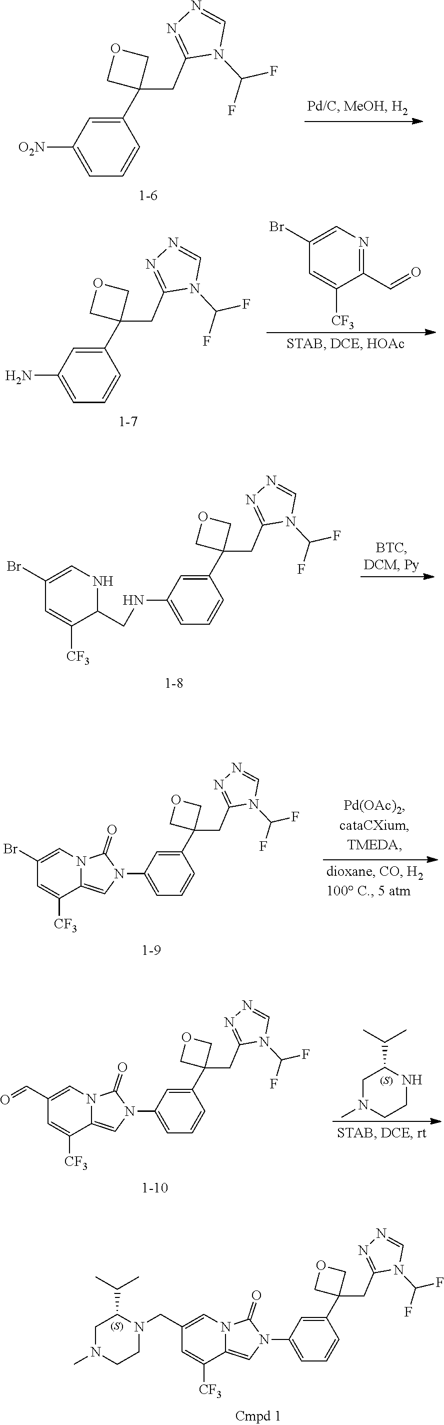

### Synthesis of 1-1

To a stirred solution of [Rh(COD)Cl]₂ (10.41 g, 21.104 mmol, 0.1 equiv) in dioxane (400 mL) was added KOH (14.21 g, 253.247 mmol, 1.2 equiv) and H₂O (100 mL) at room temperature under nitrogen atmosphere. The resulting mixture was stirred for 1 h at room temperature under nitrogen atmosphere. To the above mixture was added ethyl 2-(oxetan-3-ylidene)acetate (30 g, 211.039 mmol, 1 equiv) and 3-nitrophenylboronic acid (59.89 g, 358.766 mmol, 1.7 equiv) at room temperature. The resulting mixture was stirred overnight at 80′C. The reaction was quenched with sat. NH₄Cl (aq.) (300 mL) at room temperature. The aqueous layer was extracted with EtOAc (3×30) mL). The resulting mixture was concentrated under reduced pressure. The residue was purified by silica gel column chromatography, eluted with PE/EA (5:1) to afford 1-1 (25 g, 42.42%) as a yellow solid.

### Synthesis of 1-2

To a stirred solution of 1-1 (25 g, 99.507 nmol, 1.00 equiv) in MeOH (250 mL) was added NaOH (298.5 mL, 298.500 mmol, 3.00 equiv) at room temperature. The resulting mixture was stirred overnight at room temperature. The MeOH was removed under vacuum. The mixture was acidified to pH 4 with HCl (1 M). The precipitated solids were collected by filtration and washed with water (100 mL). The resulting solid was dried in an oven under reduced pressure. This resulted in 1-2 (20 g, 76.26%) as a white solid.

### Synthesis of 1-3

To a stirred solution of 1-2 (20 g, 84.313 mmol, 1.00 equiv) and NH₄Cl (13.53 g, 252.939 mmol, 3.00 equiv) in DMF (200 mL) was added DTEA (43.59 g, 337.252 mmol, 4.00 equiv) and HATU (48.09 g, 126.470 mmol, 1.50 equiv) at room temperature. The resulting mixture was stirred overnight at room temperature under argon atmosphere. The resulting mixture was diluted with water (400 mL). The aqueous layer was extracted with EtOAc (3×150 mL). The resulting mixture was concentrated under vacuum. The residue was purified by trituration with water (100 mL). The resulting solid was dried in an oven under reduced pressure. This resulted in 1-3 (10 g, 47.70%) as an off-white solid.

### Synthesis of 1-4

A solution of 1-3 (10 g, 42.332 mmol, 1 equiv) in DMF-DMA (100 mL) was stirred for overnight at 80° C. The resulting mixture was diluted with water (100 mL). The resulting mixture was extracted with EtOAc (3×100 mL). The combined organic layers were washed with brine (50 mL), dried over anhydrous Na₂SO₄. After filtration, the filtrate was concentrated under reduced pressure. This resulted in 1-4 (10 g, 71.36%) as a light brown solid.

### Synthesis of 1-5

To a stirred solution of 1-4 (9.9 g, 33.985 mmol, 1.00 equiv) in HOAc (100 mL) was added hydrazine hydrate (80 mL, 98%) dropwise at room temperature. The resulting mixture was stirred overnight at 60° C. The resulting mixture was diluted with water (300 mL). The aqueous layer was extracted with EtOAc (3×200 mL). The combined organic layers were being dried over anhydrous Na₂SO₄. After filtration, the filtrate was concentrated under reduced pressure. This resulted in 1-5 (5.4 g, 56.78%) as a grey solid.

### Synthesis of 1-6

To a stirred solution of 1-5 (5.3 g, 20.365 mmol, 1.00 equiv) in DMF (30.00 mL) was added NaH (4.07 g, 101.825 mmol, 5.00 equiv, 60%) in portions at 0CC under nitrogen atmosphere. The resulting mixture was stirred for 30 min at 0° C. under nitrogen atmosphere. To the above mixture was added bromodifluoromethane (159.97 g, 122.190 mmol, 6.00 equiv, 10%) at room temperature. The resulting mixture was stirred for additional overnight at room temperature. The reaction was quenched by the addition of water (500 mL). The aqueous layer was extracted with EtOAc (3×200 mL). The resulting mixture was concentrated under vacuum. The residue was purified by reverse flash chromatography with the following conditions: column. C18 silica gel; mobile phase, MeCN in water (10 mmol NH₄HCO₃), 50% to 60% gradient in 10 min; detector, UV 254 nm. This resulted in 1-6 (2 g, 30.07%) as a white solid.

### Synthesis of 1-7

To a solution of 1-6 (2 g, 6.446 mmol, 1 equiv) in 20 mL MeOH was added Pd/C (10%, 0.2 g) under nitrogen atmosphere. The mixture was hydrogenated at room temperature for overnight under hydrogen atmosphere using a hydrogen balloon, filtered through a Celite pad and concentrated under reduced pressure. This resulted in 1-7 (1.8 g, 89.66%) as a light brown solid.

### Synthesis of 1-8

To a stirred solution of 1-7 (900 mg, 3.211 mmol, 1 equiv) and 5-bromo-3-(trifluoromethyl)pyridine-2-carbaldehyde (978.76 mg, 3.853 mmol, 1.2 equiv) in DCE (10 mL) was added Acetate (192.83 mg, 3.211 mmol, 1 equiv) and NaBH(OAc)₃ (1.36 g, 6.422 mmol, 2 equiv) at room temperature. The resulting mixture was stirred overnight at room temperature. The reaction was quenched by the addition of NaHCO₃ (aq.) (100 mL) at room temperature. The aqueous layer was extracted with DCM (3×50 mL). The resulting mixture was concentrated under reduced pressure. The residue was purified by silica gel column chromatography, eluted with DCM/MeOH (20:1) to afford 1-8 (1.2 g, 68.63%) as a light yellow solid.

### Synthesis of 1-9

To a stirred solution of 1-8 (1.2 g, 2.320 mmol, 1.00 equiv) and Pyridine (1.10 g, 13.920 mmol, 6 equiv) in DCM (30 mL) was added Triphosgene (0.24 g, 0.812 mmol, 0.35 equiv) at 0° C. The resulting mixture was stirred for 30 min at room temperature. The resulting mixture was washed with NaHCO₃ (10) mL). The residue was purified by Prep-TLC (DCM/MeOH 20:1) to afford 1-9 (0 g, 74.45%) as a yellow solid.

### Synthesis of 1-10

To a stirred solution of 1-9 (1 g, 1.837 mmol, 1.00 equiv) in dioxane (30 mL) was added bis(adamantan-1-yl) (butyl)phosphane (131.75 mg, 0.367 mmol, 0.20 equiv), Pd(OAc)₂ (41.25 mg, 0.184 mmol, 0.1 equiv) and TMEDA (427.00 mg, 3.674 nmol, 2 equiv) in a pressure tank. The mixture was purged with nitrogen for 3 min and then was pressurized to 10 atm with CO at 90 degrees C. for overnight. The reaction mixture was cooled to room temperature and diluted with water. The aqueous layer was extracted with DCM (2×60 mL). The residue was purified by Prep-TLC (DCM/MeOH 20:1) to afford 1-10 (300 mg, 23.17%) as a yellow solid.

### Synthesis of Compound 1

To a stirred solution of 1-10 (270 mg, 0.547 mmol, 1 equiv) was added (3S)-3-isopropyl-1-methylpiperazine (116.76 mg, 0.821 mmol, 1.5 equiv) at room temperature. The resulting mixture was stirred for 61 h at room temperature. To the above mixture was added STAB (231.96 mg, 1.094 mmol, 2 equiv) at room temperature. The resulting mixture was stirred overnight at room temperature. The reaction was quenched with water at room temperature. The aqueous layer was extracted with DCM (2×20 mL). The resulting mixture was concentrated under vacuum. The residue was purified by reversed-phase flash chromatography with the following conditions: column. C18 silica gel; mobile phase, MeCN in Water (0.1% NH₃·H₂O), 10% to 50% gradient in 10 min; detector, UV 254 nm. This resulted in Compound 1 (60.1 mg, 17.37%) as a yellow solid.

LC-MS-Compound 1: (ES, m/z): [M+H]⁺ 620

H-NMR-Compound 1: (400 MHz, DMSO-d6, ppm): δ 0.95-1.00 (m, 3H), δ 2.11-2.16 (m, 1H), δ 2.17-2.19 (m, 11H), δ 2.28-2.32 (s, 6H), δ 2.68-2.87 (m, 2H), δ 2.87-2.89 (m, 1H), δ 2.90-2.97 (m, 1H), δ 3.81 (s, 2H), δ 3.93-3.96 (m, 1H), δ 7.02-7.06 (m, 1H), 67.21 (s, 3H), δ 7.35 (s, 2H), δ 7.42-7.45 (m, 1H), δ 7.56-7.66 (m, 1H) δ 8.66 (s, 1H).

### Example 2. Synthesis of Compound 2

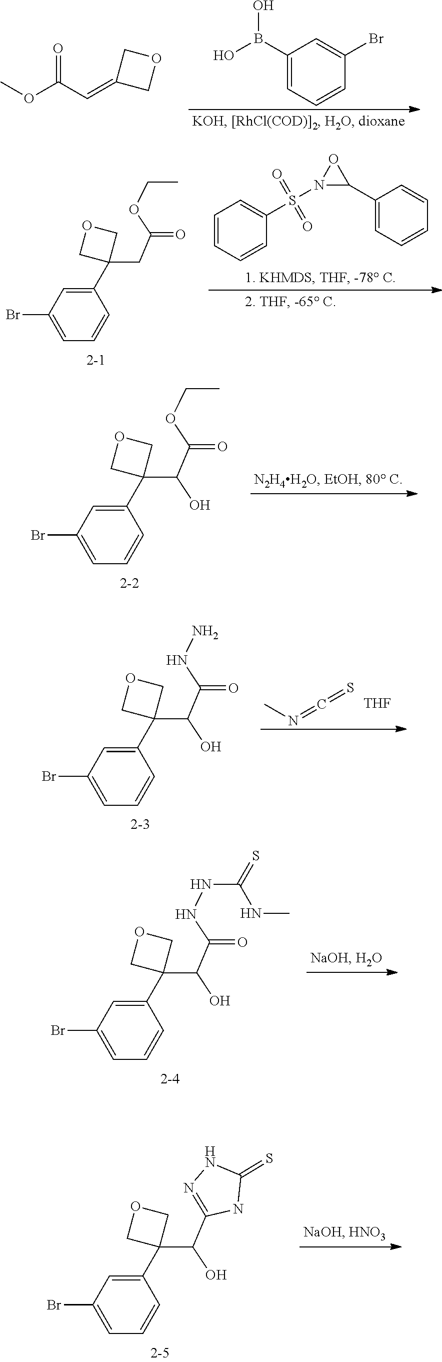

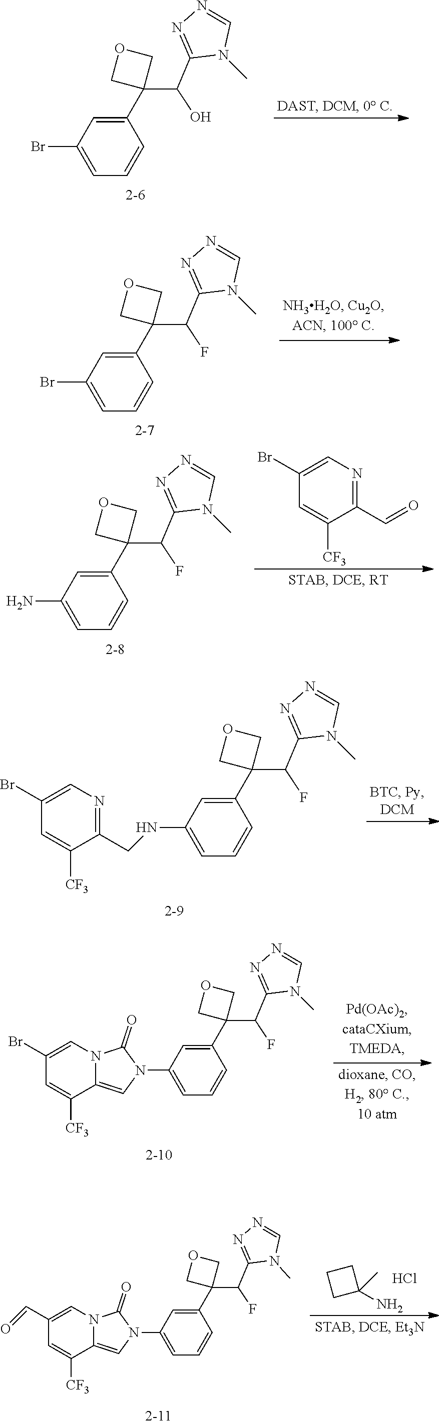

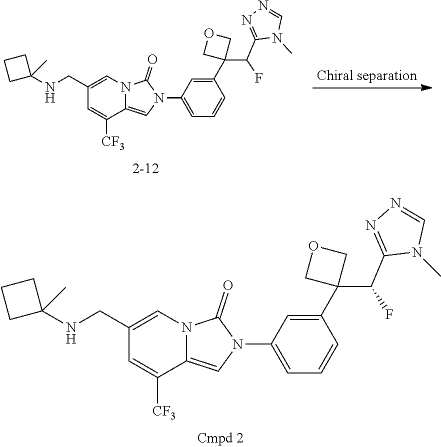 [SMILES: Cn1cnnc1C(F)C1(c2cccc(-n3cc4c(C(F)(F)F)cc(CNC5(C)CCC5)cn4c3=O)c2)COC1 ; Cn1cnnc1[C@H](F)C1(c2cccc(-n3cc4c(C(F)(F)F)cc(CNC5(C)CCC5)cn4c3=O)c2)COC1]

### Synthesis of 2-1

To a stirred solution/mixture of [Rh(COD)Cl]₂ (4.16 g, 8.442 mmol, 0.015 equiv) in dioxane (1000 mL) was added KOH (37.89 g, 675.324 mmol, 1.2 equiv and H₂O (2000 mL) at room temperature under nitrogen atmosphere. The resulting mixture was stirred for 1h at room temperature under nitrogen atmosphere. To the above mixture was added ethyl 2-(oxetan-3-ylidene)acetate (80 g, 562.770 mmol, 1.00 equiv-) and 3-bromophenylboronic acid (192.14 g, 956.709 mmol, 1.7 equiv) at room temperature. The resulting mixture was stirred for additional overnight at 80° C. The reaction was quenched with sat. NH₄Cl (aq.)(1000 mi.) at room temperature. The aqueous layer was extracted with EtOAc (3×800 mL). The resulting mixture was concentrated under reduced pressure. The residue was purified by silica gel column chromatography, eluted with PE/EA (5:1) to afford 2-1 (120 g, 65.57%) as a yellow oil.

### Synthesis of 2-2

To a stirred solution of 2-1 (120 g, 401.118 mmol, 1.00 equiv) in THF (1200 mL) was added KHMDS (543 mL, 5.95 equiv) dropwise at −78′C under nitrogen atmosphere. The resulting mixture was stirred for 1h at −78° C. under nitrogen atmosphere. To the above mixture was added 2-(benzenesulfonyl)-3-phenyloxaziridine (136.26 g, 521.453 mmol, 1.3 equiv) in portions over 30 min at −65° C. The resulting mixture was stirred for additional 3h at −65° C. The mixture was allowed to cool down to room temperature. The reaction was quenched by the addition of sat. NH₄Cl (aq.) (1200)mL) at room temperature. The aqueous layer was extracted with DCM (3×500 mL). The resulting mixture was concentrated under reduced pressure. The residue was purified by reversed-phase flash chromatography with the following conditions: column, C18 silica gel; mobile phase, MeCN in Water (10 mmol/L NH₄-1CO₃), 40% to 60% gradient in 10 min; detector. UV 254 nm. This resulted in 2-2 (41 g, 27.57%) as a yellow oil.

### Synthesis of 2-3

A solution of 2-2 (41 g, 130.091 mmol, 1.00 equiv) and NH₂NH₂·H₂O (65.13 g, 1300.910 mmol, 10 equiv) in EtOH (410.00 mL) was stirred overnight at 80° C. The reaction was quenched with NH₄Cl (aq.) (500 mL) at room temperature. The resulting mixture was filtered; the tilter cake was washed with water (3×10) mL). The filtrate was concentrated under reduced pressure. This resulted in 2-3 (29 g, 65.14%) as a white solid.

### Synthesis of 2-4

A solution of 2-3 (29 g, 96.301 mmol, 1.00 equiv) and methyl isothiocyanate (14.08 g, 192.602 mmol, 2.00 equiv) in THE (290 mL) was stirred overnight at room temperature.

The reaction was quenched by the addition of water (400 mL) at room temperature. The resulting mixture was concentrated under reduced pressure. The precipitated solids were collected by filtration and washed with water (3×100 mL). This resulted in 2-4 (32 g, 75.47%) as an off-white solid.

### Synthesis of 2-5

A solution of 2-4 (32 g, 85.504 mmol, 1 equiv) and NaOH (10.26 g, 256.512 mmol, 3 equiv) in H₂O (320 mL) was stirred overnight at room temperature. The mixture was acidified to pH 5 with 2M HCl (aq.). The precipitated solids were collected by filtration and washed with water (3×100 mL). This resulted in 2-5 (30 g, 78.79%) as a yellow solid.

### Synthesis of 2-6

To a stirred solution of 2-5 (30 g, 84.213 mmol, 1 equiv) and NaNO₂ (58.10 g, 842.130 mmol, 10 equiv) in H₂O (300 mL) and EA (50 mL) was added HNO₃ (842 mL, 842.130 mmol, 10.00 equiv, 1 M) dropwise at room temperature. The resulting mixture was stirred for 4 h at room temperature. The reaction was quenched with NaHCO₃ (aq.) (800 mL) at room temperature. The aqueous layer was extracted with EtOAc (3×400 mL). The resulting mixture was concentrated under reduced pressure. The residue was purified by silica gel column chromatography, eluted with PE/EA (5:1) to afford 2-6 (25 g, 82.42%) as a brown solid.

### Synthesis of 2-7

To a stirred solution of 2-6 (25 g, 77.118 mmol, 1.00 equiv) in DCM (250 mL) was added DAST (24.86 g, 154.236 mmol, 2 equiv) dropwise at 0° C. The resulting mixture was stirred for 1h at 0° C. The reaction was quenched with water at room temperature. The aqueous layer was extracted with EtOAc (3×200 mL). The resulting mixture was concentrated under reduced pressure. The residue was purified by silica gel column chromatography, eluted with PE/EA (1:1) to afford 2-7 (16.2 g, 57.96%) as a yellow solid.

### Synthesis of 2-8

To a stirred solution of 2-7 (16.2 g, 49.668 mmol, 1.00 equiv) and NH)·H₂O (200 mL) in MeCN (200 mL) was added Cu₂O (0.71 g, 4.967 mmol, 0.1 equiv) at room temperature. The resulting mixture was stirred overnight at 100° C. The resulting mixture was concentrated under reduced pressure. The residue was purified by reversed-phase flash chromatography with the following conditions: column, C18 silica gel; mobile phase, MeCN in Water (10 mmol/L NHCO₃), 20% to 30% gradient in 10 min; detector, UV 254 nm. This resulted in 2-8 (8.5 g, 59.38%) as a grey solid.

### Synthesis of 2-9

To a stirred solution of 2-8 (8.4 g, 32.026 mmol, 1.00 equiv) and 5-bromo-3-(trifluoromethyl)pyridine-2-carbaldehyde (16.27 g, 64.052 mmol, 2 equiv) in DCE (150 mL) was added NaBH(OAc)₃ (13.58 g, 64.052 mmol, 2 equiv) at room temperature. The resulting mixture was stirred for overnight at room temperature. The reaction was quenched by the addition of NaHCO₃ (aq.) (200 mL) at room temperature. The aqueous layer was extracted with DCM (3×100 mL). The resulting mixture was concentrated under reduced pressure. The residue was purified by silica gel column chromatography, eluted with DCM/MeOH (30:1) to afford 2-9 (10 g, 58.67%) as a yellow solid.

### Synthesis of 2-10

To a stirred solution of 2-9 (9.9 g, 19.788 mmol, 1.00 equiv) and pyridine (9.39 g, 118.728 mmol, 6 equiv) in DCM (100 mL) was added triphosgene (2.35 g, 7.915 mmol, 0.40 equiv) at 0′C. The resulting mixture was stirred for 30 min at room temperature. The reaction was quenched with NaHCO₃ (aq.) (150 mL) at room temperature. The aqueous layer was extracted with DCM (3×100 mL). The residue was purified by trituration with DCM (20 mL). to afford 2-10 (10 g, 91.22%) as a yellow solid.

### Synthesis of 2-11

To a stirred solution of 2-10 (10 g, 19.001 mmol, 1.00 equiv) and Pd(OAc)₂ (0.43 g, 1.900 mmol, 0.1 equiv) in dioxane (200 mL) was added TMEDA (4.42 g, 38.002 mmol, 2 equiv) and bis(adamantan-1-yl) (butyl)phosphane (2.76 g, 3.800 mmol, 0.2 equiv) at room temperature. The resulting mixture was stirred for overnight at 80° C. under carbon monoxide atmosphere. The resulting mixture was concentrated under reduced pressure. The residue was purified by silica gel column chromatography, eluted with DCM/MeOH (20:1) to afford 2-11 (5.8 g, 59.71%) as a yellow solid.

### Synthesis of 2-12

To a stirred solution of 2-11 (400 mg, 0.841 mmol, 1 equiv) and EON (127.71 mg, 1.261 mmol, 1.5 equiv) in DCE (5 mL) was added 1-methylcyclobutan-1-amine hydrochloride (153.48 mg, 1.261 mmol, 1.5 equiv) at room temperature. The resulting mixture was stirred for 1 h at room temperature. To the above mixture was added STAB (356.65 mg, 1.682 mmol, 2 equiv) at room temperature. The resulting mixture was stirred overnight at room temperature. The reaction was quenched with NaHCO₃ (aq.) (30 mL) at room temperature. The aqueous layer was extracted with DCM (3×10 mU. The resulting mixture was concentrated under reduced pressure. The residue was purified by reversed-phase flash chromatography with the following conditions: column, C18 silica gel; mobile phase, MeCN in Water (10 mmol/L NH₄HCO₃), 20% to 30% gradient in 10 min; detector, UV 254 nm. This resulted in 2-12 (200 mg, 42.34%) as a yellow solid.

### Synthesis of Compound 2

The 2-12 (200 mg) was purified by Prep-HPLC with the following conditions (Column: CHIRALPAK 1G, 2*25 cm, 5 μm; Mobile Phase A: Hex (0.5% 2M NH₃-MeOH). Mobile Phase B: EtOH:DCM=1:1--HPLC; Flow rate: 20 mL/min; Gradient: 50% B to 50% B in 16 min; Wave Length: 220/254 nm; RT1 (min): 9.41; RT2 (min): 11.5; the first peak is product) to afford Compound 2 (33.2 mg, 16.79%) as a yellow solid.

LC-MS-Compound 2: (ES, m/z): [M+H]⁺ 545 H-NMR-Compound 2: (400 MHz, DMSO, a ppm): 1.22 (s, 3H), 1.64-1.73 (m, 4H), 1.95-1.97 (m, 2H), 3.31 (s, 3H), 3.44 (s, 2H), 4.84-4.87 (m, 1H), 5.17-5.21 (nm, 2H), 5.33-5.35 (n, 1H), 6.22-6.33 (d, 1H), 7.11-7.13 (m, 2H), 7.31 (s, 1H), 7.41-7.45 (m, 1H), 7.56 (s, 1H), 7.70 (s, 1H), 7.78-7.80 (m, 1H), 8.37 (s, 1H).

### Example 3. Synthesis of Compound 3

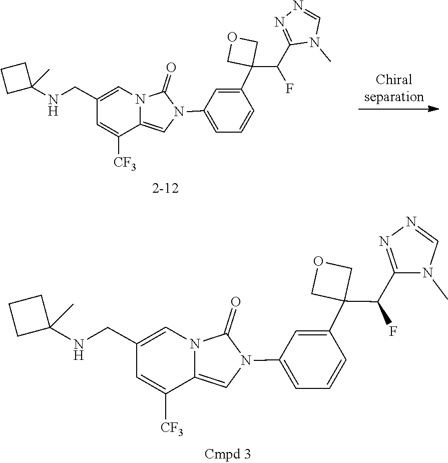 [SMILES: Cn1cnnc1C(F)C1(c2cccc(-n3cc4c(C(F)(F)F)cc(CNC5(C)CCC5)cn4c3=O)c2)COC1 ; Cn1cnnc1[C@@H](F)C1(c2cccc(-n3cc4c(C(F)(F)F)cc(CNC5(C)CCC5)cn4c3=O)c2)COC1]

The 2-12 (200 mg) was purified by Prep-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1--HPLC; Flow rate: 20 mL/min: Gradient: 50% B to 50% B in 16 min: Wave Length: 220/254 nm; RT1 (min): 9.41; RT2 (min): 11.5; the second peak is product) to afford Compound 3 (35.2 mg, 17.62%) as a yellow solid.

LC-MS-Compound 3: (ES, m/z): [M+H]⁺ 545

H-NMR-Compound 3: (400 MHz, DMSO-d6, 5 ppm): 1.23 (s, 3H), 1.65-1.75 (m, 4H), 1.93-1.98 (m, 2H), 3.27-3.31 (m, 3H), 3.45 (s, 1H), 4.84-4.87 (m, 1H), 5.17-5.22 (m, 2H), 5.34-5.35 (m, 1), 6.22-6.33 (d, 1H), 7.11-7.14 (in, 2H), 7.31 (s, 1H), 7.41-7.43 (m, 11H), 7.56 (s, 1H), 7.57 (s, 1H), 7.78-7.81 (m, 1H), 8.37 (s, 1H).

### Example 4. Synthesis of Compound 4

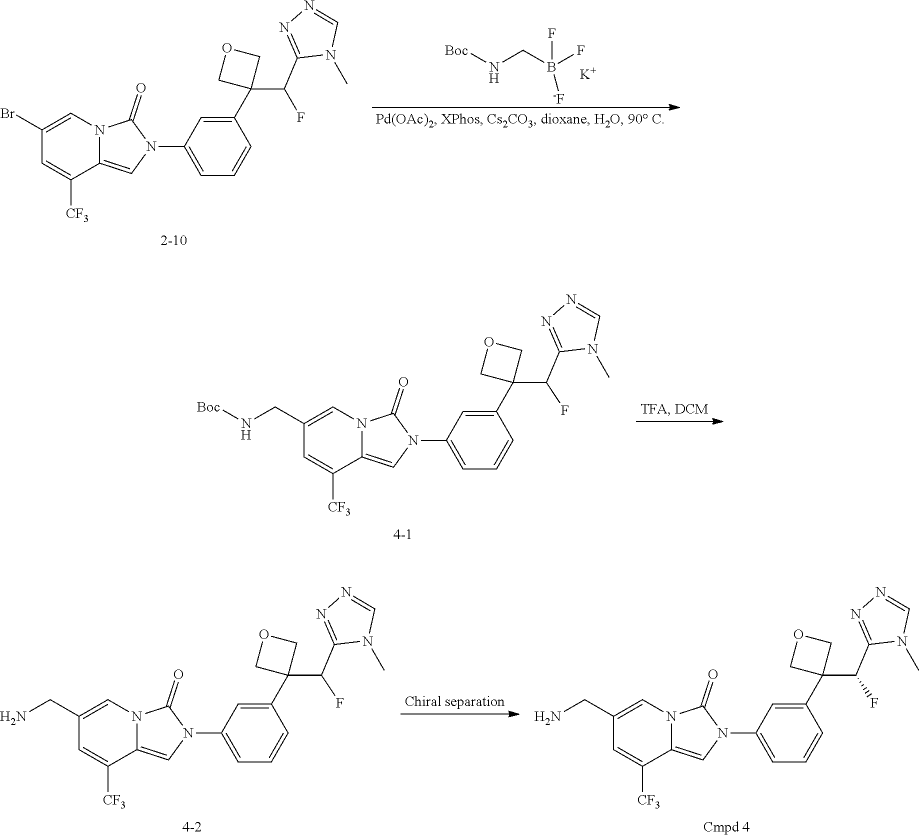

### Synthesis of 4-1

To a stirred solution of 2-10 (500 mg, 0.950 mmol, 1 equiv) and Pd(OAc)₂ (21.33 mg, 0.095 mmol, 0.1 equiv) in dioxane (5 mL) and 110 (1.5 mL) were added XPhos (90.58 mg, 0.190 mmol, 0.2 equiv) and Cs₂CO₃ (928.63 mg, 2.850 mmol, 3 equiv) and potassium tert-butyl N-[(trifluoroboranuidyl)methyl]carbamate (337.84 mg, 1.425 mmol, 1.5 equiv) at room temperature under nitrogen atmosphere. The resulting mixture was stirred for 3 h at 80° C. under nitrogen atmosphere. The reaction was quenched with water at room temperature. The aqueous layer was extracted with DCM (3×10 mL). The residue was purified by Prep-TLC (DCM/MeOH 30:1) to afford 4-1 (240 mg, 37.24%) as a brown solid. The resulting mixture was concentrated under reduced pressure.

### Synthesis of 4-2

A solution of 4-1 (240 mg, 0.416 mmol, 1 equiv) and TFA (0.8 mL) in DCM (4 mL) was stirred for 2 h at room temperature. The resulting mixture was concentrated under vacuum. The residue was purified by reversed-phase flash chromatography with the following conditions: column, C18 silica gel; mobile phase, MeCN in Water (0.1% FA), 20% to 30% gradient in 10 min; detector, UV 254 nm. This resulted in 4-2 (100 mg, 49.41%) as a grey solid.

### Synthesis of Compound 4

The crude product 4-2 (200 mg) was purified by Prep-HPLC with the following conditions (Column: CHIRALPAK IC, 2*25 cm, 5 μm; Mobile Phase A: Hex(0.5% 2M NH₃-MeOH), Mobile Phase B: MEOH:DCM=1:1--HPLC; Flow rate: 20 mL/min: Gradient: 90% B to 90% B in 16 min: Wave Length: 220/254 nm; RT1 (min): 10.16: RT2 (min): 13.03; the first peak is product) to afford Compound 4 (13.2 mg, 6.71%) as a yellow solid.

LC-MS-Compound 4: (ES, m/z): [M+H]⁺ 476

H-NMR-Compound 4: (400 MHz, CD₃OD, δ ppm): 3.19 (s, 1H), 3.68 (s, 1H), 5.01-5.03 (m, H), 5.22-5.24 (m, 1H), 5.30-5.31 (m, 1H), 5.45-5.47 (m, 1W), 6.24-6.35 (d, 1H), 7.08-7.09 (m, 2H), 7.10-7.13 (m, 1H), 7.44-7.49 (m, 2H), 7.62-7.67 (m, 2H), 8.28 (s, 1H).

### Example 5. Synthesis of Compound 5

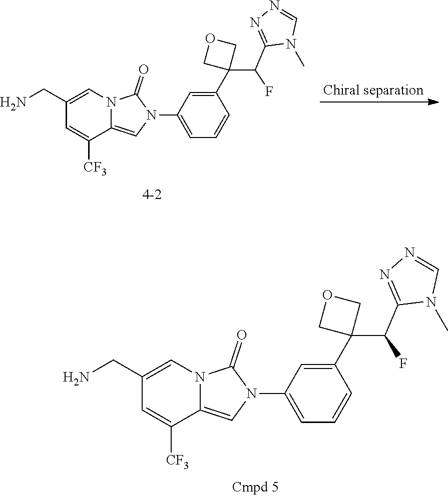 [SMILES: Cn1cnnc1C(F)C1(c2cccc(-n3cc4c(C(F)(F)F)cc(CN)cn4c3=O)c2)COC1 ; Cn1cnnc1[C@@H](F)C1(c2cccc(-n3cc4c(C(F)(F)F)cc(CN)cn4c3=O)c2)COC1]

The 4-2 (200 mg) was purified by Prep-HPLC with the following conditions (Column: CHIRALPAK IC, 2*25 cm, 5 μm; Mobile Phase A: Hex (0.5% 2M NH₃-MeOH), Mobile Phase B: MEOH:DCM=1:1--HPLC; Flow rate: 20 mL/min; Gradient: 90% B to 90% B in 16 min; Wave Length: 220/254 nm; RT1 (min): 10.16; RT2 (min): 13.03; the second peak is product) to afford Compound 5 (12.7 mg, 6.41%) as a yellow solid.

LC-MS-Compound 5: (ES, m/z): [M+H]⁺ 476

H-NMR-Compound 5: (400 MHz, CD₃OD, δ ppm): 3.20 (m, 3H), 3.62 (m, 2H), 5.00-5.03 (m, H), 5.22-5.23 (m, 1H), 5.29-5.31 (m, 1H), 5.45-5.47 (m, 1W), 6.24-6.33 (d, 1H), 7.08-7.13 (m, 311), 7.63-7.67 (m, 2H), 8.28 (s, 1H).

### Example 6. Synthesis of Compound 6

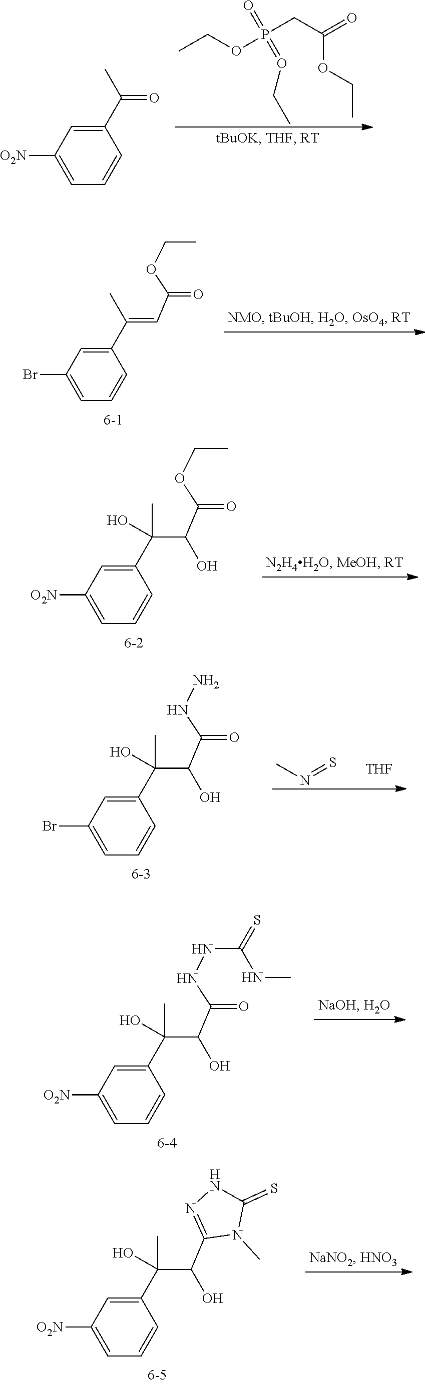

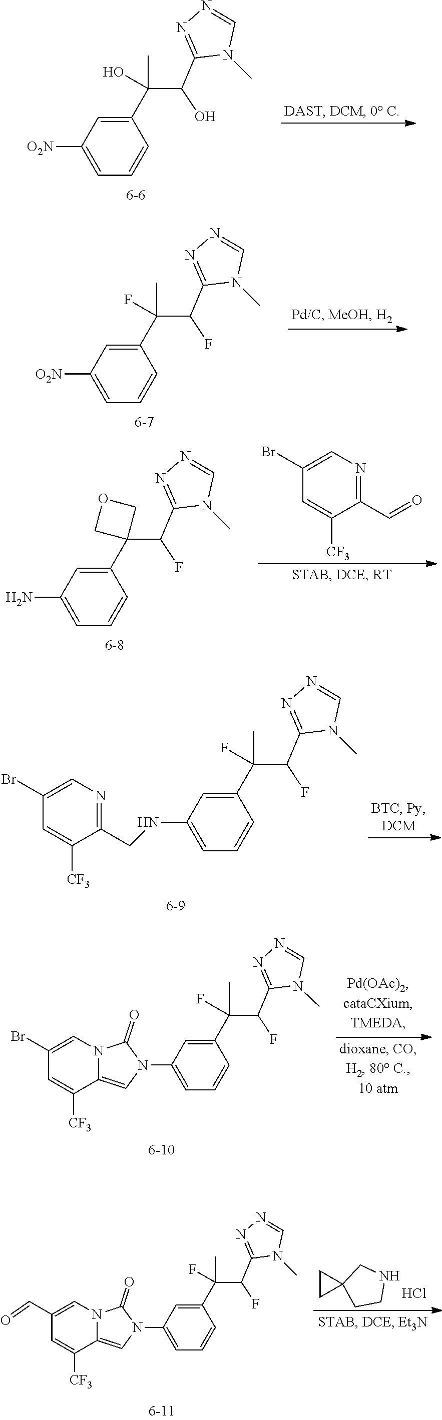

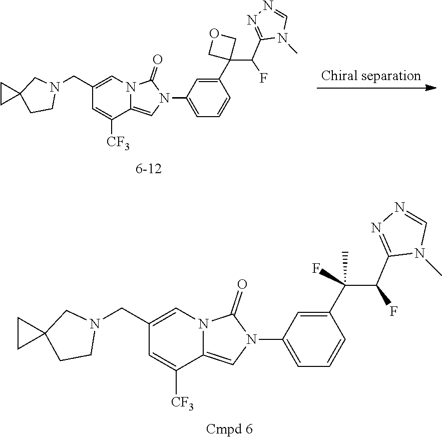 [SMILES: Cn1cnnc1C(F)C1(c2cccc(-n3cc4c(C(F)(F)F)cc(CN5CCC6(CC6)C5)cn4c3=O)c2)COC1 ; Cn1cnnc1[C@@H](F)[C@](C)(F)c1cccc(-n2cc3c(C(F)(F)F)cc(CN4CCC5(CC5)C4)cn3c2=O)c1]

### Synthesis of 6-2

To a stirred solution of triethyl phosphonoacetate (81.45 g, 363.311 mmol, 1.2 equiv) in THF (800 mL) were added t-BuOK (5096 g, 454.139 mmol, 1.5 equiv) at 0° C. under nitrogen atmosphere. The resulting mixture was stirred for 1h at 0° C.₂. To the above mixture was added 3-nitroacetophenone (50 g, 302.759 mmol, 1 equiv) at 0° C. The resulting mixture was stirred for additional overnight at room temperature. The reaction was quenched with NH₂Cl (aq.) (1500 mL) at room temperature. The resulting mixture was extracted with CH₂Cl₂ (3×1000 mL). The resulting mixture was, concentrated under reduced pressure. The residue was purified by silica gel column chromatography, eluted with PE/1EA (5:1) to afford 6-1 (60 g, 75.82%) as a yellow oil.

### Synthesis of 6-2

To a stirred solution of 6-1 (30 g, 127.530 mmol, 1 equiv) and NMO (22.41 g, 191.295 mmol, 1.5 equiv) in t-BuOH (300 mL) were added 1-120 (300 mL) and OsO₄ (3.24 g, 12.753 mmol, 0.1 equiv) at room temperature. The resulting mixture was stirred overnight at room temperature. The reaction was quenched with HCl (aq.) (1000 mL) at room temperature. The resulting mixture was extracted with EtOAc (3×1000 mL). The resulting mixture was concentrated under reduced pressure. The residue was purified by silica gel column chromatography, eluted with PE/EA (10:1) to afford 6-2 (25 g, 65.53%) as a yellow oil.

### Synthesis of 6-3

To a stirred solution of 6-2 (25 g, 92.849 mmol, 1 equiv) in MeOH (3M) mL) were added hydrazine hydrate (46.48 g, 928.490 mmol, 10 equiv) at 0° C. The resulting mixture was stirred overnight at room temperature. The reaction was quenched with water (600 mL) at room temperature. The resulting mixture was extracted with CH₂Cl₂ (3×1000 mL). The resulting mixture was concentrated under reduced pressure. This resulted in 6-3 (18 g, 66.84%) as an orange oil.

### Synthesis of 6-4

To a stirred solution of 6-3 (18 g, 70.525 mmol, 1 equiv) in tetrahydrofuran (300 mL) were added methyl isothiocyanate (7.73 g, 105.788 mmol, 1.5 equiv) at room temperature.

The resulting mixture was stirred overnight at room temperature. The resulting mixture was concentrated under reduced pressure. The residue was purified by silica gel column chromatography, eluted with CH₂Cl₂/MeOH (20:1) to afford 6-4 (15 g, 58.95%) as an orange oil.

### Synthesis of 6-5

To a stirred solution of 6-4 (15 g, 45.684 mmol, 1 equiv) in H₂O (200 mL) was added NaOH (3.65 g, 91.368 mmol, 2 equiv) at room temperature. The resulting mixture was stirred for 2 h at room temperature. The mixture was neutralized to pH 7 with HIC (aq.). The precipitated solids were collected by filtration and washed with water (2×50 ml). This resulted in 6.5 (15 g, 84.64%) as an orange solid.

### Synthesis of 6-6

To a stirred solution of 6-5 (15 g, 48.336 mmol, 1 equiv) and NaNO₂ (33.35 g, 483.360 mmol, 10 equiv) in H₂O (200 mL) were added HNO₃ (6.09 g, 96.672 mmol, 2 equiv) dropwise at 0° C. The resulting mixture was stirred for overnight at room temperature. The mixture was neutralized to pH 7 with NaHCO₃ (aq.). The resulting mixture was extracted with CH₂Cl₂ (3×500 mL). The resulting mixture was concentrated under reduced pressure. The residue was purified by silica gel column chromatography, eluted with CH₂Cl₂/MeOH (20:1) to afford 6-6 (5.5 g, 38.85%) as an orange oil.

### Synthesis of 6-7

To a stirred solution of 6-6 (5.5 g, 19.765 mmol, 1 equiv) in DCM (100 mL) was added DAST (15.93 g, 98.825 mmol, 5 equiv) dropwise at 0° C. The resulting mixture was stirred for 2 h at 0° C. The reaction was quenched with NaHCO₃ (aq.) (3M) ml) at 0° C. The residue was purified by silica gel column chromatography, eluted with CH₂Cl₂/MeOH (50:1) to afford 6-7 (3.5 g, 40.8%) as an orange oil.

### Synthesis of 6-8

To a stirred solution of 6-7 (3.5 g, 12.400 mmol, 1 equiv) in MeOH (50 mL) were added Pd/C (0.40 g, 3.720 mmol, 0.3 equiv) at room temperature under nitrogen atmosphere. The resulting mixture was stirred overnight at room temperature under hydrogen atmosphere. The resulting mixture was filtered, the tilter cake was washed with MeOH (2×5 ml). The resulting mixture was concentrated under reduced pressure. This resulted in 6-8 (1.7 g, 52.17%) as an orange oil.

### Synthesis of 6-9

To a stirred solution of 6-8 (1.7 g, 6.739 mmol, 1 equiv) and 5-bromo-3-(trifluoromethyl)pyridine-2-carbaldehyde (2.05 g, 8.087 mmol, 1.2 equiv) in DCE (30 mL) were added STAB (2.86 g, 13.478 mmol, 2 equiv) and HOAc (0.40 g, 6.739 mmol, 1 equiv) at room temperature. The resulting mixture was stirred overnight at room temperature. The reaction was quenched with NHCO₃ (aq.) (100 mL) at room temperature. The resulting mixture was extracted with CH₂Cl₂ (3×50 mL). The resulting mixture was concentrated under reduced pressure. The residue was purified by silica gel column chromatography, eluted with CH₂Cl₂/MeOH (50:1) to afford 6-9 (2.7 g, 79.27%) as an orange oil.

### Synthesis of 6-10

To a stirred solution of 6-9 (2.7 g, 5.507 mmol, 1 equiv) and pyridine (2.61 g, 33.042 mmol, 6 equiv) in DCM (50 mL) were added Triphosgene (0.57 g, 1.927 mmol, 0.35 equiv) at 0° C. The resulting mixture was stirred for 20 min at room temperature. The reaction was quenched by the addition of NaHCO₃ (aq.) (100 mL) at room temperature. The residue was purified by silica gel column chromatography, eluted with CH₂C₂/MeOH (20:1) to afford 6-10 (2.2 g, 73.51%) as a yellow solid.

### Synthesis of 6-11

To a stirred solution of 6-10 (2.2 g, 4.261 mmol, 1 equiv) and TMEDA (0.99 g, 8.522 mmol, 2 equiv) in dioxane (50 mL) were added bis(adamantan-1-yl) (butyl)phosphane (0.31 g, 0.852 mmol, 0.2 equiv) and Pd(AcO)₂ (0.10 g, 0.426 mmol, 0.1 equiv) at room temperature. The resulting mixture was stirred overnight at 80° C. under H₂:CO (1:1) atmosphere. The resulting mixture was concentrated under reduced pressure. The residue was purified by silica gel column chromatography, eluted with CH₂C₂/MeOH (50:1) to afford 6-11 (1.7 g, 77.15%) as a yellow solid.

### Synthesis of 6-12

To a stirred solution of 5-azaspiro[2.4]heptane hydrochloride (0.59 g, 4.384 mmol, 1.2 equiv) and 6-11 (1.7 g, 3.653 mmol, 1.00 equiv) in DCE (30 mL) were added TEA (0.55 g, 5.479 mmol, 1.5 equiv) at room temperature. The resulting mixture was stirred for 1h at room temperature. To the above mixture was added STAB (1.55 g, 7.306 mmol, 2 equiv) at room temperature. The resulting mixture was stirred for additional 2h at room temperature.

The reaction was quenched with NH₄Cl (aq.) (60 mL at room temperature. The resulting mixture was extracted with CH₂Cl₂ (3×100 mL). The resulting mixture was concentrated under reduced pressure. The residue was purified by reversed-phase flash chromatography with the following conditions: column, C18 silica gel; mobile phase. MeCN in Water (10 mmol/L NH₄HCO₃), 10% to 50% gradient in 10 mint; detector, UV 254 nm. This resulted in 6-12 (1.2 g, 57.70%) as a yellow solid.

### Synthesis of Compound 6

The 6-12 (400 mg) was purified by Prep-Chiral-HPLC with the following conditions (Column: CHIRALPAK 1H, 2*25 cm, 5 μm: Mobile Phase A: Hex (0.5% 2M NH₃-MeOH), Mobile Phase B: EtOH:DCM=1:1--HPLC; Flow rate: 20 mL/min; Gradient: 25% B to 25% B in 14 min; Wave Length: 220/254 nm; RT1 (min: 6.29; RT2 (min): 7.48; the first peak is product) to afford the crude product. This resulted in Compound 6 (37.5 mg, 9.37%) as a yellow solid.

LC-MS-Compound 6: (ES, m/z): [M+H]⁺ 547

H-NMR-Compound 6: (400 MHz, DMSO-d6, ppm): δ 4.47-4.53 (m, 4H), δ 1.73-1.76 (m. 2H), δ 1.92-1.99 (m, 3H), δ 2.45-2.49 (in, 2H), δ 2.68 (s, 2H), δ 3.34-3.40 (d, 2H), δ 3.56 (s, 3H), δ 6.28-6.45 (m, 1H), δ 7.05 (s, 1H), δ 7.25-7.27 (d, 1H), δ7.34 (s, 1H), δ 7.46-7.50 (m, 1H), δ7.69 (s, 1H), δ7.76 (s, 1H), δ 7.58-7.87 (m, 1H), δ 8.44 (s, 1H).

### Example 7. Synthesis of Compound 7

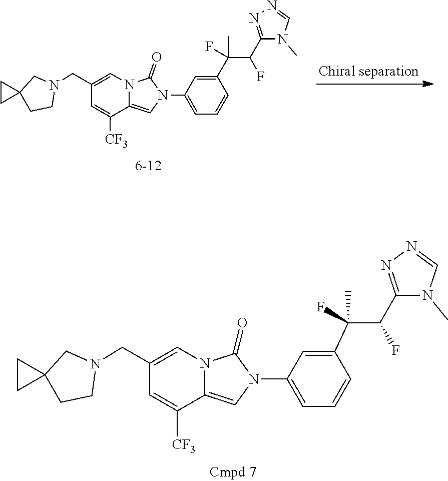 [SMILES: Cn1cnnc1C(F)C(C)(F)c1cccc(-n2cc3c(C(F)(F)F)cc(CN4CCC5(CC5)C4)cn3c2=O)c1 ; Cn1cnnc1[C@H](F)[C@](C)(F)c1cccc(-n2cc3c(C(F)(F)F)cc(CN4CCC5(CC5)C4)cn3c2=O)c1]

The 6-12 (400 mg) was purified by Prep-Chiral-HPLC with the following conditions (Column: CHIRALPAK 1H, 2*25 cm, 5 μm; Mobile Phase A: Hex(0.5% 2M NH₃-MeOH), Mobile Phase B: EtOH:DCM=1:1--HPLC; Flow rate: 20 mL/min: Gradient: 25% B to 25% B in 14 min: Wave Length: 220/254 nm; RT1 (min): 6.29; RT2 (min): 7.48; the second peak is product) to afford the crude product. This resulted in Compound 7 (45.5 mg, 11.09%) as a yellow solid.

LC-MS-Compound 7: (ES, m/z): [M+H]⁺ 547

H-NMR-Compound 7: (400 MHz, DMSO-d6, ppm): δ 4.47-4.53 (m, 4H), δ 1.73-1.76 (m, 2H), δ 1.92-1.99 (m, 3H), δ 2.45-2.49 (m, 2H), δ 2.68 is, 2H), δ 3.34-3.40 (d, 2H), δ 3.56 (s, 3H), δ 6.28-6.45 (m, 1H), δ 7.05 (s, 1f), δ 7.25-7.27 (d, 11H), δ 7.34 (s, 1H), δ 7.46-7.50 (m, 1H), δ 7.69 (s, 1H), δ 7.76 (s, 1H), δ 7.58-7.87 (m, 1H), δ 8.44 (s, 1H).

### Example 8. Synthesis of Compound 8

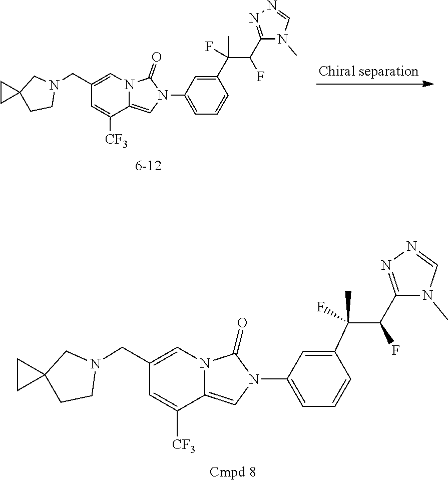 [SMILES: Cn1cnnc1C(F)C(C)(F)c1cccc(-n2cc3c(C(F)(F)F)cc(CN4CCC5(CC5)C4)cn3c2=O)c1 ; Cn1cnnc1[C@@H](F)[C@@](C)(F)c1cccc(-n2cc3c(C(F)(F)F)cc(CN4CCC5(CC5)C4)cn3c2=O)c1]

The 6-12 (400 mg) was purified by Prep-Chiral-HPLC with the following conditions (Column: CHIRALPAK 1H, 2*25 cm, 5 μm: Mobile Phase A: Hex(0.5% 2M NH₃-MeOH), Mobile Phase B: EtOH:DCM=1:1--HPLC; Flow rate: 20 mL/min; Gradient: 25% B to 25% B in 14 min; Wave Length: 220/254 nm; RT1 (min): 6.29; RT2 (min: 7.48; the third peak is product) to afford the crude product. This resulted in Compound 8 (47.8 mg, 11.93%) as a yellow solid.

LC-MS-Compound 8: (ES, m/z): [M+H]⁺ 547

H-NMR-Compound 8: (4 (00 MHz, DMSO-d6, ppm): δ 4.47-4.53 (m, 4H), δ 1.73-1.76 (m, 2H), δ 1.92-1.99 (m, 3H), δ 2.45-2.49 (m, 2H), δ 2.68 (s, 2H), δ 3.34-3.40 (d, 2H), δ 3.56 (s, 3H), δ 6.28-6.45 (m, 1H), δ 7.05 (s, 1H), δ 7.25-7.27 (d, 1H), δ 7.34 (s, 1H), a 7.46-7.50 (m, 1H), δ 7.69 (s, 1H), δ 7.76 (s, 1H), δ 7.58-7.87 (m, 11H), δ 8.44 (s, 1H).

### Example 9. Synthesis of Compound 9

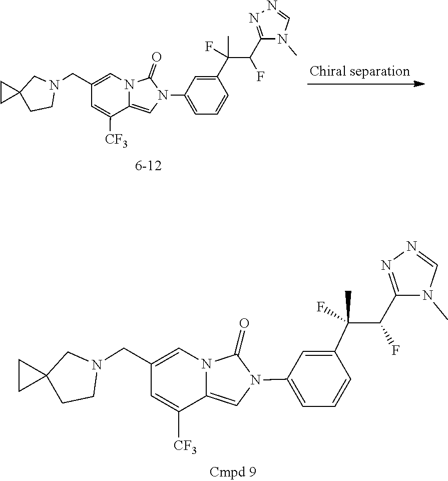 [SMILES: Cn1cnnc1C(F)C(C)(F)c1cccc(-n2cc3c(C(F)(F)F)cc(CN4CCC5(CC5)C4)cn3c2=O)c1 ; Cn1cnnc1[C@H](F)[C@@](C)(F)c1cccc(-n2cc3c(C(F)(F)F)cc(CN4CCC5(CC5)C4)cn3c2=O)c1]

The 6-12 (400 mg) was purified by Prep-Chiral-HPLC with the following conditions (Column: CHIRALPAK IH, 2*25 cm, 5 μm; Mobile Phase A: Hex (0.5% 2M NH₃-MeOH), Mobile Phase B: EtOH:DCM=1:1--HPLC; Flow rate: 20 mL/min: Gradient: 25% B to 25% B in 14 min; Wave Length: 220/254 nm; RT1 (min): 6.29; RT2 (min): 7.48; the fourth peak is product) to afford the crude product. This resulted in Compound 9 (38.7 mg, 9.37%) as a yellow solid.

LC-MS-Compound 9: (ES, m/z): [M+H]⁺ 547

H-NMR-Compound 9: (400 MHz, DMSO-d6, ppm): δ 4.47-4.53 (m, 4H), 1.73-1.76 (m, 2H), δ 1.92-1.99 (m, 3H), δ 2.45-2.49 (m, 2H), δ 2.68 (s, 2H), δ 3.34-3.40 (d, 2H), δ 3.56 (s, 3H), δ 6.28-6.45 (m, 1H), δ 7.05 (s, 1H), δ 7.25-7.27 (d, 1H), δ 7.34 (s, 1H), δ 7.46-7.50 (m, 1H), δ 7.69 (s, 1H), δ 7.76 (s, 1H), δ 7.58-7.87 (m, 1H), δ 8.44 (s, 1H).

### Example 10. Synthesis of Compound 10

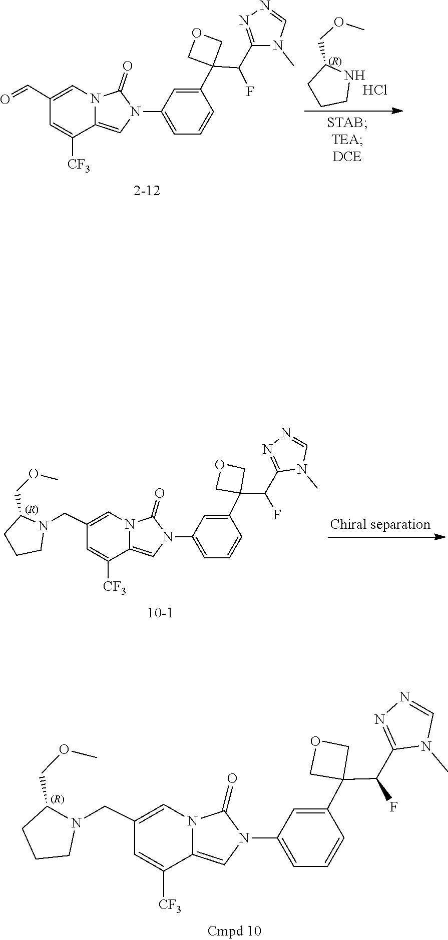

### Synthesis of 10-1

To a stirred solution of 2-12 (400 mg, 0.841 mmol, 1 equiv) and (2R)-2-(methoxymethyl)pyrrolidine (145.36 mg, 1.261 mmol, 1.5 equiv) in DCE (10 ml) was added TEA (170.29 mg, 1.682 mmol, 2 equiv) at room temperature. The resulting mixture was stirred for 2 h at room temperature. To the above mixture was added STAB (356.65 mg, 1.682 mmol, 2 equiv) at room temperature. The resulting mixture was stirred overnight at room temperature. The reaction was quenched with NaHCO₃ (aq.) at room temperature. The aqueous layer was extracted with DCM (3×20 mL). The resulting mixture was concentrated under vacuum. The residue was purified by Prep-TLC (DCM/MeOH 13:1) to afford 10-1 (140 mg, 27.51%) as a yellow solid.

### Synthesis of Compound 10

The 10-1 (150 mg) was purified by Prep-HPLC with the following conditions (Column: CHIRALPAK IC. 2*25 cm, 5 Ium; Mobile Phase A: Hex(0.5% 2M NH₃-MeOH), Mobile Phase B: ETOH:DCM=1:1-HPLC; Flow rate: 20 mL/min; Gradient: 60% B to 60% B in 19 min; Wave Length: 220/254 nm; RT1 (min): 12.28; RT2 (min): 15.28; the second peak is product) to afford Compound 10 (45.4 mg, 31.73%) as a yellow solid.

LC-MS-Compound 10: (ES, m/z): [M+H]⁺ 575

H-NMR-Compound 10: (400 MHz, DMSO, 6 ppm): 1.42-1.51 (m, 1H), 1.63-1.74 (m, 2H), 1.85-1.90 (m, 1H), 2.27-2.24 (m, 1H), 2.71 (s, 1H), 2.86 (s, 1H), 3.22-3.25 (m, 4H), 3.31-3.37 (m, 3H), 3.38 (s, 1H), 3.39-3.41 (m, 1 H), 3.78-3.82 (s, 1H), 4.8-4-4.87 (s, 1H), 5.18-5.20 n, 2H), 5.34-5.35 (m, 2H), 6.22-6.33 (d, 2H), 7.01 (s, 1H), 7.12-7.14 (m, 11-H), 7.34 (s, 1H), 7.41-7.45 (in, 1H), 7.56 (s, 1H), 7.68 (s, 1H), 7.78-1.80 (m, 1H), 8.38 (s, 1H).

### Example 11. Synthesis of Compound 11

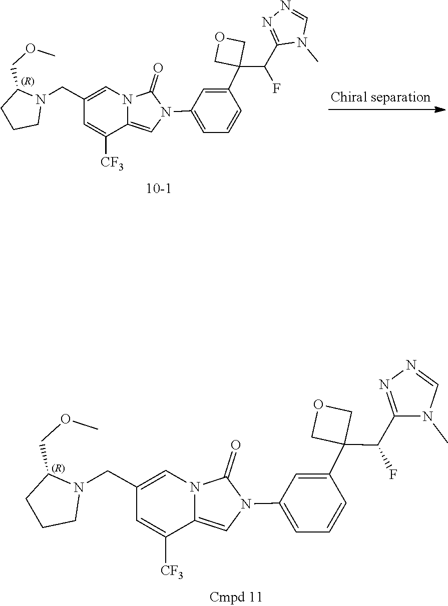

The 10-1 (150 mg) was purified by Prep-HPLC with the following conditions (Column: CHIRALPAK IC, 2*25 cm, 5 μm: Mobile Phase A: Hex (0.5% 2M NHs-MeOH), Mobile Phase B: ETOH:DCM=1:1--HPLC; Flow rate: 20 mL/min; Gradient: 60% B to 60% B in 19 min; Wave Length: 220/254 nm; RT1 (min): 12.28; RT2 (min): 15.28, the first peak is product) to afford Compound 11 (45.2 mg, 31.69%) as a yellow solid.

LC-MS-Compound 11: (ES, m/z): [M+]⁺ 575

H-NMR-Compound 11: (400 MHz, DMSO, 6 ppm): 1.49-1.52 (m, 11H), 1.62-1.66 (m, 2H), 1.85-1.90 (m, 1H), 2.22-2.24 (m, 1H), 2.69-2.72 (s, 1H), 2.86 (s, 1H), 3.21-3.23 (m, 1H), 3.25-3.27 (m, 3H), 3.38-3.39 (m, 3H), 3.39-3.40 (m, 1H), 3.40 (s, 1H), 3.78-3.81 (m, 1H), 4.81-4.87 (m, 1H), 5.18-5.22 (m, 2H), 5.34-5.35 (in, 1H), 6.22-0.33 (d, 21H), 7.00 (s, 11H), 7.12-7.14 (m, 1H), 7.34 (s, 1H), 7.41-7.45 (m, H), 7.56 (s, 1H), 7.68 (s, 1H), 7.78-1.80 (m, 1H), 8.38 (s, 1H).

### Example 12. Synthesis of Compound 12

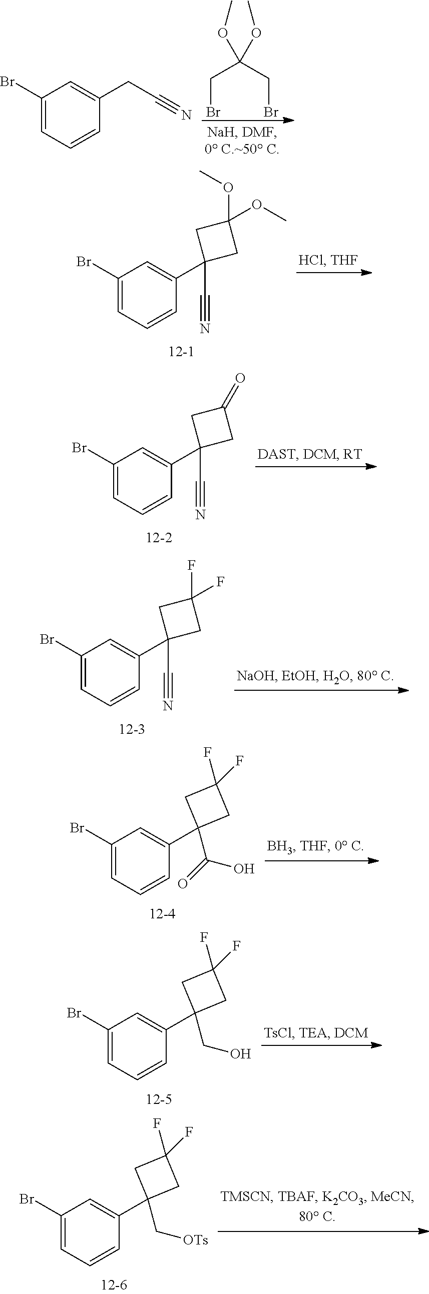

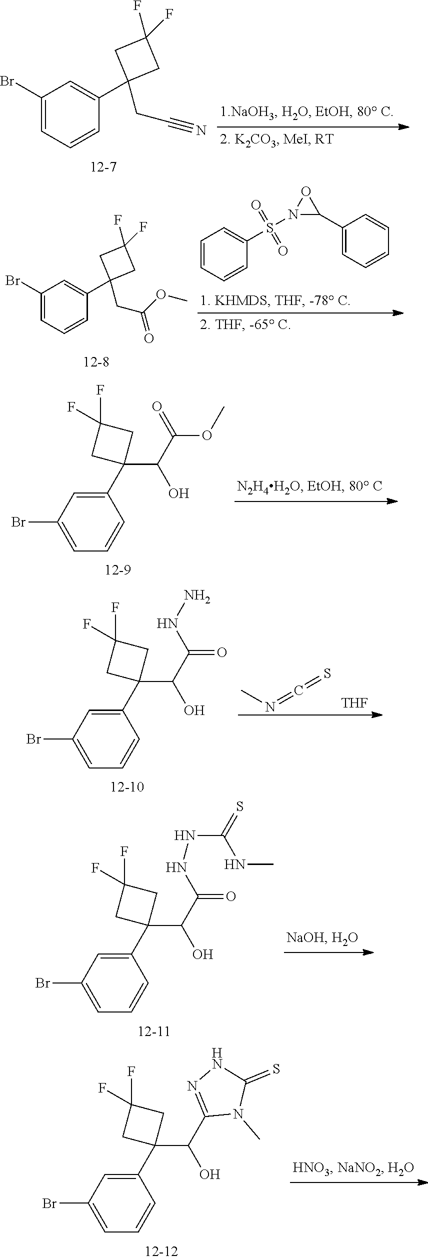

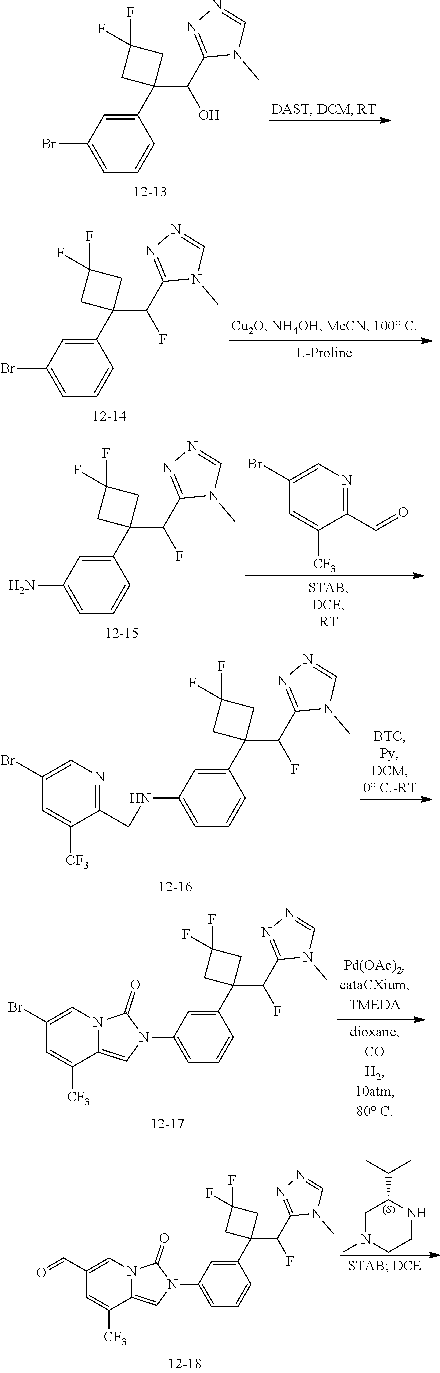

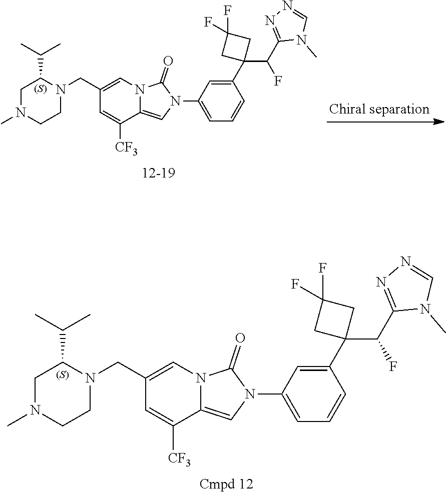

### Synthesis of 12-1

A solution of Na-1 (7.34 g, 306.050 mmol, 2 equiv) in DMF (300 mL) was treated with 2-(3-bromophenyl)acetonitrile (30 g, 153.025 mmol, 1 equiv) for 20 min at 0° C. under nitrogen atmosphere and followed by 1,3-dibromo-2,2-dimethoxypropane (34.07 g, 130.071 mmol, 0.85 equiv) in portions at room temperature. The resulting mixture was stirred overnight at 60′C under nitrogen atmosphere. The reaction was quenched with NH₄C₄ (aq.) at room temperature. The resulting mixture was extracted with EtOAc (3×800 mL). The combined organic layers were washed with water (5×300 mL), dried over anhydrous Na₂SO₄. After filtration, the filtrate was concentrated under reduced pressure. The residue was purified by silica gel column chromatography, eluted with PE/EA (90:1) to afford 12-1 (25.4 g, 56.05%) as a yellow oil.

### Synthesis of 12-2

To a stirred solution of 12-1 (24.4 g, 82.387 mmol, 1 equiv) and HCl (100 mL, 4M) in H₂O (200 mL) was added THF (100 mL) in portions at room temperature under air atmosphere. The resulting mixture was stirred overnight at 80° C. The resulting mixture was concentrated under reduced pressure. The mixture was neutralized to pH 7 with saturated NaHCO₃ (aq.). The aqueous layer was extracted with DCM (3×70 mL).

The resulting mixture was concentrated under reduced pressure. The residue was purified by reversed-phase flash chromatography with the following conditions: column, C18 silica gel: mobile phase, MeCN in Water (10 mmol/L NH₄HCOs), 20% to 70% gradient in 25 min; detector, UV 254 nm. This resulted in 12-2 (18.6 g, 90.27%) as a yellow oil.

### Synthesis of 12-3

To a stirred solution of 12-2 (18.6 g, 74.372 mmol, 1 equiv) in DCM (200 mL) was added DAST (59.94 g, 371.860 mmol, 5 equiv) dropwise at 80° C. The resulting mixture was stirred for overnight at room temperature. The reaction was quenched with NaHCO₃ (aq.) (500 mL) at 0° C. The aqueous layer was extracted with DCM (3×200 mL). The resulting mixture was concentrated under vacuum. The residue was purified by reversed-phase flash chromatography with the following conditions: column, C18 silica gel; mobile phase. MeCN in Water (10 mmol/L NH₄HCO₃), 20% to 70% gradient in 35 min; detector, UV 254 nm. This resulted in 12-3 (13.2 g, 61.97%) as a white solid.

### Synthesis of 12-4

To a stirred solution of NaOH (8.82 g, 220.515 mmol, 5 equiv). EtOH (20 mL) in H₂O (100 mL) was added 12-3 (12 g, 44.103 mmol, 1 equiv) at room temperature. The resulting mixture was stirred overnight at 80° C. The resulting mixture was concentrated under vacuum. The mixture was acidified to pH 1 with HCl(1M). The precipitated solids were collected by filtration and washed with water (2×50 mL). This resulted in 12-4 (11 g, 81.40%) as a white solid.

### Synthesis of 12-5

A solution of 12-5 (II g, 37.789 mmol, 1 equiv) and oxolane borane (9.74 g, 113.367 mmol, 3 equiv) in THF (120 mL) was stirred for 3 h at 0° C. The reaction was quenched with NH₄Cl(aq.) (150 mL) at 0° C. The aqueous layer was extracted with DCM (3×100 mL). The resulting mixture was concentrated under vacuum. The residue was purified by reversed-phase flash chromatography with the following conditions: column, C18 silica gel; mobile phase, MeCN in Water (0.1% FA), 20% to 30% gradient in 10 min; detector, UV 254 nm.

This resulted in 12-5 (11 g, 94.54%) as a grey oil.

### Synthesis of 12-6

To a stirred solution of 12-4 (11 g, 39.696 mmol, 1 equiv) and TsCl (15.14 g, 79.392 mmol, 2 equiv) in DCM (110 mL) was added TEA (12.05 g, 119.088 mmol, 3 equiv) dropwise at 0° C. under nitrogen atmosphere. The resulting mixture was stirred for 2 h at room temperature under nitrogen atmosphere. The reaction was quenched with water (200 mL) at room temperature. The aqueous layer was extracted with DCM (3×100 mL. The resulting mixture was concentrated under vacuum. The residue was purified by reversed-phase flash chromatography with the following conditions: column, C18 silica gel; mobile phase, MeCN in Water (0.1% FA), 25% to 70% gradient in 40 min; detector, UV 254 nm.

This resulted in 12-5 (9 g, 46.79%) as a yellow solid.

### Synthesis of 12-7

To a stirred solution of 12-6 (5.5 g, 12.752 mmol, 1 equiv) and TMSCN (1.90 g, 19.128 mmol, 1.5 equiv) in TBAF (6.67 g, 25.504 mmol, 2 equiv) was added K₂CO₃ (5.29 g, 38.256 mmol, 3 equiv) at room temperature. The resulting mixture was stirred overnight at 80° C. The reaction was quenched with sat. Na₂S₂O₃ (aq.) at room temperature. The aqueous layer was extracted with DCM (3×100 mL). The residue was purified by silica gel column chromatography, eluted with PE/EA (20:1) to afford 12-7 (3 g, 74.00%) as a brown solid.

### Synthesis of 12-8

A solution of 12-7 (3 g, 10.485 mmol, 1 equiv) and NaOH (4.19 g, 104.850 mmol, 10 equiv) in H₂O (30 mL) and EtOH (30 mL) was stirred overnight at 80° C. The mixture was acidified to pH 5 with HCl (aq.). The precipitated solids were collected by filtration and washed with water (3×20 mL). This resulted in [1-43-bromophenyl)-3,3-difluorocyclobutyl]acetic acid (2.9 g, 81.58%) as a grey solid. To a stirred solution of [1-(3-bromophenyl)-3,3-difluorocyclobutyl]acetic acid (2.7 g, 8.849 mmol, 1 equiv) and K₂CO₃ (3.67 g, 26.547 mmol, 3 equiv) in MeCN (40 mL) was added Mel (5.02 g, 35.396 mmol, 4 equiv) at room temperature. The resulting mixture was stirred overnight at 50° C. The reaction was quenched by the addition of water (100 mL) at room temperature. The aqueous layer was extracted with DCM (3×30 ml). The resulting mixture was concentrated under reduced pressure. This resulted in 12-8 (3 g, 95.60%) as a grey oil.

### Synthesis of 12-9

To a stirred solution of 12-8 (2.9 g, 9.087 mmol, 1 equiv) in THF (40 mL) was added KHMDS (3.63 g, 18.174 mmol, 2 equiv) at −78° C. under nitrogen atmosphere. The resulting mixture was stirred for 1h at −78° C. To the above mixture was added 2-(benzenesulfonyl)-3-phenyloxaziridine (3.56 g, 13.624 mmol, 1.50 equiv) dropwise at −78° C. The resulting mixture was stirred for additional 4h at −60° C. The reaction was quenched with NH₄Cl(aq.) at room temperature. The aqueous layer was extracted with EtOAc (3×50 mL). The resulting mixture was concentrated under vacuum. The residue was purified by reversed-phase flash chromatography with the following conditions: column, C18 silica gel; mobile phase. MeCN in Water (0.1% FA), 50% to 60% gradient in 10 min; detector, UV 254 nm. This resulted in 12-9 (2 g, 59.11%) as a yellow oil.

### Synthesis of 12-10

A solution of 12.9 (2 g, 5.968 mmol, 1 equiv) and hydrazine hydrate (2.99 g, 59.680 mmol, 10.00 equiv) in EtOH (30 mL) was stirred overnight at 80° C. The reaction was quenched with NH₄Cl (aq.) (30 mL) at room temperature. The precipitated solids were collected by filtration and washed with water (3×10 mL). This resulted in 12-10 (1.6 g, 73.60%) as a white solid.

### Synthesis of 12-11

A solution of 12.10 (1.6 g, 4.774 mmol, 1 equiv) and methyl isothiocyanate (701.54 ng, 9.596 mmol, 2.01 equiv) in THF (30 mL) was stirred overnight at room temperature. The resulting mixture was diluted with water (30 mL). The resulting mixture was concentrated under vacuum. The precipitated solids were collected by filtration and washed with water (2×10 mL). This resulted in 12-11 (1.5 g, 71.57%) as a yellow solid.

### Synthesis of 12-12

A solution of 12-11 (1.5 g, 3.674 mmol, 1 equiv) and NaOH (1.47 g, 36.740 mmol, 10.00 equiv) in H₂O (20 mL) was stirred overnight at room temperature. The mixture was acidified to pH 5 with HCl(aq.). The precipitated solids were collected by filtration and washed with water (2×10 mL). This resulted in 12-12 (1.2 g, 75.32%) as a yellow solid.

### Synthesis of 12-13

To a stirred solution of 12-12 (1.2 g, 3.075 mmol, 1 equiv) and NaNO₂ (2.12 g, 30.750 mmol, 10 equiv) in H₂O (20 mL) was added HNO₃ (5 mL, 1 M) dropwise at room temperature. The resulting mixture was stirred for 3 h at room temperature. The mixture was neutralized to pH 7 with saturated NaHCO₃ (aq.). The aqueous layer was extracted with EtOAc (3×10 mL). The residue was purified by reversed-phase flash chromatography with the following conditions: column, C18 silica gel; mobile phase, MeCN in Water (0.1% FA), 30% to 40% gradient in 10 min; detector, UV 254 nm. This resulted in 12-13 (600 mg, 49.03%) as a brown oil.

### Synthesis of 12-14

To a stirred solution of 12-13 (450 mg, 1.256 mmol, 1 equiv) in DCM (15 mL) was added DAST (810.03 mg, 5.024 mmol, 4 equiv) dropwise at 0° C. The resulting mixture was stirred overnight at room temperature. The reaction was quenched with NaHCO₃ (aq.) (100 mL) at room temperature. The aqueous layer was extracted with DCM (3×100 mL). The resulting mixture was concentrated under vacuum. The residue was purified by reversed-phase flash chromatography with the following conditions: column, C18 silica gel; mobile phase, MeCN in Water (0.1% NH₃·H₂O), 5% to 70% gradient in 35 min; detector, UV 254 nm. This resulted in 12-14 (400 mg, 83.98%) as a white solid.

### Synthesis of 12-15

To a stirred solution of 12-14 (500 mg, 1.388 mmol, 1 equiv), Cu₂O (39.73 mg, 0.278 mmol, 0.2 equiv) and MeCN (5 mL) in NH₄₀H (5 mL) was added L-Proline (15.98 mg, 0.139 mmol, 0.10 equiv) at room temperature. The resulting mixture was stirred overnight at 100° C. The resulting mixture was filtered; the filter cake was washed with DCM (3×10 mL). The filtrate was concentrated under reduced pressure. The resulting mixture was concentrated under vacuum. The residue was purified by reversed-phase flash chromatography with the following conditions: column, C18 silica gel; mobile phase, MeCN in Water (0.1% NH₃·H₂O), 5% to 35% gradient in 30 min; detector, UV 254 nm. This resulted in 12-15 (370 mg, 88.16%) as a green solid.

### Synthesis of 12-16

To a stirred solution of 12-15 (360 mg, 1.215 mmol, 1 equiv) and 5-bromo-3-(trifluoromethyl)pyridine-2-carbaldehyde (462.92 mg, 1.823 mmol, 1.5 equiv) in DCE (5 mL) was added STAB (515.01 mg, 2.430 mmol, 2 equiv) at room temperature. The resulting mixture was stirred for overnight at room temperature. The reaction was quenched with sat. NaHCO₃ (aq.) at room temperature. The aqueous layer was extracted with DCM (3×10 mL). The resulting mixture was concentrated under vacuum. The residue was purified by Prep-TLC (DCM/MeOH 15:1) to afford 12-16 (500 mg, 73.17%) as a yellow solid.

### Synthesis of 12-17

To a stirred solution of 12-16 (480 mg, 0.898 mmol, 1 equiv) and pyridine (426.36 mg, 5.388 mmol, 6 equiv) in DCM (5 mL) was added BTC (101.30 mg, 0.341 mmol, 0.38 equiv) at room temperature. The resulting mixture was stirred for 30 min at room temperature.

The reaction was quenched with NaHCO₃ (aq.) (50 mL) at room temperature. The aqueous layer was extracted with DCM (3×50 mL). The resulting mixture was concentrated under vacuum. The residue was purified by Prep-TLC (DCM/MeOH 35:1) to afford 12-17 (420 mg, 79.27%) as a yellow solid.

### Synthesis of 12-18

To a solution of 12-17 (400 mg, 0.714 mmol, 1 equiv) in dioxane (20 mL) was added bis(adamantan-1-yl) (butyl)phosphane (51.19 mg, 0.143 mmol, 0.2 equiv) and TMEDA (165.92 mg, 1.428 mmol, 2 equiv) and Pd(OAc)h (23.6 mg, 0.222 mmol, 0.1 equiv) in a pressure tank. The mixture was purged with nitrogen and then was pressurized to 10 atm with CO/12=1:1 at 80° C. for overnight. The reaction mixture was cooled to room temperature and filtered to remove insoluble solids. The resulting mixture was concentrated under vacuum. The residue was purified by Prep-TLC (DCM/MeOH 10:1) to afford 12-18 (180 mg, 46.03%) as a yellow solid.

### Synthesis of 12-19

To a stirred solution of 12-18 (140 mg, 0.275 mmol, 1 equiv) and (3S)-3-isopropyl-1-methylpiperazine (58.64 mg, 0.413 mmol, 1.5 equiv) in DCE (5 mL) was added tetrakis(propan-2-yloxy)titanium (156.22 mg, 0.550 mmol, 2 equiv) at room temperature. The resulting mixture was stirred overnight at mom temperature. To the above mixture was added STAB (16.49 mg, 0.550 mmol, 2 equiv) at room temperature. The resulting mixture was stirred for additional 3h at room temperature. The reaction was quenched with NaHCO₃ (aq.) at room temperature. The aqueous layer was extracted with DCM (3×20 mL). The resulting mixture was concentrated under vacuum. The residue was purified by reversed-phase flash chromatography with the following conditions: column, C18 silica gel; mobile phase, MeCN in Water (10 mmol/L NH₄HCO₃), 23% to 30% gradient in 10 min; detector. UV 254 nm. This resulted in 12-19 (50 mg, 27.19%) as a yellow solid.

### Synthesis of Compound 12

The 12-19 (50 mg) was purified by Prep-HPLC with the following conditions (Column: Lux 5 um Cellulose-4, 2.12*25 cm, 5 μm; Mobile Phase A: Hex(0.5% 2M NH₃-MeOH), Mobile Phase B: ETOH--HPLC; Flow rate: 20 mL/min; Gradient: 25% B to 25% B in 45 min; Wave Length: 220/254 nm: RT1 (min): 21.66: RT2 (min): 40.81, the first peak is product) to afford Compound 12 (9.9 mg, 20.02%) as a yellow solid.

LC-MS-Compound 12: (ES, m/z): [M+H]⁺ 636

H-NMR-Compound 12: (400 MHz, DMSO, δ ppm): 0.85-0.91 (m, 6H), 1.90-1.98 (m, 2H), 2.13-2.19 (m, 4H), 2.21-2.24 (m, 2H), 2.46-2.50 (m, 1H), 2.76-2.79 (m, 1H), 3.01-3.05 (m, 4H), 3.15-3.17 (m, 2H), 3.33-3.48 (m, 2H), 3.78-3.82 (m, 2H), 6.09-6.21 (d, 1H), 6.96 (s, 1H), 7.09-7.10 (m, 1H), 7.37-7.39 (s, 1H), 7.56 (s, 1H), 7.56 (s, 1H), 7.68 (s, 1H), 7.78-7.80 (m, 1H), 5.34 (s, 1H).

### Example 13. Synthesis of Compound 13

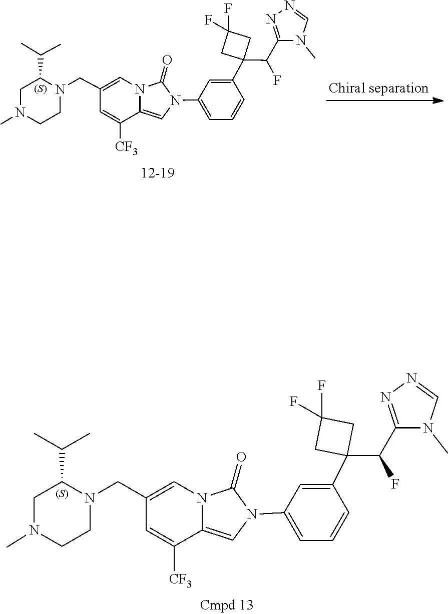

The 12-19 (50 mg) was purified by Prep-HPLC with the following conditions (Column: Lux Sum Cellulose-4, 2.12*25 cm, 5 μm; Mobile Phase A: Hex(0.5% 2M NH-MeOH), Mobile Phase B: ETOH--HPLC; Flow rate: 20 mL/min; Gradient: 25% B to 25% B in 45 min; Wave Length: 220/254 nm; RT1 (min): 21.66; RT2 (min): 40.81, the second peak is product) to afford Compound 13 (11.1 mg, 22.52%) as a yellow solid.

LC-MS-Compound 13: (ES, m/z): [M+H]⁺ 636

H-NMR-Compound 13: (400 MHz, DMSO, δ ppm): 0.85-0.9I((m, 6H), 1.90-1.98 (m, 21), 2.13-2.19 (m, 4H), 2.21-2.24 (m, 2H), 2.30 (m, 1H), 2.54 (m, 1H), 2.76-2.79 (m, 1H), 3.01-3.05 (m, 41), 3.15-3.17 (m, 2H), 3.33-3.48 (in, 2H-1), 3.78-3.82 (m, 2H), 6.09-6.20 (d, 1H), 6.96 (s, 1H), 7.08-7.10 (m, 1H), 7.37-7.39 (s, 1H), 7.41-7.43 (m, 1H), 7.56 (s, 1 H), 7.56 (s, 1H), 7.68 (s, 1H), 7.78-7.80 (m, 1H), 5.34 (m, 1H).

### Example 14. Synthesis of Compound 14

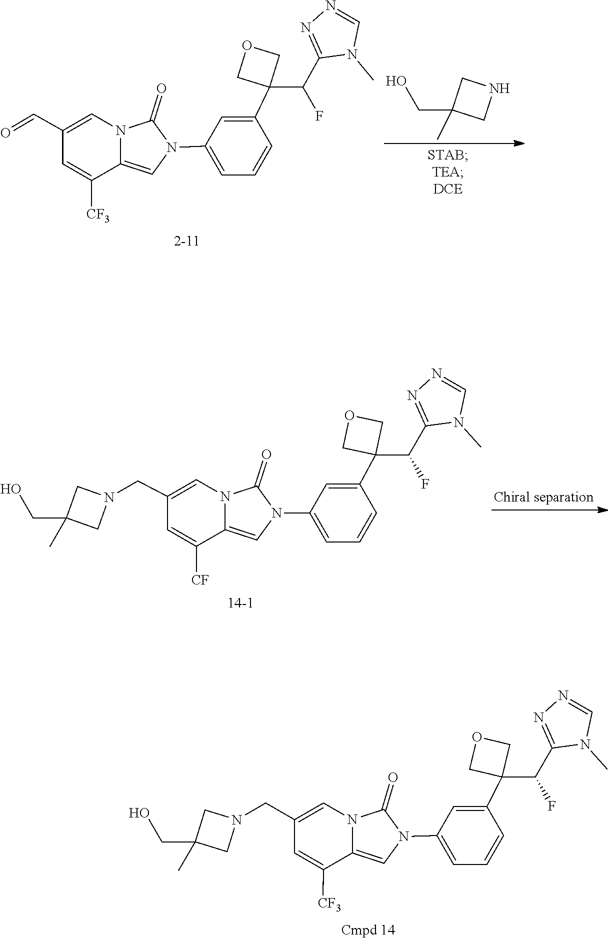

### Synthesis of 14-1

To a stirred solution of 2-11 (300 mg, 0.631 mmol, 1 equiv) and (3-methylazetidin-3-yl)methanol (127.66 mg, 1.262 mmol, 2 equiv) in DCE (10 mL) was added TEA (127.71 mg, 1.262 mmol, 2 equiv) at room temperature. The resulting mixture was stirred for 1h at room temperature. To the above mixture was added STAB (267.49 mg, 1.262 mmol, 2 equiv) at room temperature. The resulting mixture was stirred for additional 3h at room temperature. The reaction was quenched with Na-1CO. (aq.) (20 mL) at room temperature.

The aqueous layer was extracted with DCM (3×20 mL). The resulting mixture was concentrated under vacuum. The crude product was purified by Prep-HPLC with the following conditions (Column: Select CSH C18 OBD Column 30*150 mm 5 μm; Mobile Phase A: Water(0.1% FA), Mobile Phase B: ACN: Flow rate: 60 mL/min mL/min; Gradient: 5% B to 25% B in 7 min: Wave Length: 254 nm/220 nm nm; RT1 (min): 6.65) to afford 14-1 (110 mg, 30.16%) as a yellow solid.

### Synthesis of Compound 14

The 14-1 (110 mg) was purified by Prep-HPLC with the following conditions (Column: CHIRALPAK IG, 2*25 cm, 5 μm; Mobile Phase A: Hex (0.5% 2M NH₃-MeOH). Mobile Phase B: EtOH:DCM=1:1--HPLC; Flow rate: 20 mL/min: Gradient: 40% B to 40% B in 27 min: Wave Length: 220/254 nm; RT1 (min): 15.22; RT2 (min): 21.64, the first peak is product) to afford Compound 14 (36.6 mg, 34.43%) as a yellow solid.

LC-MS-Compound 14: (ES, m/z): [M+H]⁺ 561

H-NMR-Compound 14: (400 MHz, DMSO, δ ppm): 1.15 (s, 3H), 2.66-2.67 (m, 2H), 3.09-3.13 (m, 2H), 3.15-3.19 (m, 3H), 3.20-3.27 (m, 2H), 3.42 (s, 1H), 4.73-4.75 (m, 1H), 4.84-4.86 (s, 1H), 5.17-5.19 (m, 2H), 5.33-5.35 (m, 1H), 6.22-6.33 (d, 1H), 6.99 (s, 1H), 7.12-7.14 (m, 1H), 7.33 (s, 1H), 7.41-7.45 (m, 1H), 7.56 (s, 1H), 7.66 (s, 1H), 7.77-7.78 (m, 1H), 7.79-7.80 (m, 1H), 5.14 (s, 1H).

### Example 15. Synthesis of Compound 15

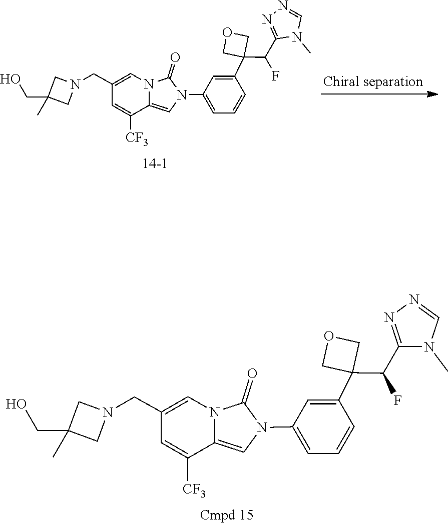 [SMILES: Cn1cnnc1C(F)C1(c2cccc(-n3cc4c(C(F)(F)F)cc(CN5CC(C)(CO)C5)cn4c3=O)c2)COC1 ; Cn1cnnc1[C@@H](F)C1(c2cccc(-n3cc4c(C(F)(F)F)cc(CN5CC(C)(CO)C5)cn4c3=O)c2)COC1]

The 14-1 (110 mg) was purified by Prep-HPLC with the following conditions (Column: CHIRALPAK IG, 2*25 cm, 5 μm; Mobile Phase A: Hex (0.5% 2M NH₃-MeOH), Mobile Phase B: EtOH:DCM=1:1--HPLC; Flow rate: 20 mL/min: Gradient: 40% B to 40% B in 27 min; Wave Length: 220/254 nm; RT1 (min): 15.22; RT2 (min): 21.64, the second peak is product) to afford Compound 15 (40.4 mg, 37.58%) as a yellow solid.

LC-MS-Compound 15: (ES, m/z): [M+H]⁺ 561

H-NMR-Compound 15: (400 MHz, DMSO-d6, 6 ppm): 1.23 (s, 3H), 2.67-2.87 (m, 2H), 3.12-3.14 (m, 2H), 3.27-3.31 (m, 3H), 3.37 (s, 1H), 3.75-4.84 (m, 1H), 4.85-4.86 (m, 1H), 5.17-5.21 (m, 2H), 5.33-5.35 (in, 11H), 6.21-6.33 (d, 1H), 8.99 (s, 11-H), 7.12-7.14 (m, 11H), 7.33 (s, 1H), 7.41-7.45 (m, 1H), 7.56 (s, 1H), 7.66 (s, 1H), 7.77-7.78 (in, 1H), 7.79-7.80 (m, 1H), 8.37 (s, 1H).

### Example 16. Synthesis of Compound 16

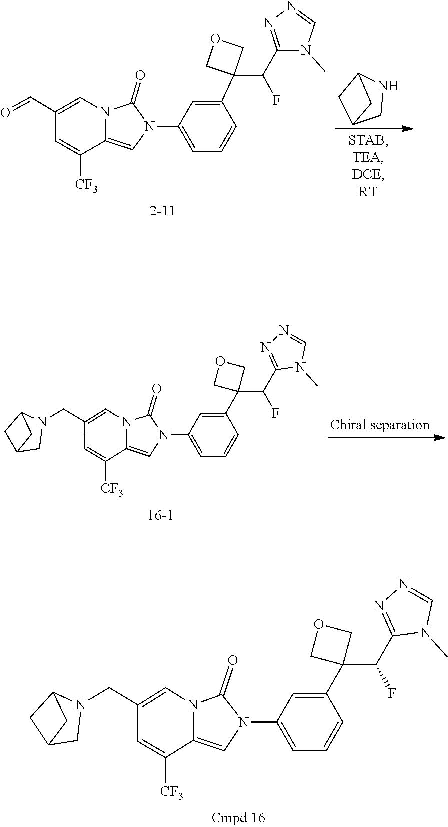

### Synthesis of 16-1

To a stirred solution of 2-11 (300 mg, 0.631 mmol, 1 equiv) and 2-azabicyclo 12.1.11 hexane (78.69 mg, 0.947 mmol, 1.5 equiv) in DCE (3 mL) was added TEA (191.57 mg, 1.893 mmol, 3 equiv) at room temperature. The resulting mixture was stirred for 1h at room temperature. To the above mixture was added STAB (401.23 mg, 1.893 mmol, 3 equiv) at room temperature. The resulting mixture was stirred for additional 3h at room temperature. The reaction was quenched with NaHCO₃ (aq.) (10 mL) at room temperature.

The aqueous layer was extracted with DCM (3×10 mL). The resulting mixture was concentrated under vacuum. The residue was purified by reversed-phase flash chromatography with the following conditions: column, C18 silica gel; mobile phase, MeCN in Water (10 mmol/L NH₄HCO₃), 20% to 30% gradient in 10 min; detector, UV 254 nm. This resulted in 16-1 (150 mg, 42.50%) as a yellow solid.

### Synthesis of Compound 16

The 16-1 (150 mg) was purified by Prep-HPLC with the following conditions (Column: CHIRALPAK IC, 2*25 cm, 5 μm: Mobile Phase A: Hex (0.5% 2M NH₃-MeOH), Mobile Phase B: MEOH:DCM=1:1--HPLC; Flow rate: 20 mL/min; Gradient: 60% B to 60% B in 15 min: Wave Length: 220/254 nm; RT1 (min): 9.99; RT2 (min): 11.75, the first peak is product) to afford Compound 16 (54.7 mg, 38.16%) as a yellow solid.

LC-MS-Compound 16: (ES, m/z): [M+H]⁺ 543

H-NMR-Compound 16: (400 MHz, DMSO, δ ppm): 1.35-1.43 (m, 2H), 1.62-1.76 (m, 2H), 2.61-2.64 (m, 2H), 2.66-2.72 (m, 1H), 3.20-3.27 (m, 3H), 3.42-3.52 (m, 3H), 4.84-4.87 (m, 1H), 5.17-5.21 (m, 2H), 5.33-5.35 (m, 1H), 6.22-6.33 (d, 2H), 7.12-7.14 (m, 2H), 7.31 (s, 1H), 7.41-7.45 (m, 1H), 7.56 (s, 1H), 7.68 (s, 1H), 7.74-7.80 (m, 1H), 5.36 (s, 1H).

### Example 17. Synthesis of Compound 17

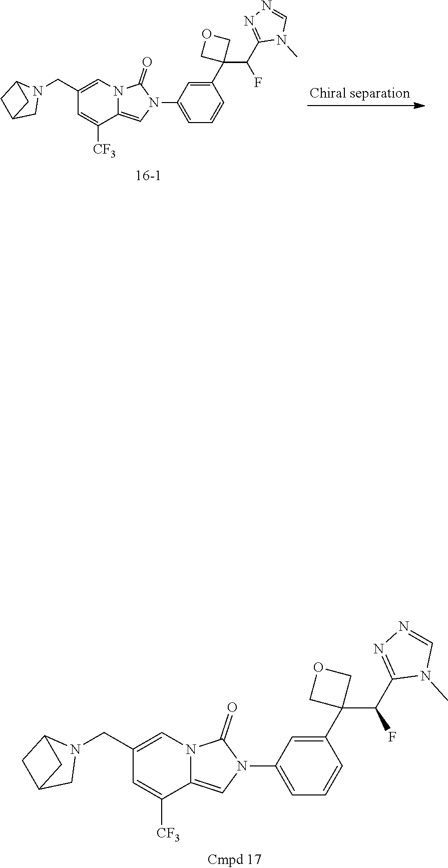 [SMILES: Cn1cnnc1C(F)C1(c2cccc(-n3cc4c(C(F)(F)F)cc(CN5CC6CC5C6)cn4c3=O)c2)COC1 ; Cn1cnnc1[C@@H](F)C1(c2cccc(-n3cc4c(C(F)(F)F)cc(CN5CC6CC5C6)cn4c3=O)c2)COC1]

The 16-1 (150 mg) was purified by Prep-HPLC with the following conditions (Column: CHIRALPAK IC, 2*25 cm, 5 μmm: Mobile Phase A: Hex (0.5% 2M NI-h-MeOH), Mobile Phase B: MEOH:DCM=1:1--HPLC; Flow rate: 20 mL/min: Gradient: 60% B to 60% B in 15 min; Wave Length: 220/254 nm; RT1 (min): 9.99; RT2 (min): 11.75, the second peak is product) to afford Compound 17 (64.5 mg, 44.72%) as a yellow solid.

LC-MS-Compound 17 (ES, m/z): [M+H]⁺ 543

H-NMR-Compound 17 (400 MHz, DMSO, δ ppm): 1.51 (s, 2H), 1.68-1.75 (m, 2H), 2.63-2.66 (m, 2H), 2.71-2.73 (m, 1H), 3.17-3.29 (m, 3H), 3.45-3.54 (m, 3H), 4.84-4.87 (m, 1H), 5.17-5.22 (m, 2-11, 5.33-5.35 (m, 1H), 6.22-6.33 (d, 2H), 7.12-7.14 (m, 2H), 7.31 (s, 1H), 7.41-7.45 (m, 1H), 7.56 (s, 11H), 7.68 (s, 1H), 7.74-7.80 (m, 11H), 5.36 (s, 1H).

### Example 18. Synthesis of Compound 18

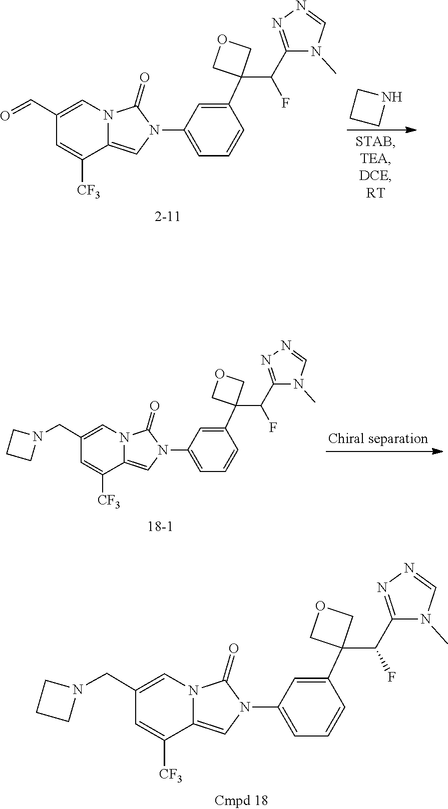

### Synthesis of 18-1

To a stirred solution of 2-11 (300 mg, 0.631 mmol, 1 equiv) and azetidine (54.04 mg, 0.947 mmol, 1.5 equiv) in DCE (3 mL) was added TEA (95.79 mg, 0.947 mmol, 1.5 equiv) at room temperature. To the above mixture was added STAB (401.23 mg, 1.893 mmol, 3 equiv) at room temperature. The resulting mixture was stirred for additional 3h at room temperature. The reaction was quenched with NaHCO₃ (aq.) at room temperature. The aqueous layer was extracted with DCM (3×10 mL). The resulting mixture was concentrated under vacuum. The residue was purified by reversed-phase flash chromatography with the following conditions: column. CIS silica gel; mobile phase, MeCN in Water (00 mmol/L NH₄HCO₃), 20% to 30% gradient in 10 min; detector, UV 254 nm. This resulted in 18-1 (130 mg, 38.29%) as a yellow solid.

### Synthesis of Compound 18

The 18-1 (130 mg) was purified by Prep-HPLC with the following conditions (Column: CHIRALPAK IC, 2*25 cm, 5 μm; Mobile Phase A: Hex(0.5% 2M NH₃-MeOH), Mobile Phase B: EtOH:DCM=1:1--HPLC; Flow rate: 20 mL/min; Gradient: 60% B to 60% B in 17 min; Wave Length: 220/254 nm; RT1 (min): 11.99; RT2 (min): 14.58, the first peak is product) to afford Compound 18 (32.6 mg, 26.37%) as a yellow solid.

LC-MS-Compound 18: (ES, m/z): [M+H]⁺ 517

H-NMR-Compound 18: (400 MHz, CD-OD, δ ppm): 2.10-2.18 (m, 2H), 3.12-3.19 (m, 4H), 3.29-3.37 (m, 3-3.43-3.48 (m, 2H), 5.00-5.02 (in, 1H) 5.22-5.24 (m, 11K), 5.29-5.31 (m, 1H), 5.45-5.47 (in, 1H), 5.24-6.35 (d, 1H), 7.01 (s, 1H), 7.08-7.11 (m, 2H), 7.44-7.45 (m, 1H), 7.47-7.49 (m, 11H), 7.63-7.65 (m, 2H), 8.32 (s, 1H).

### Example 19. Synthesis of Compound 19

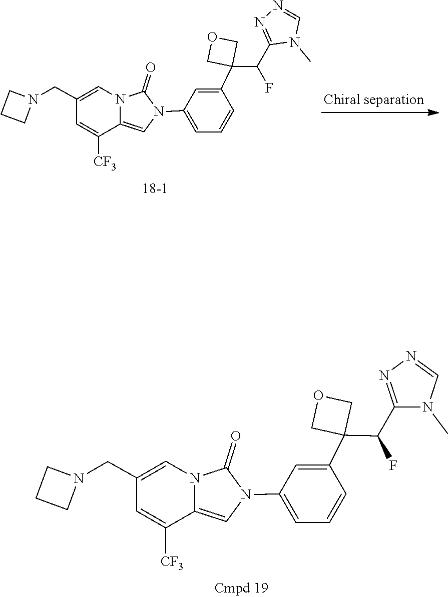 [SMILES: Cn1cnnc1C(F)C1(c2cccc(-n3cc4c(C(F)(F)F)cc(CN5CCC5)cn4c3=O)c2)COC1 ; Cn1cnnc1[C@@H](F)C1(c2cccc(-n3cc4c(C(F)(F)F)cc(CN5CCC5)cn4c3=O)c2)COC1]

The 18-1 (130 mg) was purified by Prep-HPLC with the following conditions (Column: CHIRALPAK IC, 2*25 cm, 5 μm; Mobile Phase A: Hex (0.5% 2M NH₃-MeOH), Mobile Phase B: EtOH:DCM=1:1--HPLC; Flow rate: 20 mL/min; Gradient: 60% B to 60% B in 17 min; Wave Length: 220/254 nm; RT1 (min): 11.99; RT2 (min): 14.58, the second peak is product) to afford Compound 19 (39 mg, 31.55%) as a yellow solid.

LC-MS-Compound 19: (ES, m/z): [M+H]⁺ 517

H-NMR-Compound 19: (400 MHz, CD3OD, 6 ppm): 2.11-2.18 (m, 2H), 3.12-3.19 (m, 4H), 3.29-3.36 (m, 3H) 3.43-3.48 (m, 2H), 5.00-5.02 (m, 1H), 5.22-5.24 (m, 1H), 5.27-5.31 (m, 1H), 5.45-5.47 (m, 1H), 6.24-6.35 (d, 1H), 7.01 (s, 1H), 7.08-7.11 (m, 2H), 7.44-7.45 (m, 1H), 7.47-7.49 (m, 1H), 7.63-7.65 (m, 2H), 8.32 (s, 1H).

### Example 20. Synthesis of Compound 20

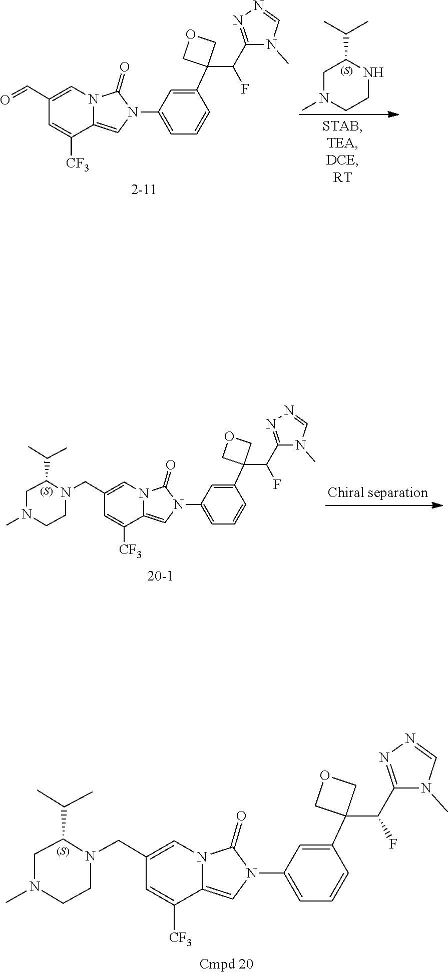

### Synthesis of 20-1

To a stirred solution of 2-11 (300 mg, 0.631 mmol, 1 equiv) and (3S)-3-isopropyl-1-methylpiperazine (134.64 mg, 0.947 mmol, 1.5 equiv) in DCE (10 mL) was added TEA (127.71 mg, 1.262 mmol, 2 equiv) at mom temperature. To the above mixture was added STAB (267.49 mg, 1.262 mmol, 2 equiv) at room temperature. The resulting mixture was stirred for additional overnight at room temperature. The reaction was quenched with NaHCO₃ (aq.) (30 mL) at room temperature. The aqueous layer was extracted with DCM (3×10 mL). The resulting mixture was concentrated under vacuum. The residue was purified by reversed-phase flash chromatography with the following conditions: column, C18 silica gel; mobile phase, MeCN in Water (10 mmol/L NH₄HCO₃), 20% to 30% gradient in 10 min; detector. UV 254 nm. This resulted in 20-1 (140 mg, 35.40%) as a yellow solid.

### Synthesis of Compound 20

The 20-1 (140 mg) was purified by Prep-HPLC with the following conditions (Column: CHIRALPAK IC, 2*25 cm, 5 μm: Mobile Phase A: Hex (0.5% 2M NH₃-MeOH), Mobile Phase B: ETOH:DCM=1:1--HPLC; Flow rate: 20 mL/min: Gradient: 60% B to 60% B in 17 min: Wave Length: 220/254 nm; RT1 (min): 10.12; RT2 (min): 12.58; the first peak is product) to afford Compound 20 (43.5 mg, 31.20%) as a yellow solid.

LC-MS-Compound 20: (ES, m/z): [M+H]⁺ 602

H-NMR-Compound 20: (400 MHz, DMSO, 5 ppm): 0.86-0.88 (m, 3H), 0.89-0.91 (m, 3H), 1.99-2.00 (m, 2H), 2.16-2.20 (m, 3H), 2.21-2.25 (m, 2H), 2.49-2.50 (m, 2H), 2.80-2.82 (m, 1H), 3.02-3.04 (m, 1H), 3.27-3.34 (m, 3H), 3.78-3.82 (m, 1H), 4.84-4.86 (m, 1H), 5.17-5.21 (m, 2H), 5.33-5.35 (m, 1H), 6.22-6.33 (d, 1H), 6.95 (s, 1-1), 7.12-7.14 (m, 1H), 7.34 (s, 1H), 7.41-7.45 (m, 1H), 7.56 (s, 1H), 7.68 (s, 1H), 7.77-7.79 (m, 111), 8.37 (s, 1H).

### Example 21. Synthesis of Compound 21

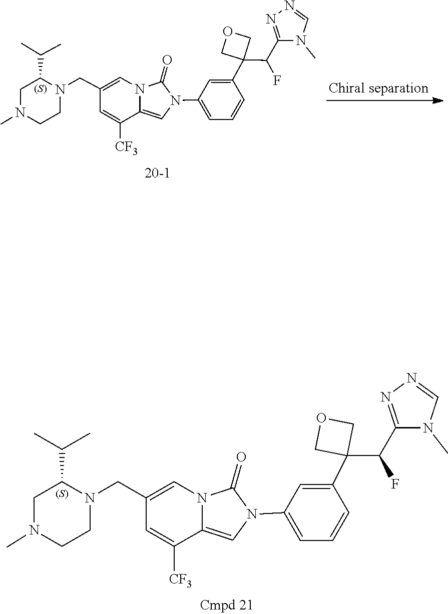

The 20-1 (140 mg) was purified by Prep-HPLC with the following conditions (Column: CHIRALPAK IC, 2*25 cm, 5 p-m: Mobile Phase A: Hex (0.5% 2M NH₃-MeOH), Mobile Phase B: ETOH:DCM=1:1--HPLC; Flow rate: 20 mL/min; Gradient: 60% B to 60% B in 17 min: Wave Length: 220/254 nm: RT(min): 10.12; RT2 (min): 12.58, the second peak is product) to afford Compound 21 (30.8 mg, 23.07%) as a yellow solid.

LC-MS-Compound 21: (ES, m/z): [M+H]⁺ 602

H-NMR-Compound 21: (400 MHz, DMSO, 5 ppm): 0.86-4).91 (m, 61), 1.99-2.00 (m, 2H), 2.17-2.32 (m, 61H), 2.49-2.50 (m, 2H), 2.78-2.81 (m, 1H), 3.02-3.05 (m, 1H), 3.27-3.33 (n, 3H), 3.78-3.82 (m, 1H), 4.84-4.86 (m, 1H), 5.17-5.21 (m, 2H), 5.33-5.35 (m, 1H), 5.22-6.33 (d, 11H), 6.95 (s, 11H), 7.11-7.13 (m, 1H), 7.35 (s, 1H), 7.41-7.45 (m, 1H), 7.56 (s, 1H), 7.68 (s, 11H), 7.77-7.80 (m, 11H), 8.37 (s, 11H).

### Example 22. IC50 Screening

### Experiment Procedure

a) Thaw UBE1, UBCH5b, CBL-B, Biotin-Ubiquitin, CBL-B assay buffer, and ATP on ice. Aliquot each protein, CBL-B assay buffer, and ATP into single-use aliquots and stored at −80° C. immediately.
b) Transfer 100 nL test compound (10 mM stock solution, start at 100 uM, 1:3 dilution, 11 points) and then backfill 100 nL DMSO to each well designated for the “Test compound” by Echo.
c) Transfer 200 nL DMSO to each well designated for the “High Control”, “Low Control” by Echo.
d) Carefully calculate the amount of proteins needed. Prepare appropriate amounts of diluted proteins; dilute only the amount required for the assay. Do not store diluted proteins. Keep the diluted reagents on ice until use.
e) Add 2.5 μl diluted CBL-B (40 nM) for the “High Control” and “Test compound”, the final concentration is 10 nM. For the “Low Control”, add 2.5 μL buffer each well. Pre-incubate for 15 min.
f) Centrifuge at 1000 rpm for 1 minute and shake plate at a speed of 600 rpm for 5 minutes.
g) Prepare the Master Mixture (E1E2) using diluted reagents: N wells×(1 μl Biotin-Ub+0.5 μl diluted UBE1+1 μl diluted UBCH5+5 μl ATP). Pre-incubation for 10 min.
h) Add master mixture to each well. Centrifuge at 1000 rpm for 1 minute and shake plate at a speed of 600 rpm for 5 minutes.
i) Incubate the reaction at 30° C. for 20 minutes. Cover the plate with a plate sealer.
j) Dilute Mab Anti GST-Tb cryptate donor (1:100) and Streptavidin-d2 acceptor (1:100) using PPI-Terbium detection buffer. Prepare only the amount required for the assay Add 5 μL diluted donor and 5 μL acceptor mixture into each well. Centrifuge at 1000 rpm for 1 minute and shake plate at a speed of 600 rpm for 5 minutes. Incubate at room temperature for one hour.
k) Read the fluorescent intensity with EnVison2104 (PerkinElmer Life Sciences). Blank value is subtracted from all other values. Tb-donor emission should be measured at 615 nM followed by dye-acceptor emission at 665 nM.

### Plate Map for IC50 Screen (Dose Titration of 11 Doses for 2 Reps)

a) Dilute cpds in 384 well dilution plate according to the dilution plate map
b) Cpds starting conc: 10 mM
c) Final cpd conc in assay: 100000, 33333.3, 11111.1, 3703.7, 1234.57, 411.52, 137.17, 45.72, 15.24, 5.08, 1.69 and 0 nM.
d) High control wells (HC): DMSO vehicle, with Cbl-b
e) Low control wells (LC): DMSO vehicle, No Cbl-b

### Data Analysis

### IC50 Screening Data Analysis

a) Calculate average of HC and LC samples
b) Calculate plate Z′ Factor:

$${Z}^{′} = 1 - 3 \times \frac{{STD}_{HC} + {STD}_{LC}}{{AVG}_{HC} - {AVG}_{LC}}$$

The Z′ should be >0.5. Otherwise, consider the plate failed QC and repeat the experiment.
c) Calculate percent vehicle of each test well:

$$\% Vehicel = 100 \times \frac{Test cpd - {Ave}_{LC}}{{Ave}_{HC} - {Ave}_{LC}}$$

d) Fit the cpd IC50 from non-linear regression equation (Equation:201, XLfit5.3.1.3):

$$Y = Bottom + ( Top - Bottom ) / ( 1 + 10 \hat{} ( ( Log IC 50 - X ) * HillSlope ) )$$

$$X : Log of cpd concentration \\ Y : Percent inhibition ( \% inh ) \\ Top and Bottom : Plateaus in same units as Y \\ log IC 50 : same log units as X$$

$$HillSlope : Slope Factor or Hill slope$$

### Example 23. Cbl-b and C-cbl LCK Ub TR-FRET Assay

Compounds are 3-fold serially diluted in DMSO in a 384-well polypropylene plate (e.g., #P-05525-BC; Labcyte) to generate a source plate with 10 concentrations of each compound, top concentration=2 mM. 80 nL of DMSO or compounds are transferred to each well of a black 384-well ProxiPlate (e.g., #6008260; PerkinElmer) using a Labcyte Echo, 1×assay buffer (50 mM HEPES pH7.0, 100 mM NaCl, 0.01% BSA, 0.01% Triton-X100, 1 mM DTT), 2×enzyme solution (16 nM Biotin-Cbl-b or 12 nM Biotin-c-Cbl in 1×assay buffer), 2×kinase mixture (120 nM His-LCK, 1 mM ATP, 10 mM MgCl2 in assay buffer) and 2.33×detection mixture (4.66×solution 1:163 nM Anti-HA-D2 antibody (e.g., #610HADAB:PerkinElmer), 27.96 nM Streptavidin-EU (e.g., #AD0062; PerkinElmer), 1.398 mM EDTA in 1×assay buffer+4.66×solution 2:2.796 pM UBE2D2/Methylated-HA-Ubiquitin thioester adduct (e.g., BostonBiochem) in 1×assay buffer) are prepared. 4 μL of 2×enzyme solution are added to each well containing compound, briefly centrifuged to mix, and incubated for 60 min at room temperature. 4 μL of 2×kinase mixture is added, briefly centrifuged to mix, and incubated for 90 min. at room temperature. 6 μL of detection mixture is added to all wells and briefly centrifuged before incubating for 20 min at room temperature. Plates am read for TR-FRET using an Envision at excitation 340 nm, emission at 615 and 665 nm, 4 flashes per well. IC50 is generated using no LCK as the low control and DMSO as the high control.

Compound IC_{50S} are classified into grades A through C, as follows: A indicates 0-100 nM. B indicates 101 nM-500 nM, and C indicates 501′-10,000 nM.

TABLE 2

| | | CBL-B_TR- |
|---|---|---|
| Cmpd | | FRET_AVG_IC50 |
| No. | Structure | (nM) |
| 1 | 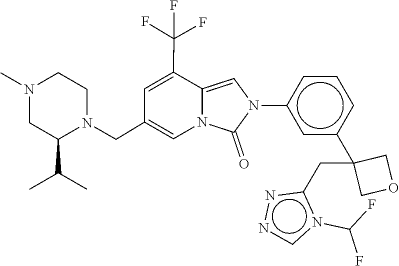 | A |
| 2 | 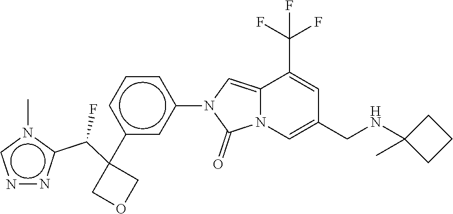 | C |
| 3 | 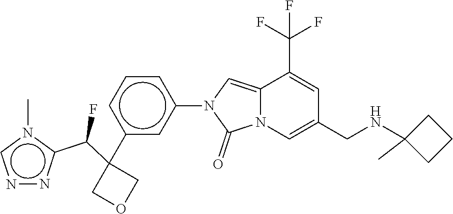 | C |
| 4 | 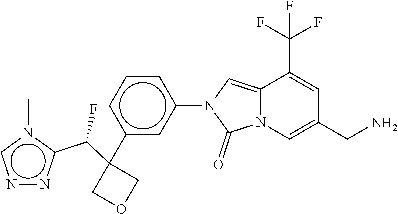 | B |
| 5 | 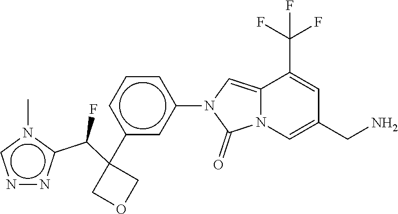 | B |
| 6 | 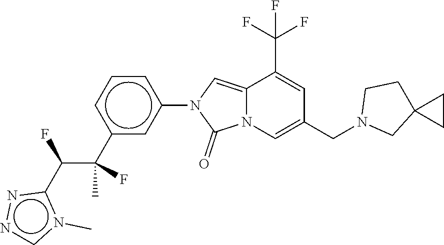 | A |
| 7 | 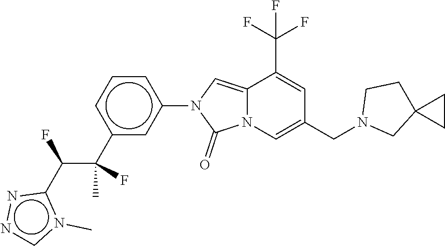 | B |
| 8 | 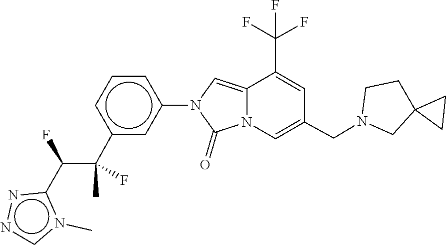 | B |
| 9 | 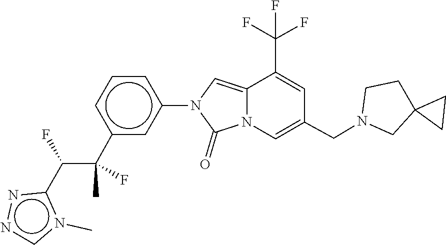 | C |
| 10 | 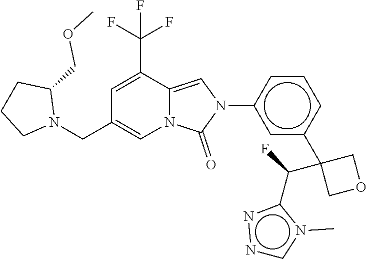 | C |
| 11 | 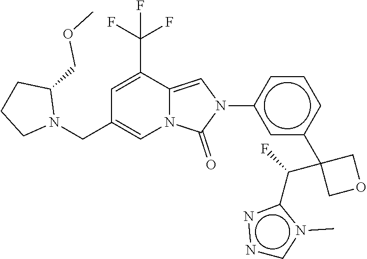 | B |
| 12 | 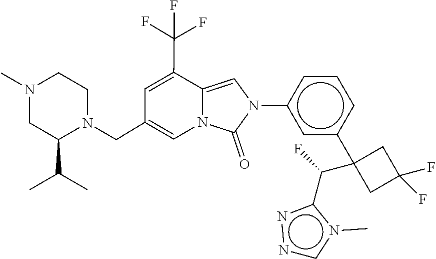 | A |
| 13 | 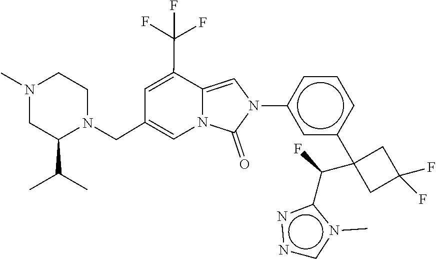 | A |
| 14 | 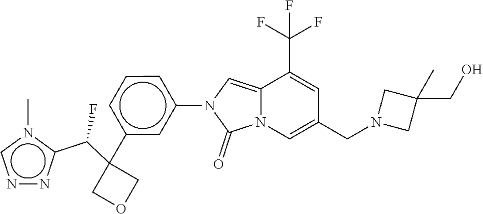 | B |
| 15 | 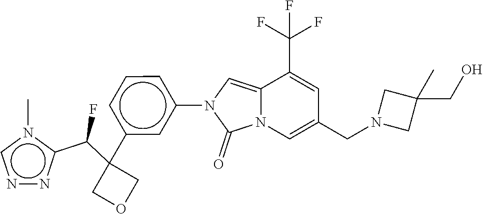 | A |
| 16 | 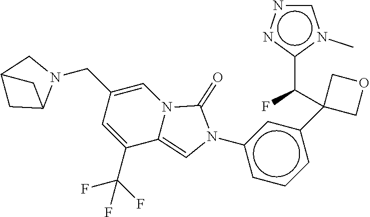 | A |
| 17 | 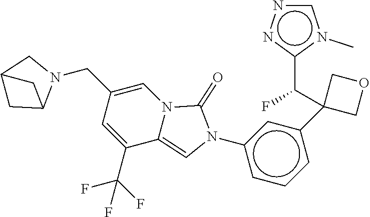 | B |
| 18 | 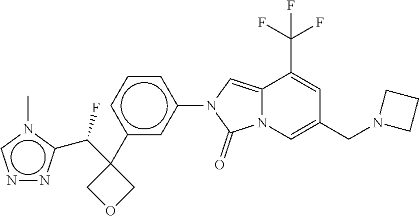 | A |
| 19 | 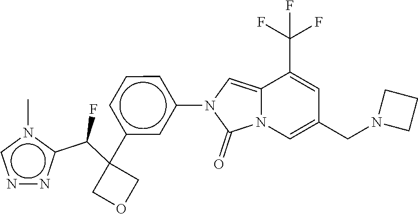 | B |
| 20 | 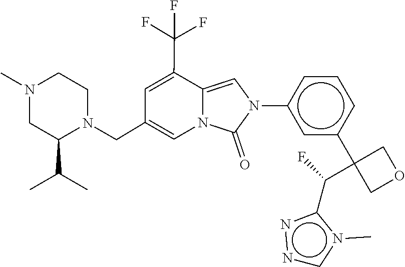 | A |
| 21 | 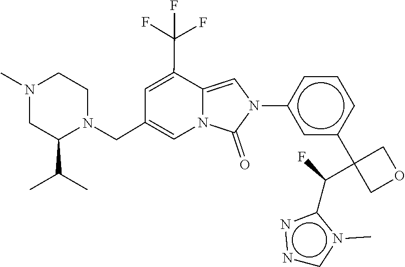 | A |

### Example 24. PBMC IL-2 Assay

Immune response to compounds described herein can be assessed via a PBMC IL-2 assay, conducted according to the following protocol. PBMCs (#A19K379053, A19K261022; TPCS) are thawed into complete medium: 1640 medium (#2085568; Gibco), 10% FBS (#SH30084.03; HyClone), and 1×pen/strep. Compounds are 3-fold serially diluted in DMSO in a 384-well polypropylene plate (#P-05525-BC: Labcyte) using the Tecan EVO to generate a source plate with 10 concentrations of each compound, top concentration=4 mM. Compounds are dispensed into a 96-well plate (#6005680; PerkinElmer) using a Labcyte Echo; final dispensed volume of each control and compound is 1000 nL (final DMSO=0.5%). After recovery overnight, cells are seeded at 2×10⁵ cells/well into 96-well plates containing compounds and incubated at 37′C, 5% CO₂ for 30 min. Cells are stimulated by adding 20 μL/well 1/10 TransAct (#130-111-160; Miltenyi) diluted in complete medium, placed on a shaker for 2 min at 600 rpm, and incubated for 24 h at 37° C., 5% CO₂. Plates are centrifuged at 1200 rpm for 5 min and 120 μL cell supernatant collected. Supernatants are diluted 10-fold and IL-2 concentrations of each sample are determined using the IL-2 MSD kit (#K151 AHB-4: MSD) per the manufacturer's instructions.

### Example 25. Liver Microsomes Metabolic Stability Assays

Metabolic stability of test compounds can be evaluated in pooled rat, mouse, dog, and cynomolgus monkey liver microsomes (BD Biosciences, San Jose, CA), according to the following protocol. The incubation conditions are as follows: 1 pM of the tested compound. 1 mM NADPH, 0.5 mg/mL microsomal protein in 0.1 M potassium phosphate buffer (pH 7.4).

Following a 5-minute pre-incubation period, the enzymatic reactions are initiated by the addition of NADPH and test compound to the microsomes diluted in phosphate buffered saline. The mixtures are incubated at 37° C. for 0, 20, 40, and 60 min.

Compound concentrations are assessed by LC-MS/MS. Intrinsic clearance based upon microsomal stability data is determined using a substrate depletion method and scaled to hepatic clearance using the well-stirred model (Obach, R. S.: Baxter, J. G.; Liston, T. E.; Silber, B. M.; Jones, B. C.; MacIntyre, F.; Rance, D. J.; Wastall, P., “The Prediction of Human Pharmacokinetic Parameters from Prelinical and in vitro Metabolism Data”, J. Pharmacol. Exp. Thee. (1997), 283 (1), 46-58).

### Example 26. Hepatocyte Metabolic Stability Assays

Metabolic stability assays of test compounds can be evaluated in cryopreserved pox)led rat, mouse, dog, and cynomolgus monkey hepatocytes (CellzDirect: Durham, NC, USA), according to the following protocol. Membrane integrity of the cells is assessed by trypan blue exclusion. Test compounds (1.0 pM with 0.1% dimethylsulfoxide) are incubated with cells (0.5 million cells/mL) at 37° C. in a 95% air/5% CO₂ atmosphere for 0, 20, 40, or 60 minutes. Concentrations of test compounds in hepatocyte incubations are determined by LC/MS/MS. Intrinsic clearance is determined using a substrate depletion method and scaled to hepatic clearance using the well-stirred model as described above for the liver microsomes metabolic stability assays.

### Example 27. In Vitro Plasma Protein Binding

In vitro plasma protein binding can be determined in pooled mouse, rat, and human plasma (Bioreclamation, Inc., Hicksville, NY) by equilibrium dialysis using a Rapid Equilibrium Dialysis (RED) device (Pierce Biotechnology/Thermo Fisher Scientific; Rockford, IL) with a molecular weight cut-off of 8000 Daltons. Test compounds are added to plasma. Plasma samples are equilibrated with phosphate-buffered saline at 37° C. for 4 hours. Compound concentrations in post-dialysis plasma and buffer samples were measured by LC-MS/MS. The percent unbound fraction in plasma for each compound is calculated by dividing the compound concentration in the post-dialysis buffer by that measured in the post-dialysis plasma and multiplying by 100%.

### Example 28. In Vitro Permeability Assay in gMDCK (Madin-Darby Canine Kidney)

### Cells

The permeability of test compounds can be determined in gMDCK cells (American Type Culture Collection; Manassas, VA). Four days prior to use, MDCK cells are seeded at a density of 2.5*10 cells/mL in 24 well plates. Compounds are dissolved in transport buffer consisting of Hank's Balanced Salt Solution with 10 mM HEPES (Invitrogen Corporation, Grand Island, NY) at a concentration of 10 pM, and permeability is assessed in the apical to basolateral (A-B) and basolateral to apical (B-A) directions following a 3 hour incubation.

Lucifer Yellow (Sigma Aldrich, St. Louis, MO) is used as the cell monolayer integrity marker. Test compound concentrations in the donor and receiving compartments are determined by LC-MS/MS. The apparent permeability (Papp) of test compounds is determined as follows:

$$Papp = ( dQ / dt ) * ( 1 / ACo )$$

Where dQ/dt is the rate of compound appearance in the receiver compartment, Q is the quantity of compound), Co is the concentration in the donor compartment and A is the surface area of the insert. Efflux ratio is calculated as Pₐₚₚ, B-A/Papp, A-B.

### Example 29. Reversible CYP Inhibition

Reversible CYP inhibition by compounds described herein can be measured by protocols described by Halladay, J. S.; Delarosa, E. M.; Tran, D.; Wang, L.; Wong, S.; Khojasteh, S. C., “High-Throughput, 384-Well, LC-MS/MS CYP Inhibition Assay Using Automation. Cassette-Analysis Technique, and Streamlined Data Analysis”, Drug. Metab. Lett. 2011, 5 (3), 220.230, incorporated herein by reference.

### Example 30. CYP3A Time-Dependent Inhibition (TDI)

Time-dependent inhibition by compounds described herein can be measured by various methods. Exemplary such protocols for CYP3A automated AUC shift dilution TDI assay and definitive Ki/Kinact TDI assay are described by Kenny, J. R.; Mukadam, S.; Zhang, C.; Tay, S.; Collins, C.; Galetin, A.; Khojasteh, S. C., “Drug-Drug Interaction Potential of Marketed Oncology Drugs: in vitro Assessment of Time-Dependent Cytochrome P450 Inhibition. Reactive Metabolite Formation and Drug-Drug Interaction Prediction,” Pharm. Res. 2012, 29 (7), 1960-1976.

### Example 31. In Vivo Pharmacokinetics (PK)

The pharmacokinetics of test compounds can be evaluated following a single intravenous bolus (IV) of solution at a dose of 0.2-1 mg/kg and oral administration (PO) of solution/suspension at doses of 1-5 mg/kg in male cynomolgus monkey, beagle dogs, Sprague Dawley rats, or CD-1 mice using a parallel study design. Blood samples for the IV dose group are collected prior to administration (predose) and at 0.033, 0.083, 0.25, 0.5, 1, 3, 6, 9 and 24 hours post dose. Blood samples for PO dose groups are collected prior to administration (predose) and at 0.083, 0.25, 0.5, 1, 3, 6, 9 and 24 hours post dose. For the IV group, urine is collected from each animal at predose and from 0-6 and 6-24 hours post dose. The vehicle used for IV dose groups is a combination of PEG400 with citrate buffer (pH 5.0) or PEG400/Cremphor with DMSO/H2O, and for PO groups was MCT.

Plasma and urine concentrations str quantitated using a LC/MS/MS method. The lower limit of quantitation (LLOQ) of the plasma and urine assays is 0.005 pM. PK parameters are determined by non-compartmental methods using WinNonlin (version 5.2, Pharsight Corporation, Mountain View, CA).

### Example 32. Pharmacodynamics (PD) Study; Enhancement of CD4 and CD8 T Cell Activation in Response to Systemic Anti-CD3 Administration in the Presence of Cbl-b Inhibitor

Female C57BI/6 or Balb/c mice are administered with anti-CD3 antibody (2 ug/mouse, clone 2C1) or an isotype control (2 pg/mouse, hamster IgG) is administered by tail vein injection. A Cbl-b inhibitor is administered PO starting at time 0 (immediately before anti-CD3 administration) and again 8 hrs later. Four hours after anti-CD3 administration, mice are bled and cytokines are quantified in serum via Luminex. Twenty-four hours after anti-CD3 administration, mice are euthanized and activation of CD4 and CD8 T cells quantified in spleens and blood. Expression of 4-1 BB, CD25, CD40L, and CD69 as well as cell surface TCR levels will be quantified by flow cytometry, Serum are collected for cytokine analysis via Luminex.

### Example 33. Tumor PD/Efficacy Study; Evaluation of Tumor Growth and Immune Cell Infiltration in Mice with Syngeneic Tumors Treated Prophylactically or Therapeutically with Cbl-b Inhibitor

Female C57Bl/6 mice age 6-12 weeks are inoculated subcutaneously in the 5th mammary fat pad with 0.1 million E0771 cells in 100 microliters of HBSS+matrigel under manual restraint. For prophylactic studies, a Cbl-b inhibitor is administered PO BIDx3 weeks starting 1 hr prior to tumor inoculation. Three weeks after tumor inoculation, mice are euthanized and tumor, spleen, blood and draining lymph node are harvested and immune cell infiltrate and phenotype are assessed by flow cytometry. Serum are obtained at various time points for cytokine analysis via Luminex. For therapeutic efficacy assessment, tumors are inoculated as described above and allowed to grow until tumors reach a median volume of 120-250 mm³. Dosing with a Cbl-b inhibitor is then be initiated as above and continued until end of study. Tumor volumes and mouse body weight and condition are recorded twice weekly or more as needed until end of study. Efficacy of a Cbl-b inhibitor can also be assessed in additional syngeneic tumor models, including CT26 and TC-1.

### Example 34. Cbl-b and c-cbl SPR Assay

Affinity of binding to Cbl-b and c-Cbl for compounds described herein can be assessed by surface plasmon resonance (SPR) according to the following protocol. All experiments were recorded on a Biacore™ 8K or Biacore™ 8K+(Cytiva) with both surface preparation and experimental measurements performed at 20° C. in an assay buffer consisting of 50 mM HEPES, pH 7.5, 0.15 M NaCl, 0.001% (v/v) Tween® 20, 0.2 mM tris(2-carboxyethyl)phosphine, 0.025% (w/v) carboxymethylated dextran (average MW 10 kDa), 0.2% (w/v) PEG 3350, and 2% DMSO.

Human Cbl-b (residues 40-426) or c-Cbl (residues 47-435) are irreversibly captured to a Series S sensor chip SA (Cytiva 29104992) via an N-terminal avi-tag, biotinylated by co-expression in E. coli with BirA. A surface capture range of 1300-1500 RU of protein is used for both isoforms.

For SPR measurements, 6 concentrations with 2 fold serial dilution are measured with blanks flanking each series for double referencing. Initial concentrations between 20 and 0.5 .M were used depending on the anticipated affinity of the tested compound. SPR sensorgrams are recorded in multi-cycle kinetics format, with a contact time of 60 seconds and a flow rate of 40 pd/min, the dissociation time is varied between 120-1200 seconds aiming for 4-5 half-lives of the measured interaction.

Kinetic and affinity parameters are extracted from the multicycle kinetics data fitting to a 1:1 binding model using the Biacore™ Incyte evaluation software (Cytiva).

### Multi-Point Chaser SPR Assay Variation

For the purposes of this type of experiment the term “chaser compound” refers to a low affinity analogue of the compound under investigation which binds close to saturation at the used concentration and fully dissociates within 120 seconds.

Affinity of binding to Cbl-b and c-Cbl for potent compounds described herein (Kd<10 nM) are assessed by surface plasmon resonance (SPR) using a “Chaser” assay format.

Chaser assay utilize a single cycle kinetics SPR experiment with a contact time of 120 seconds, a flow rate of 50 pd/min and a dissociation time of 450 seconds. Single cycle kinetics titration utilized an initial blank injection and 5 concentrations with 2 fold serial dilution with a maximum concentration of 500 nM, blanked to a preceding 6 point blank single cycle kinetics injection for double referencing.

In the case of potent compounds, the protein-compound half-life cannot be accurately measured using routine fitting of the single cycle titration data. The kd is measured independently by determining the percentage unoccupied compound binding site over time by measuring the binding of a chaser compound measured by SPR.

Chaser binding is measured by a multicycle kinetics SPR experiment using a contact time of 20 seconds, a flow rate of 30 pd/min, and a dissociation time of 120 seconds.

7 injections of a single chaser concentration of 15. M with a preceding blank injection, are recorded spaced out between 674 and 30.263 seconds after the last single cycle kinetics titration injection. The % compound bound at a given time is determined by comparison to a single injection of chaser preceding the single cycle kinetics titration defined below.

% Compound Bound=(1−(RUT/RUT0))*100 where RUT is the observed SPR signal for chaser injection at time T. and R UTO is the observed SPR signal for chaser injected prior to the titration of compound under investigation.

The % compound bound is plotted against time in seconds and fit to a single exponential, where the exponent represents the kd of the compound-protein complex. The single cycle kinetics experiment of the compound is then fit with a fixed kd determined using the chaser.

SPR and the LCK biochemical assay are orthogonal assays: SPR is a protein binding assay while the LCK assay is an enzyme activity assay. SPR measures compound binding affinity to CBL-B/C-CBL, whereas LCK assay measures compound inhibition of CBL-B/C-CBL ubiquitin transfer activity.

### REFERENCES

1: Bachmaier, et. al. Nature (2000) 403:211-216
2. Chiang, et. al. Nature (2000) 403:216-220
3: Ronchi and Haas, Methods Mol Biol. (2012) 832: 197

1. A compound of formula (I): 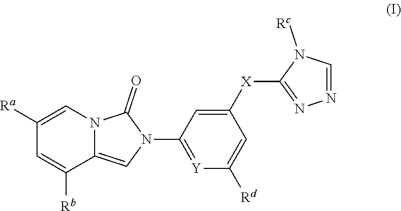
or pharmaceutically acceptable salts thereof,
wherein
X is an optionally substituted C₁-C₃ alkylene chain, wherein one or more methylene units is optionally replaced by optionally substituted 3-6-membered carbocyclyl or optionally substituted 3-6-membered heterocylyl, wherein X is optionally substituted with an optionally substituted group selected from the group consisting of halogen, cyano, and C₁-C₃ aliphatic;
Y is selected from N or C;
R^{a} is selected from the group consisting of —CH₂-A, —CH₂N(H)-A, and —CH₂NH₂;
R^{b} is halogen or optionally substituted C₁-C₃ alkyl;
R^{c} is optionally substituted C₁-C₃ alkyl;
R^{d} is selected from —NR¹R² or hydrogen;
A is optionally substituted C₃-C₇ carbocylyl or optionally substituted 3-6 membered heterocyclyl containing 1-4 heteroatoms each selected from the group consisting of N, O, and S, wherein A is optionally substituted with 1-5 instances of R^{a};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S,
wherein two instances of R^{a1} are optionally taken together to form an optionally substituted C₃-C₇ carbocylyl or optionally substituted 3-6 membered heterocyclyl containing 1-4 heteroatoms each selected from the group consisting of N, O, and S;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S; and
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

2. The compound of claim 1, wherein the compound is of Formula (I-a): 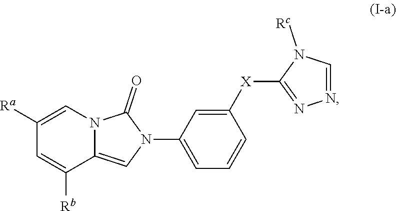
or pharmaceutically acceptable salts thereof.

3. The compound of claim 1, wherein the compound is of Formula (I-b): 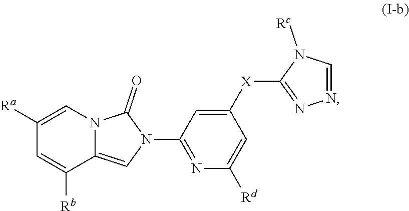
or pharmaceutically acceptable salts thereof.

4. The compound of any of claims 1-3, wherein R^{c} is —CH₃ or —CHF₂.

5. The compound of claim 4, wherein R^{c} is —CHF₂.

6. The compound of claim 4, wherein R^{c} is —CH₃.

7. The compound of any of claims 1-6, wherein X is optionally substituted C₁-C₂ alkylene.

8. The compound of any of claims 1-6, wherein X is optionally substituted C₂ alkylene, wherein one methylene unit is replaced with .

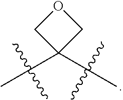 [SMILES: *C1(*)COC1]

9. The compound of any of claims 1-6, wherein X is optionally substituted C₂ alkylene, wherein one methylene unit is replaced with

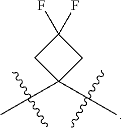 [SMILES: *C1(*)CC(F)(F)C1]

10. The compound of any of claims 1-6, wherein X is optionally substituted C₂ alkylene, wherein one methylene unit is replaced with

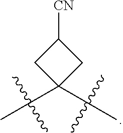 [SMILES: *C1(*)CC(C#N)C1]

11. The compound of any of claims 1-6, wherein X is optionally substituted C₂ alkylene.

12. The compound of any of claims 1-6, wherein X is selected from the group consisting of

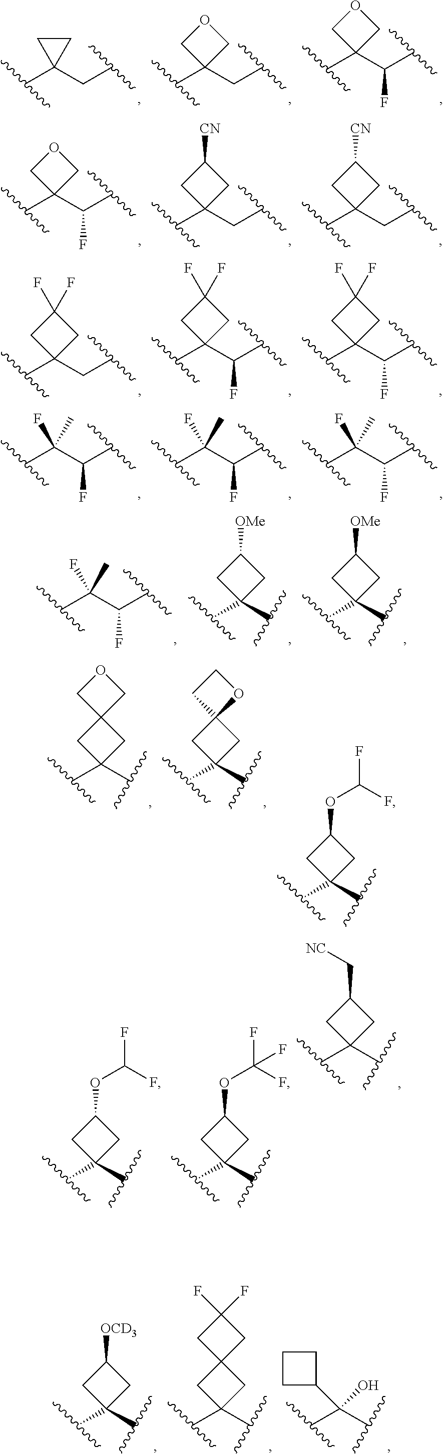

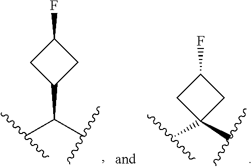

13. The compound of any of claims 1-12, wherein R^{a} is —CH₂-A or —CH₂N(H)-A.

14. The compound of any of claims 1-12, wherein R^{a} is —CH₂NH₂.

15. The compound of claim 13, wherein A is optionally substituted 3-6 membered heterocyclyl containing 1-4 heteroatoms each selected from the group consisting of N, O, and S.

16. The compound of claim 13, wherein A is optionally substituted 3-6 membered carbocylyl.

17. The compound of any of claims 1-13, wherein R^{a} is selected from the group consisting of —CH₂NH₂,

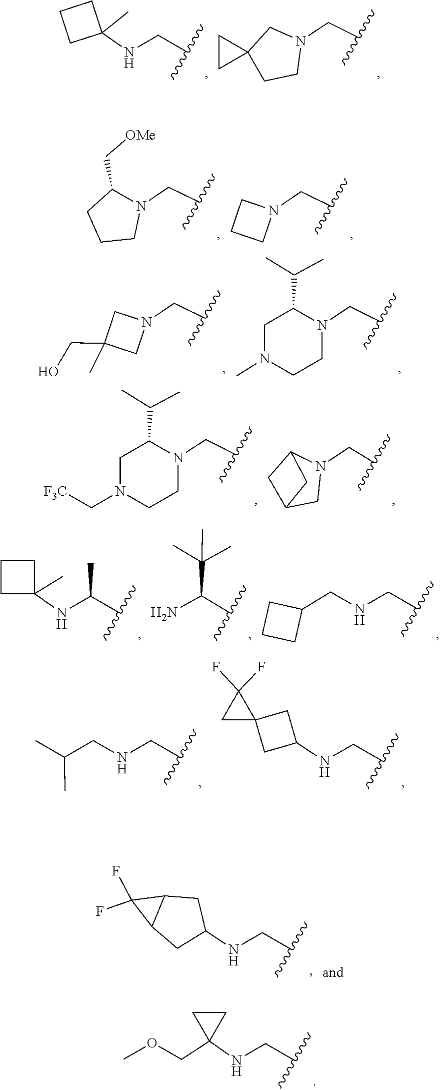

18. The compound of any of claims 1-17, wherein R^{b} is halogen.

19. The compound of claim 18, wherein R^{b} is chloro.

20. The compound of any of claims 1-17, wherein R^{b} is optionally substituted C₁ alkyl.

21. The compound of any of claims 1-17 is R^{b} selected from the group consisting of chloro, —CF₃, and —CHF₂,

22. The compound of any of the claims 1-21, wherein R^{d} is selected from R^{d} is selected from —NR¹R² or hydrogen;

23. The compound of claim 22, wherein R^{d} is hydrogen.

24. The compound of claim 22, wherein R^{d} is

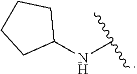 [SMILES: *NC1CCCC1]

25. A compound selected from the group consisting of 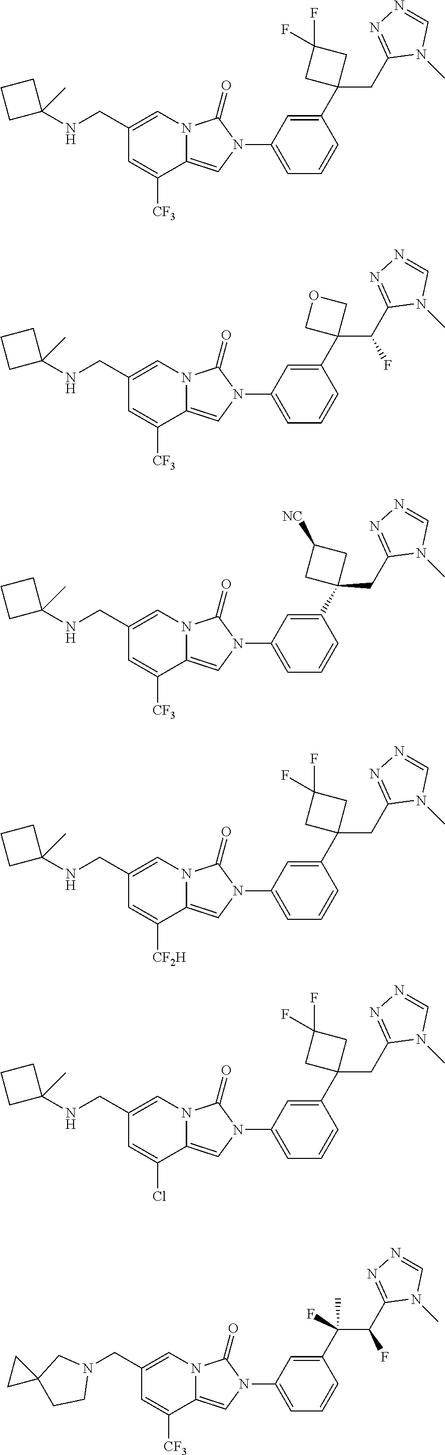 [SMILES: Cn1cnnc1CC1(c2cccc(-n3cc4c(C(F)(F)F)cc(CNC5(C)CCC5)cn4c3=O)c2)CC(F)(F)C1 ; Cn1cnnc1[C@H](F)C1(c2cccc(-n3cc4c(C(F)(F)F)cc(CNC5(C)CCC5)cn4c3=O)c2)COC1 ; Cn1cnnc1C[C@]1(c2cccc(-n3cc4c(C(F)(F)F)cc(CNC5(C)CCC5)cn4c3=O)c2)C[C@H](C#N)C1 ; Cn1cnnc1CC1(c2cccc(-n3cc4c(C(F)F)cc(CNC5(C)CCC5)cn4c3=O)c2)CC(F)(F)C1 ; Cn1cnnc1CC1(c2cccc(-n3cc4c(Cl)cc(CNC5(C)CCC5)cn4c3=O)c2)CC(F)(F)C1 ; Cn1cnnc1[C@@H](F)[C@](C)(F)c1cccc(-n2cc3c(C(F)(F)F)cc(CN4CCC5(CC5)C4)cn3c2=O)c1] 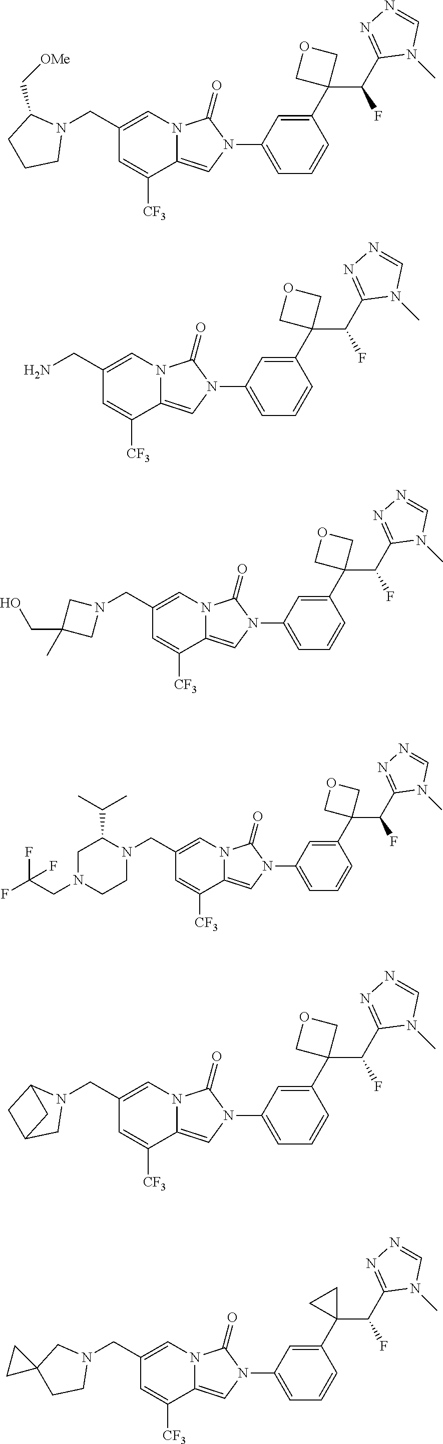 [SMILES: COC[C@H]1CCCN1Cc1cc(C(F)(F)F)c2cn(-c3cccc(C4([C@H](F)c5nncn5C)COC4)c3)c(=O)n2c1 ; Cn1cnnc1[C@H](F)C1(c2cccc(-n3cc4c(C(F)(F)F)cc(CN)cn4c3=O)c2)COC1 ; Cn1cnnc1[C@H](F)C1(c2cccc(-n3cc4c(C(F)(F)F)cc(CN5CC(C)(CO)C5)cn4c3=O)c2)COC1 ; CC(C)[C@H]1CN(CC(F)(F)F)CCN1Cc1cc(C(F)(F)F)c2cn(-c3cccc(C4([C@H](F)c5nncn5C)COC4)c3)c(=O)n2c1 ; Cn1cnnc1[C@H](F)C1(c2cccc(-n3cc4c(C(F)(F)F)cc(CN5CC6CC5C6)cn4c3=O)c2)COC1 ; Cn1cnnc1[C@H](F)C1(c2cccc(-n3cc4c(C(F)(F)F)cc(CN5CCC6(CC6)C5)cn4c3=O)c2)CC1] 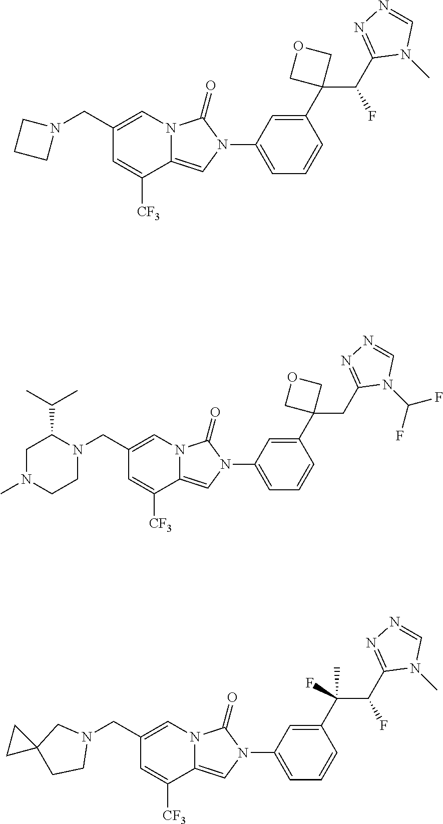 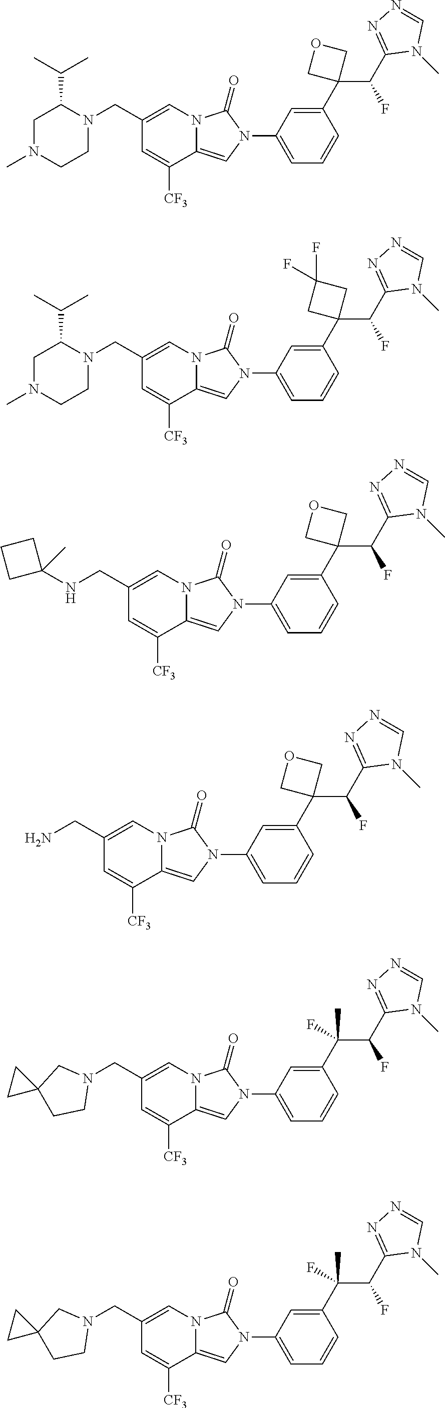 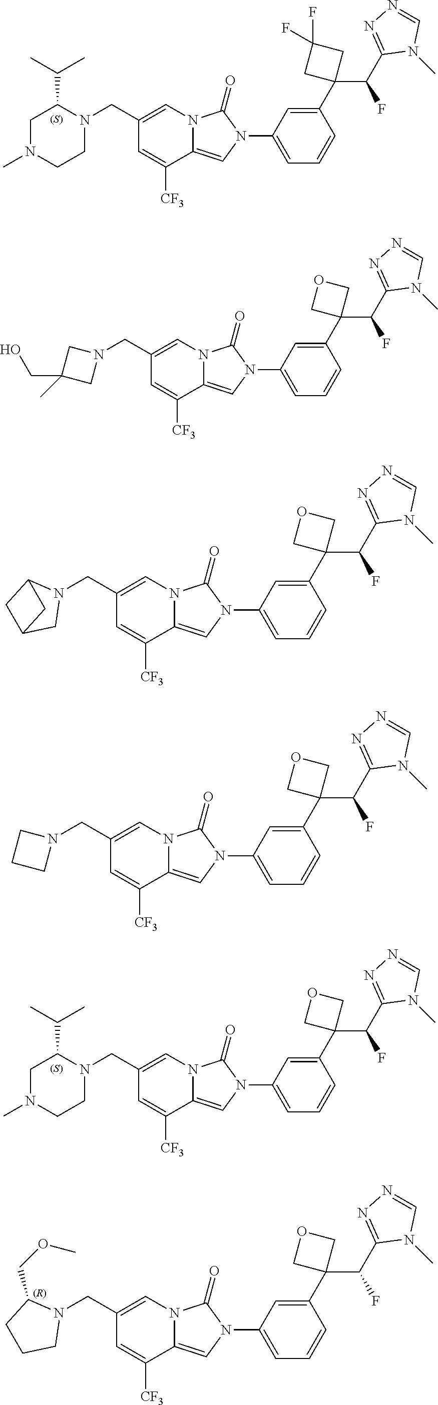 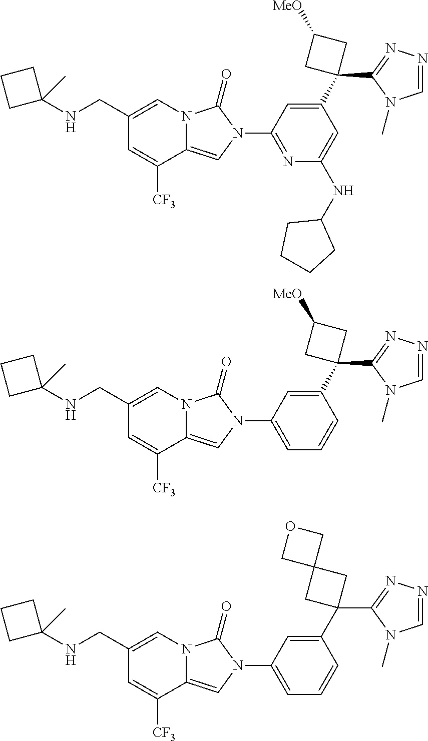 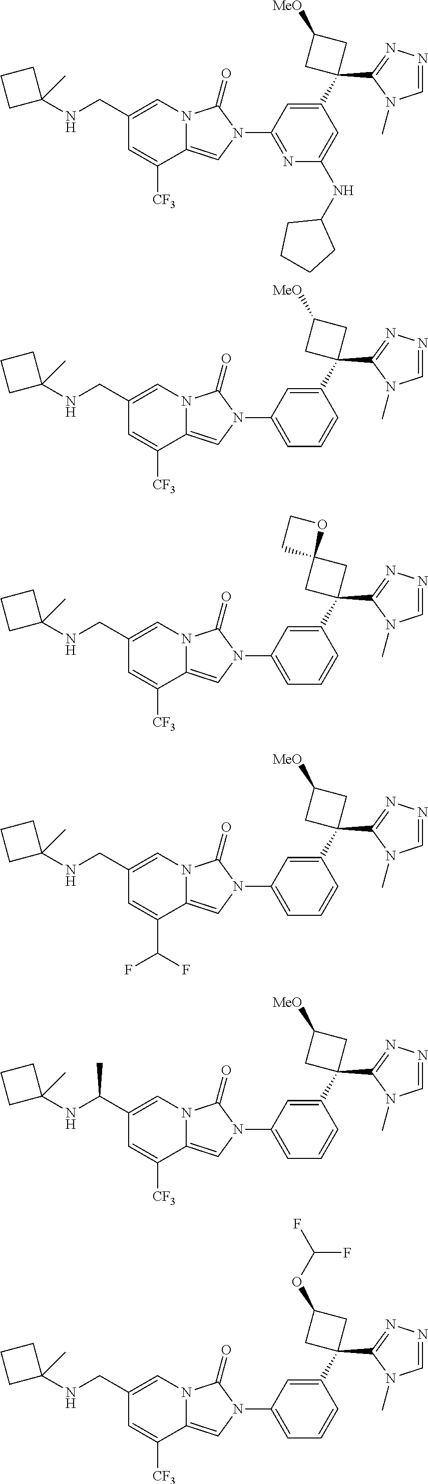 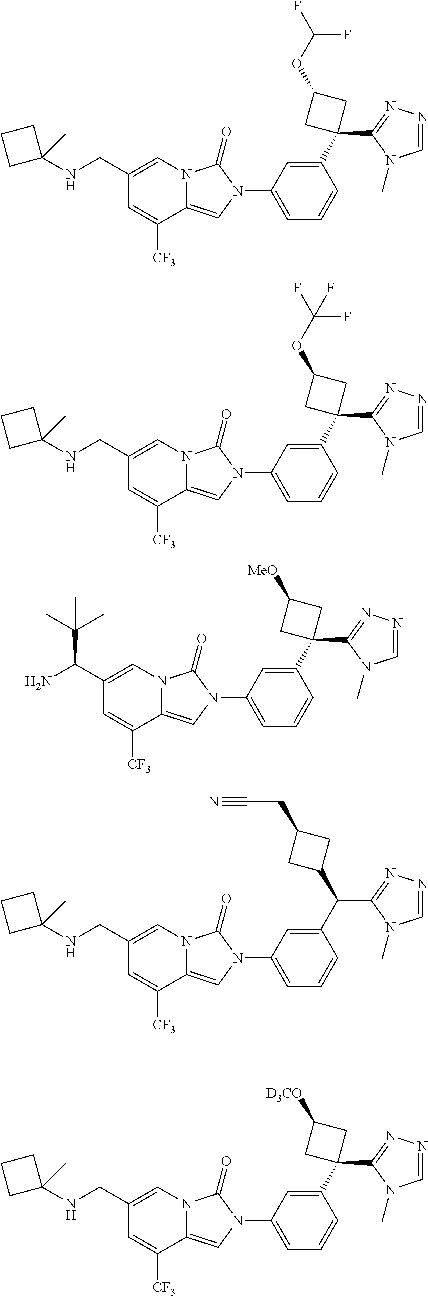 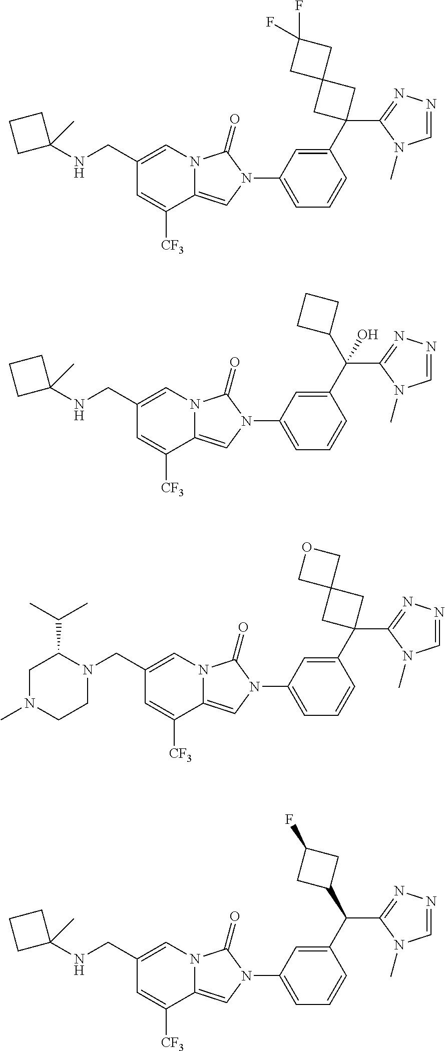 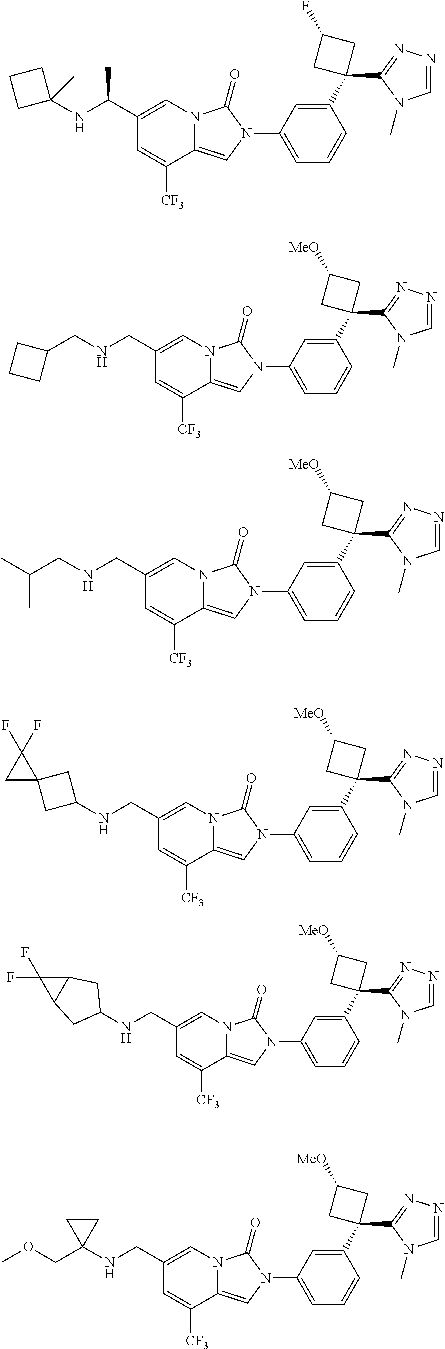 [SMILES: C[C@H](NC1(C)CCC1)c1cc(C(F)(F)F)c2cn(-c3cccc([C@]4(c5nncn5C)C[C@H](F)C4)c3)c(=O)n2c1 ; CO[C@H]1C[C@](c2cccc(-n3cc4c(C(F)(F)F)cc(CNCC5CCC5)cn4c3=O)c2)(c2nncn2C)C1 ; CO[C@H]1C[C@](c2cccc(-n3cc4c(C(F)(F)F)cc(CNCC(C)C)cn4c3=O)c2)(c2nncn2C)C1 ; CO[C@H]1C[C@](c2cccc(-n3cc4c(C(F)(F)F)cc(CNC5CC6(C5)CC6(F)F)cn4c3=O)c2)(c2nncn2C)C1 ; CO[C@H]1C[C@](c2cccc(-n3cc4c(C(F)(F)F)cc(CNC5CC6C(C5)C6(F)F)cn4c3=O)c2)(c2nncn2C)C1 ; COCC1(NCc2cc(C(F)(F)F)c3cn(-c4cccc([C@]5(c6nncn6C)C[C@H](OC)C5)c4)c(=O)n3c2)CC1]
or a pharmaceutically acceptable salt thereof.

26. A pharmaceutical composition comprising a compound of any of claims 1-25 and a pharmaceutically acceptable adjuvant or carrier.

27. A method of treating a disease or condition associated with cell proliferation comprising administering a therapeutically effective amount of a compound of any of claims 1-25 or a pharmaceutical composition of claim 26 to a subject in need thereof.

28. The method of claim 27, wherein the disease or condition associated with cell proliferation is hyperplasia or cancer.

29. The method of claim 28, wherein cancer is a hematologic cancer.

30. The method of claim 29, wherein the hematologic cancer is selected from a group consisting of lymphoma, leukemia, and myeloma.

31. The method of claim 30, wherein cancer is a non-hematologic cancer.

32. The method of claim 31, wherein the non-hematologic cancer is a sarcoma or a carcinoma.

33. The method of any one of claims 27-32, wherein the subject has one or more of increased T-cell activation, increased T-cell proliferation, decreased T-cell exhaustion, decreased T-cell anergy and decreased T-cell tolerance after administration of compound of any of claims 1-15 or a pharmaceutical composition of claim 16.

34. The method of claim 33, wherein increased T-cell activation comprises increased production of a cytokines.

35. The method of claims 27-32, wherein the subject has increased NK-cell activation.

36. The method of 35, the increased NK-cell activation comprises increased production of cytokines.
